(19) United States
(12) Patent Application Publication
(10) Pub. No.: US 2006/0030007 A1
(43) Pub. Date: Feb. 9, 2006

(54) Peptides for metal ion affinity chromatography

(76) Inventors: Devon R.N. Byrd, Fredericksburg, VA (US); Dominic Esposito, Frederick, MD (US); Robert Jason Potter, San Marcos, CA (US); Thomas Chappell, San Marcos, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/857,435

(22) Filed: Jun. 1, 2004

Related U.S. Application Data
(60) Provisional application No. 60/474,220, filed on May 30, 2003.

(51) Int. Cl. C07H 21/04; C12P 21/04; C12N 9/22; C07K 14/195

(52) U.S. Cl. 435/69.7; 435/252.3; 435/320.1; 435/472; 530/350; 536/23.5; 435/199

(57) ABSTRACT

The invention relates generally to affinity peptides having binding activity for metal ion affinity chromatography media. The invention further relates to vectors which encode these affinity peptides and use of these affinity peptides for the purification of biological molecules such as proteins. The invention also relates to fusion proteins comprising affinity peptides of the invention.

### CROSS-REFERENCE TO RELATED APPLICATION

This application claims the benefit of U.S. Provisional Patent Application No. 60/474,220, filed May 30, 2003.

### BACKGROUND OF THE INVENTION

1. Field of the Invention

The invention relates to the biotechnology field. In particular, the invention relates to the fields of protein production and purification. In particular embodiments, the invention relates to affinity peptides that bind metal ion affinity chromatography media.

2. Related Art

Recombinant DNA technology has enabled the production of desired polypeptides in host cells. Such host-produced polypeptides typically are separated from host cell proteins to some degree prior to use. An overview of protein purification techniques is provided in Hopp et al., U.S. Pat. No. 4,782,137.

Affinity chromatography is often the preferred method for protein purification and can often be used to purify proteins from complex mixtures with high yield. Affinity chromatography is based on the ability of proteins to bind non-covalently but specifically to an immobilized ligand for the desired protein; for example, an antibody for a protein antigen. When the specific peptide has affinity to metal ions, isolation of the fusion protein can be done using metal affinity chromatography.

Immobilized Metal Ion Affinity Chromatography (IMAC) is one of the most frequently used techniques for purification of fusion proteins containing affinity sites for metal ions (Porath et al., Nature 258:598-599, 1975). Porath et al. disclose derivatization of a resin with iminodiacetic acid (IDA) and chelating metal ions to the IDA-derivatized resin. The proteins could be immobilized by binding to the metal ion(s) through amino acid residues capable of donating electrons. A number of factors play a role in determining whether a particular protein will bind to the resin, including (1) the conformation of the particular protein, (2) the number of available coordination sites on the immobilized metal ion, (3) the accessibility of protein side chains to the metal ion, and (4) the number of available amino acids for coordination with the immobilized metal ion. Thus, it is often difficult to predict which protein will bind to metal chelate resins and the affinity with which these proteins will bind.

Smith et al. disclose in U.S. Pat. No. 4,569,794 that certain amino acids residues of proteins can bind to the immobilized metal ions, for example, histidine. Smith et al. demonstrate that a fusion protein comprising a desired polypeptide with an attached metal chelating peptide may be purified from contaminants by passing the fusion protein and contaminants through columns containing immobilized metal ions. The metal chelating peptide component of the fusion protein will chelate the immobilized metal ions, while the majority of the contaminants freely pass through the column. By changing the conditions of the column, the fusion protein can be released and then can be collected in relatively pure form.

Even though much has been achieved in metal affinity chromatography, there is still a need for improved compositions and methods for affinity immobilization and purification of proteins.

### SUMMARY OF THE INVENTION

The present invention provides materials and methods for designing and producing peptides having one or more desired characteristics. Examples of desired characteristics include, but are not limited to, reversible binding to affinity matrices (e.g., IMAC matrices), sequences that undergo intein splicing reactions, epitopes, the ability to introduce labels into other polypeptides, and combinations thereof.

In one embodiment, various peptides having a desired characteristic (i.e., ability to bind IMAC matrices) are designed using a novel method based upon the structures of transition metal coordination spheres. The method comprises identifying relevant structural components of proteins that exhibit the desired property and producing peptides comprising the relevant components. In one aspect, methods of the invention may comprise
a) querying protein structures databases for proteins that have the desired property;
b) downloading/acquiring the coordinates of proteins that have the desired property;
c) visualizing the three dimensional structures of proteins that have the desired property;
d) identifying potentially relevant structural components of proteins that exhibit the desired property;
e) producing peptides comprising the potentially relevant components; and
f) testing the synthesized peptides for the desired property. Methods of the invention may further comprise using experimental results to infer rules that relate peptide sequence to desired property. Peptides may be produced using techniques well known in the art, for example, either by constructing nucleic acid molecules encoding the peptides, which may be then used for in vitro and/or in vivo synthesis, or chemically synthesizing the peptides in vitro, for example, using solid phase peptide synthesis.

In one embodiment, the present invention provides peptides that bind to affinity chromatography media (e.g., IMAC media such as iminodiacetic acid, tris(carboxymethyl)ethylene diamine, and nitrilotriacetic resins). In one aspect, the peptide consists essentially of the formula HxHxxHxHxxHxHxx (SEQ ID NO: 1), wherein x is an amino acid, for example one of 20 naturally occurring amino acids. In certain aspects, at least one x residue is lysine, serine or threonine. In other aspects each x independently is lysine, serine, threonine, or tyrosine.

In certain examples of this embodiment, the peptide is HxHxxHxHxxHxHxxHxH (SEQ ID NO: 2). For example, the peptide can be HSHSSHSHSSHSHSSHSH (SEQ ID NO: 3) or HSHKSHYHKKHKHYSHSH (SEQ ID NO: 4). In other examples, the peptide is HSHSSHSHSSHSH (SEQ ID NO: 5), HKHKKHKHKKHKH (SEQ ID NO: 6), HSHSSHYHKKHKH (SEQ ID NO: 7), HYHKKHKHSSHSH (SEQ ID NO: 8), HSHKSHYHSSHKH (SEQ ID NO: 9), or HSHKSHYHKSHSH (SEQ ID NO: 10).

The invention further relates to fusion proteins and other molecules that comprise one or more peptides of the invention. The invention also relates to vectors which encode peptides and/or fusion proteins of the invention. The present invention also provides methods of purifying proteins and/or other biological molecules that comprise one or more peptides of the invention. The invention additionally relates to methods for identifying affinity peptides and preparing affinity peptides having binding activity for metal ion affinity chromatography media.

In one aspect, the present invention is directed to fusion proteins comprising metal chelating affinity peptides, such peptides preferably containing no contiguous histidine residues, and a desired polypeptide or protein attached directly or indirectly to this/these metal chelating affinity peptides, a process for their synthesis by recombinant DNA technology and a process for their purification by IMAC on commonly used IDA resins. When the fusion protein is produced, the desired protein may be isolated and purified by contacting the fusion protein with a matrix containing immobilized metal ions. In one embodiment, the fusion proteins comprising one or more peptides of the invention may be purified from bacterial or non-bacterial sources. The fusion proteins may be expressed in a soluble form and/or secreted from the host as a fusion protein containing a peptide of the invention. A fusion protein according to the present invention may be contacted with an immobilized metal ion containing resin and the fusion protein may be immobilized to allow it to be separated from a mixture.

Therefore, in one aspect of the invention, the present invention provides affinity peptides of the general formula U₁XJYU₂ (SEQ ID NO: 11), wherein, U₁ and U₂ are amino acids independently selected from a group consisting of H, K, or R (histidine, lysine, or arginine), X can be from 1 to 20 amino acid residues, each residue independently drawn from the set of the 20 naturally occurring amino acids commonly found in proteins in either the L or D form of chiral amino acids or a modified amino acid with the proviso that when U₁ is histidine the amino acid of X adjacent to U₁ is not histidine, Y can be from 1 to 20 amino acid residues, each residue independently drawn from the set of the 20 naturally occurring amino acids commonly found in proteins, in either the L or D form of chiral amino acids or Y can be a modified amino acid with the proviso that when U₂ is histidine the amino acid of Y that is adjacent to U₂ is not histidine; and J is drawn from the set: D, E, M, or C (aspartic acid, glutamic acid, methionine, or cysteine). Examples of such peptides are found in Tables 1-6. X and Y may be independently selected, for example, X and Y may be contain the same number of amino acids, which may be the same or different and may be in the same or different order. Alternatively, X and Y may contain a different number of amino acids and/or different amino acids. In some embodiments, X=Y, while in other embodiments, X≠Y. Affinity peptides of this type may be incorporated into fusion proteins, for example, at the N-terminal, C-terminal, and/or at an internal location of the fusion protein. Thus U₁ and/or U₂ may be attached (e.g., via a peptide bond) to a protein sequence of interest.

In yet another aspect of the invention, the present invention provides affinity peptides of the general formula J₁X₁UX₂J₂ (SEQ ID NO: 12), wherein J₁ and J₂ are independently drawn from the set: D, E, or C (aspartic acid, glutamic acid, cysteine); X₁ and X₂ are independently from 1 to 20 amino acid residues drawn from the set of the 20 naturally occurring amino acids commonly found in proteins, either the L or D form of chiral amino acids, and X₁ and/or X₂ can be a modified amino acid; U is drawn from the set: H, K, or R (histidine, lysine, arginine), with the proviso that when U is histidine, the amino acids of X₁ and X₂ that are adjacent to U are not histidine. X₁ and X₂ may be independently selected, for example, X₁ and X₂ may be contain the same number of amino acids, which may be the same or different and may be in the same or different order. Alternatively, X₁ and X₂ may contain a different number of amino acids and/or different amino acids. In some embodiments, X₁=X₂, while in other embodiments, X₁≠X₂. Affinity peptides of this type may be incorporated into fusion proteins, for example, at the N-terminal, C-terminal, and/or at an internal location of the fusion protein. Thus J₁ and/or J₂ may be attached (e.g., via a peptide bond) to a protein sequence of interest.

In another aspect of the invention, the present invention provides affinity peptides of the general formula H(XᵢH)ⱼ (SEQ ID NO: 13) where i=1-6 and j=1-6, with the proviso that when j≧2, at least one pair of Xᵢ adjacent to the same histidine do not have the same number of amino acids. Each Xᵢ may independently be from 1 to 6 amino acid residues drawn from the set of the 20 naturally occurring amino acids commonly found in proteins in either the L or D form of chiral amino acids or a modified amino acid with the proviso that the amino acid of X adjacent to H is not histidine. Affinity peptides of this type may be incorporated into fusion proteins, for example, at the N-terminal, C-terminal, and/or at an internal location of the fusion protein. Thus, the N-terminal histidine and/or the C-terminal histidine may be attached (e.g., via a peptide bond) to a protein sequence of interest.

In yet another aspect of the invention, the present invention provides affinity peptides with the general formula aHbHc (SEQ ID NO: 14), wherein H is histidine; a=zero or more amino acids, drawn from the set of the 20 naturally occurring amino acids commonly found in proteins in either the L or D form of chiral amino acids or a modified amino acid with the proviso that the amino acid of a adjacent to H is not histidine; b=one or more amino acids, drawn from the set of the 20 naturally occurring amino acids commonly found in proteins in either the L or D form of chiral amino acids or a modified amino acid with the proviso that the amino acid of b adjacent to H is not histidine;
and c=zero or more amino acids, drawn from the set of the 20 naturally occurring amino acids commonly found in proteins in either the L or D form of chiral amino acids or a modified amino acid with the proviso that the amino acid of c adjacent to H is not histidine. Affinity peptides of this type may be incorporated into fusion proteins, for example, at the N-terminal, C-terminal, and/or at an internal location of the fusion protein. Thus a and/or c may be attached (e.g., via a peptide bond) to a protein sequence of interest.

In still another aspect of the invention, the present invention provides affinity peptides with the general formula R1-H(XᵢH)ⱼ—R2 (SEQ ID NO: 15) wherein i=an integer from 1 to 10, and j=1-10, with the proviso that when j≧2, at least one pair of Xᵢ adjacent to the same histidine do not have the same number of amino acids. Each Xᵢ may independently be from 1 to 10 amino acid residues drawn from the set of the 20 naturally occurring amino acids commonly found in proteins in either the L or D form of chiral amino acids or a modified amino acid with the proviso that the amino acid of X adjacent to H is not histidine. The amino acid in the position of the R1-proximal “X” may be the same or different as the amino acid in the position of the R2-proximal “X”. The R1-proximal “X” may or may not have the same value for “i” as does the R2-proximal “X”. R1 and R2 may independently be hydrogen, one or more amino acids or a protein sequence of interest. Thus, the N-terminal histidine and/or the C-terminal histidine may be attached (e.g., via a peptide bond) to a protein sequence of interest.

In still another aspect of the invention, the present invention provides affinity peptides with the general formula HzHzzHzH (SEQ ID NO: 16), wherein each z is independently selected from the group consisting of Y and K. Affinity peptides of this type may be incorporated into fusion proteins, for example, at the N-terminal, C-terminal, and/or at an internal location of the fusion protein. Thus the 5′-H and/or the 3′-H may be attached (e.g., via a peptide bond) to a protein sequence of interest.

Typically, affinity peptides of the invention will bind to metal chelate affinity chromatography media when one or more types of metal ions (e.g., Cu²⁺, Ni²⁺, etc.) are bound to these media. Association between the peptide and the metal ion is preferably reversible. Once a fusion protein comprising an affinity peptide of the invention has been allowed to associate or adsorb with the immobilized metal ion, any undesired components present in the solution comprising the fusion protein may be washed off, and the fusion protein can be disassociated or eluted from the metal ion/adsorbent. Suitable conditions for washing and elution are known to those skilled in the art and other conditions may be developed using routine experimentation. Suitable examples of elution conditions include, but are not limited to, addition of one or more molecules that compete with the fusion protein for binding to the immobilized metal ion (e.g., imidazole) and altering the pH of the solution (e.g., increasing or decreasing).

The present invention also provides fusion proteins which comprise one or more (e.g., one, two, three, four, five, six, seven, etc.) affinity peptides of the invention, as well as methods for preparing, isolating and/or purifying these fusion proteins. Fusion proteins of the invention, as well as fusion proteins prepared by methods of the invention, may contain one or more affinity peptides located at or near the carboxyl terminus, at or near the amino terminus, and/or internally. For example, the invention includes, in part, multi-component fusion proteins, as well as methods for preparing such fusion proteins, where a single affinity peptide is located internally between functional domains of a desired protein. In particular embodiments, internal affinity peptides may be introduced into a desired protein to disrupt one or more functional domains and thus alter one or more activities of the desired protein.

In another embodiment, the present invention provides a method for identifying a peptide that binds to an immobilized metal ion, such as an immobilized metal ion associated with a chromatography matrix, by identifying a segment in a polypeptide that includes at least 4 histidine residues that make up at least 25% of the segment.

The invention provides methods for isolating and/or purifying molecules which comprise affinity peptides of the invention. Examples of such molecules include carbohydrates (e.g., monosaccharides, disaccharides, trisaccharides, polysaccharides, etc.), nucleic acids (e.g., DNA, RNA, DNA-RNA hybrids), lipids, fatty acids, and proteins.

Typically, methods for purifying affinity peptides of the invention and fusions proteins which comprise these peptides will involve contacting the affinity peptides or fusion proteins with one or more metal chelate affinity chromatography medium or resin under conditions where said fusion protein of peptide binds to said resin to produce a resin-fusion protein complex; washing said resin-fusion protein complex with a buffer to remove unbound material, and eluting said bound fusion protein from the washed resin-fusion protein complex wherein said eluted fusion protein is purified. However, in specific embodiments, one or more metal ions may be bound to the affinity peptides before the affinity peptides or fusion proteins which comprise the affinity peptides are contacted with metal chelate affinity chromatography media.

In many embodiments of the invention, molecules which are isolated and/or purified by methods of the invention will be recovered in substantially pure form and/or will retain one or more biological activities.

The present invention also relates to compositions for carrying out methods of the invention and to compositions made while carrying out methods of the invention. Such compositions may comprise any one or a combination of the elements used in methods of the invention (e.g. one or more fusion proteins, one or more affinity chromatography resins, etc.) and/or they may also comprise one or more nucleic acid molecules encoding an affinity peptide of the invention and/or a fusion protein comprising one or more affinity peptide of the invention and a host cell. Preferably, the compositions of the invention comprise at least one component selected from the group consisting of one or more affinity peptides, one or more fusion proteins comprising such affinity peptides, and one or more nucleic acid molecules encoding such affinity peptides and/or fusion proteins.

The present invention also encompasses kits for practicing the methods of the invention (e.g., methods of making proteins, methods of purifying proteins, etc). A kit for producing a protein according to the methods of the invention may comprise one or more containers. Such containers may contain a variety of components, for example, one or more nucleic acid molecules encoding an affinity peptide, one or more recombination proteins, one or more restriction enzymes, one or more topoisomerase enzymes, one or more affinity chromatography resins, one or more host cells, one or more cell extracts for in vitro transcription and/or translation. In certain such embodiments, kits may comprise one or more containers containing one or more nucleic acid molecules encoding affinity peptides, one or more containers containing one or more recombination enzymes, and one or more containers containing one or metal ion affinity chromatography medium. Kits of the invention may comprise one or more additional components such as one or more containers containing one or more components selected from the group consisting of one or more polymerases, one or more buffers, one or more primers, one or more vectors, one or more nucleic acid molecules comprising one or more promoter sequences, and one or more nucleotides.

Other embodiments of the invention will be apparent to one or ordinary skill in the art in light of what is known in the art, in light of the following drawings and description of the invention, and in light of the claims.

### BRIEF DESCRIPTION OF THE FIGURES

FIG. 1 is a schematic representation of cis and trans intein reactions.

FIG. 2 is a schematic representation of simultaneous removal of an affinity peptide and a peptide having an intein site from fusion proteins of the invention.

FIG. 3 is a schematic representation of removal of an affinity peptide of the invention by intein trans-splicing.

FIG. 4 is a schematic representation of addition of an affinity peptide of the invention to a protein of interest via intein trans-splicing.

FIG. 5 is a schematic representation of replacement of an affinity peptide of the invention containing an intein sequence with an epitome tag via intein trans-splicing.

FIG. 6 is a composite of phosphorimaging data showing the binding characteristics of the indicated peptides analyzed as described in Example 3.

FIG. 7 is a is a composite of phosphorimaging data showing the binding characteristics of the indicated peptides analyzed as described in Example 3.

FIG. 8 shows the results of an SDS-PAGE analysis of the indicated peptides as described in Example 4.

### DETAILED DESCRIPTION OF THE INVENTION

Definitions.

In order to provide a clear and consistent understanding of the specification and claims, including the scope to be given such terms, the following definitions are provided.

As used herein, the following is the set of 20 naturally occurring amino acids commonly found in proteins and the one and three letter codes associated with each amino acid

| Full name | Three-letter Code | One-letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, “non-natural amino acid” is any analog of a natural amino acid that may be incorporated into a peptide and/or fusion protein of the invention. Examples of non-natural amino acids include, but are not limited to, non-natural amino acids such as 2-methylvaline, 2-methylalanine, (2-i-propyl)-β-alanine, phenylglycine, 4-methylphenylglycine, 4-isopropylphenylglycine, 3-bromophenylglycine, 4-bromophenylglycine, 4-chlorophenylglycine, 4-methoxyphenylglycine, 4-ethoxyphenylglycine, 4-hydroxyphenylglycine, 3-hydroxyphenylglycine, 3,4-dihydroxyphenylglycine, 3,5-dihydroxyphenylglycine, 2,5-dihydrophenylglycine, 2-fluorophenylglycine, 3-fluorophenylglycine, 4-fluorophenylglycine, 2,3-difluorophenylglycine, 2,4-difluorophenylglycine, 2,5-difluorophenylglycine, 2,6-difluorophenylglycine, 3,4-difluorophenylglycine, 3,5-difluorophenylglycine, 2-(trifluoromethyl)phenylglycine, 3-(trifluoromethyl)phenylglycine, 4-(trifluoromethyl)phenylglycine, 2-(2-thienyl)glycine, 2-(3-thienyl)glycine, 2-(2-furyl)glycine, 3-pyridylglycine, 4-fluorophenylalanine, 4-chlorophenylalanine, 2-bromophenylalanine, 3-bromophenylalanine, 4-bromophenylalanine, 2-naphthylalanine, 3-(2-quinoyl)alanine, 3-(9-anthracenyl)alanine, 2-amino-3-phenylbutanoic acid, 3-chlorophenylalanine, 3-(2-thienyl)alanine, 3-(3-thienyl)alanine, 3-phenylserine, 3-(2-pyridyl)serine, 3-(3-pyridyl)serine, 3-(4-pyridyl)serine, 3-(2-thienyl)serine, 3-(2-furyl)serine, 3-(2-thiazolyl)alanine, 3-(4-thiazolyl)alanine, 3-(1,2,4-triazol-1-yl)-alanine, 3-(1,2,4-triazol-3-yl)-alanine, hexafluorovaline, 4,4,4-trifluorovaline, 3-fluorovaline, 5,5,5-trifluoroleucine, 2-amino-4,4,4-trifluorobutyric acid, 3-chloroalanine, 3-fluoroalanine, 2-amino-3-fluorobutyric acid, 3-fluoronorleucine, 4,4,4-trifluorothreonine, L-allylglycine, tert-Leucine, propargylglycine, vinylglycine, S-methylcysteine, cyclopentylglycine, cyclohexylglycine, 3-hydroxynorvaline, 4-azaleucine, 3-hydroxyleucine, 2-amino-3-hydroxy-3-methylbutanoic acid, 4-thiaisoleucine, acivicin, ibotenic acid, quisqalic acid, 2-indanylglycine, 2-aminoisobutyric acid, 2-cyclobutyl-2-phenylglycine, 2-isopropyl-2-phenylglycine, 2-methylvaline, 2,2-diphenylglycine, 1-amino-1-cyclopropanecarboxylic acid, 1-amino-1-cyclopentanecarboxylic acid, 1-amino-1-cyclohexanecarboxylic acid, 3-amino-4,4,4-trifluorobutyric acid, 3-phenylisoserine, 3-amino-2-hydroxy-5-methylhexanoic acid, 3-amino-2-hydroxy-4-phenylbutyric acid, 3-amino-3-(4-bromophenyl)propionic acid, 3-amino-3-(4-chlorophenyl)propionic acid, 3-amino-3-(4-methoxyphenyl)propionic acid, 3-amino-3-(4-fluorophenyl)propionic acid, 3-amino-3-(2-fluorophenyl)propionic acid, 3-amino-3-(4-nitrophenyl)propionic acid, and 3-amino-3-(1-naphthyl)propionic acid. These non-natural amino acids are commercial available from the following commercial suppliers including Aldrich, Sigma, Fluka, Lancaster, ICN, TCI, Advanced ChemTech, Oakwood Products, Indofine Chemical Company, NSC Technology, PCR Research Chemicals, Bachem, Acros Organics, Celgene, Bionet Research, Tyger Scientific, Tocris, Research Plus, Ash Stevens, Kanto, Chiroscience, and Peninsula Lab. The following amino acids can be synthesized according to literature procedures: 3,3,3-trifluoroalanine (Sakai, T.; et al. Tetrahedron 1996, 52, 233) and 3,3-difluoroalanine (D'Orchymont, H. Synthesis 1993, 10, 961).

As used herein, the term “peptide” refers to a molecule which is formed by the contiguous linkage of amino acids connected via peptide bonds. As one skilled in the art would recognize, the term peptide includes molecules that contain components other than amino acids (e.g., peptide-nucleic acids, fatty acid molecules, sugar molecules, etc.) or amino acids connected via covalent bonds other than peptide bonds (e.g., disulfide bonds, ester bonds, glycosidic bonds, etc.). Typically, peptides may comprise from 2 to about 50 amino acid residues. Polypeptides typically include at least 51 amino acid resides.

As used herein, the term “protein,” refers to a molecule which is formed by the contiguous linkage of amino acids via peptide bonds. As noted above for peptides, the term protein includes molecules that contain components other than amino acids (e.g., peptide-nucleic acids, fatty acid molecules, sugar molecules, etc) or amino acids connected via covalent bonds other than peptide bonds (e.g., disulfide bonds, ester bonds, glycosidic bonds, etc.). Typically, a protein may comprise about 25 amino acid residues or more.

As used herein, the term “metal affinity peptide” means a peptide which binds to one or more metal ions such as Cu⁺², Co⁺², or Ni⁺², Zn⁺², Ac⁺³, and Fe⁺³. Typically, affinity peptides will have sufficient affinity for the metal ions so that protein connected (e.g., covalently or non-covalently) to the affinity peptides can be purified using methods of the invention.

As used herein, the term “fusion protein” means a protein that comprises at least two stretches of contiguous amino acids that are not naturally found in the same protein. Included within the scope of the term fusion protein is the term “fusion peptides.” Thus, fusion proteins of the invention which comprise, for example, a five amino acid affinity peptide connected to a ten amino acid peptide are included within the scope of the invention.

As used herein, the term “promoter” shall mean a type of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions. Examples of promoters suitable for use in the present invention include, but are not limited to, an SP6 promoter, a CMV promoter, an SV40 promoter, a bacteriophage promoter, a bacteriophage T7 gene 10 promoter, a host cell native promoter.

As used herein, the phrases “proteolytic site” or “protease site” shall refer to any amino acid sequence recognized by any proteolytic enzyme. In the present case, a fusion protein of the present invention may contain such a proteolytic site between the protein of interest and the affinity peptide and/or other amino acid sequences so that the protein of interest may be separated easily from these heterologous amino acid sequences.

As used herein, the phrase “recombination site” shall mean any nucleic acid that can serve as a substrate in a site-specific recombination reaction. Such recombination sites may be wild-type or naturally occurring recombination sites, or modified, variant, derivative, or mutant recombination sites. Examples of recombination sites for use in the invention include, but are not limited to, phage-lambda recombination sites (such as attP, attB, attL, and attR and mutants or derivatives thereof) and recombination sites from other bacteriophages such as phi80, P22, P2, 186, P4 and P1 (including lox sites such as loxP and loxP511).

Preferred recombination proteins and mutant, modified, variant, or derivative recombination sites for use in the invention include those described in U.S. Pat. Nos. 5,888,732, 6,143,557, 6,171,861, 6,270,969, and 6,277,608 and in U.S. application Ser. No. 09/438,358 (filed Nov. 12, 1999), based upon U.S. provisional application No. 60/108,324 (filed Nov. 13, 1998). Mutated att sites (e.g., attB 1-10, attP 1-10, attR 1-10 and attL 1-10) are described in U.S. application Ser. No. 09/517,466, filed Mar. 2, 2000, and Ser. No. 09/732,914, filed Dec. 11, 2000 (published as 2002 0007051-A1) the disclosures of which are specifically incorporated herein by reference in their entirety. Other suitable recombination sites and proteins are those associated with the GATEWAY™ Cloning Technology available from Invitrogen Corporation, Carlsbad, Calif., and described in the product literature of the GATEWAY™ Cloning Technology, the entire disclosures of all of which are specifically incorporated herein by reference in their entireties.

As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, ΦC31, Cin, Tn3 resolvase, TndX, XerC, XerD, TnpX, Hjc, SpCCE1, and ParA.

As used herein, the term “topoisomerase recognition site” or “topoisomerase site” means a defined nucleotide sequence that is recognized and bound by a site specific topoisomerase. For example, the nucleotide sequence 5′-(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; and PCT/US98/12372 also incorporated herein by reference). In comparison, the nucleotide sequence 5′-GCAACTT-3′ is the topoisomerase recognition site for type IA E. coli topoisomerase III.

As used herein, the phrase “recombinational cloning” refers to a method, such as that described in U.S. Pat. Nos. 5,888,732; 6,143,557; 6,171,861; 6,270,969; and 6,277,608 (the contents of which are fully incorporated herein by reference), whereby segments of nucleic acid molecules or populations of such molecules are exchanged, inserted, replaced, substituted or modified, in vitro or in vivo. Preferably, such cloning method is an in vitro method.

Cloning systems that utilize recombination at defined recombination sites have been previously described in U.S. Pat. No. 5,888,732, U.S. Pat. No. 6,143,557, U.S. Pat. No. 6,171,861, U.S. Pat. No. 6,270,969, and U.S. Pat. No. 6,277,608, and in pending U.S. application Ser. No. 09/517,466 filed Mar. 2, 2000, and in published United States application no. 2002 0007051-A1, all assigned to the Invitrogen Corporation, Carlsbad, Calif., the disclosures of which are specifically incorporated herein in their entirety. In brief, the GATEWAY™ Cloning System described in these patents and applications utilizes vectors that contain at least one recombination site to clone desired nucleic acid molecules in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

As used herein “substantially pure” means that the desired purified protein is essentially free from contaminating cellular contaminants which are associated with the desired protein in nature.

As used herein, the term “vector” refers to a nucleic acid molecule (preferably DNA) that provides a useful biological or biochemical property to an insert. A vector may be a nucleic acid molecule comprising all or a portion of a viral genome. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more recognition sites (e.g., two, three, four, five, seven, ten, etc. recombination sites, restriction sites, and/or topoisomerases sites) at which the sequences can be manipulated in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Other terms used in the fields of recombinant nucleic acid technology and molecular and cell biology as used herein will be generally understood by one of ordinary skill in the applicable arts.

Overview

The present invention provides methods of identifying peptides having one or more desired characteristics and peptides comprising such characteristics. The present invention also relates to fusion proteins comprising peptides of the invention fused to proteins of interest. Such fusions may be made at the N-terminal, C-terminal and/or one or more internal location in the sequence of a protein of interest. The present invention also encompasses nucleic acid molecules encoding peptides of the invention as well as nucleic acid molecules encoding fusion proteins comprising peptides of the invention. The present invention also comprises host cells transfected with such nucleic acid molecules.

Designing Peptides Having Desired Characteristics

In one aspect, the present invention provides methods of designing peptides and/or proteins having one or more desired characteristics. Methods may involve querying protein structures databases for proteins that have a desired property, which may be related to the desired characteristic. For example, a suitable database (e.g., the NCBI Molecular Modeling Database (MMDB)) can be searched to identify proteins having one or more desired property (e.g., affinity for a particular ligand or class of ligand, a desired enzymatic activity, etc.). As set forth in more detail below, to design peptides having the characteristic of binding metal ions, proteins having the property of binding metal ions or of incorporating metal ions into their structure were identified.

Once a set of proteins having the desired property are identified, the three dimensional structures of each of the proteins may be obtained. Typically, this may be accomplished by downloading the coordinates of the proteins from the database. The three dimensional structure of protein may then be visualized. Typically, the portion of the protein associated with the desired property (e.g., binding site, catalytic site, etc.) is displayed and potentially relevant structural components of proteins that exhibit the desired property are identified. Structural components are identified as relevant if they appear in multiple proteins having the desired properties. For example, a structural component may be identified as relevant if it appears in from about 10% to about 100%, from about 15% to about 100%, from about 20% to about 100%, from about 25% to about 100%, from about 35% to about 100%, from about 50% to about 100%, from about 75% to about 100%, from about 15% to about 75%, from about 20% to about 75%, from about 25% to about 75%, from about 35% to about 75%, from about 50% to about 75%, from about 15% to about 50%, from about 20% to about 50%, from about 25% to about 50%, or from about 35% to about 50% of the proteins having the property.

Relevant structural components may be those portions of the protein that interact with one or more ligand (e.g., a substrate, a cofactor, etc.). For proteins that bind one or more metal ions, a relevant structural feature may be an amino acid residue that coordinates with the metal ion. Moreover, relevant structural components may be those portions of the protein that provide the appropriate structural geometry to facilitate the interaction of the same or other portions of the protein with one or more ligands.

Once relevant structural features are identified, peptides may be produced that incorporate one or more relevant structural features. Such peptides may be produced using techniques well known in the art. For example, nucleic acid molecules encoding the peptides may be prepared and introduced into host cells and the peptide expressed from the nucleic acid molecules. Alternatively, nucleic acid molecules encoding peptides may be used as templates in an in vitro transcription/translation process and the peptides may be produced in vitro (see, for example, WO 02/072890 and published U.S. patent application 2002-0168706 A1). Peptides may also be synthesized using standard solid phase synthesis techniques (see, for example, M. Bodanzsky, “Principles of Peptide Synthesis,” 1st and 2nd revised ed., Springer-Verlag, New York, N.Y., 1984 and 1993; Stewart and Young, “Solid Phase Peptide Synthesis,” 2nd ed., Pierce Chemical Co., Rockford, Ill., 1984; Fox J E. Multiple peptide synthesis. Mol Biotechnol. 3:249-258, 1995; Kiso Y, Fujii N, Yajima H. New disulfide bond-forming reactions for peptide and protein synthesis. Braz J Med Biol Res. 27:2733-2744, 1994; Bongers J, Heimer E P. Recent applications of enzymatic peptide synthesis. Peptides. 15:183-193, 1994; Wade J D, Tregear G W. Solid phase peptide synthesis: recent advances and applications. Australas Biotechnol. 3:332-336, 1993; Fields G B, Noble R L. Solid phase peptide synthesis utilizing 9-fluorenylmethoxycarbonyl amino acids. Int J Pept Protein Res. 35:161-214, 1990; Newton R, Fox J E. Automation of peptide synthesis. Adv Biotechnol Processes. 10:1-24, 1988; Barany G, Kneib-Cordonier N, Mullen D G. Solid-phase peptide synthesis: a silver anniversary report. Int J Pept Protein Res. 30:705-739, 1987; Bodanszky M. In search of new methods in peptide synthesis. A review of the last three decades. Int J Pept Protein Res. 25:449-474, 1985; Chaiken I M. Semisynthetic peptides and proteins. CRC Crit Rev Biochem. 11:255-301, 1981; Fridkin M, Patchornik A. Peptide synthesis. Annu Rev Biochem. 43:419-443, 1974; Merrifield R B. Solid-phase peptide synthesis. Adv Enzymol Relat Areas Mol. Biol. 32:221-296, 1969; and U.S. Pat. No. 4,748,002 (Semi-automatic, solid-phase peptide multi-synthesizer and process for the production of synthetic peptides by the use of the multi-synthesizer) to Neimark et al.).

Once peptides have been produced, they may be tested for desired characteristics. For example, if peptides having metal ion affinity are desired, the peptides having putative characteristics relevant for metal ion binding may be assayed for metal binding activity. For example, peptides may be produced and then may be used to contact an immobilized-metal-ion-containing chromatography matrix. Such matrices are well known in the art (see, for example, U.S. Pat. Nos. 4,569,794, 4,877,830, 5,932,102, 6,365,147 and 6,479,300 see also WO 03/000708). Optionally, peptides to be tested may incorporate one or more detectable moieties (e.g., fluorophores, chromophores, radiolabels, enzymes etc). Peptides may be contacted with a matrix and the amount of the peptide that binds to the matrix may be determined, for example, by quantifying the detectable moiety. In addition to detecting binding of the peptide to the matrix, suitable condition for elution from the matrix may also be determined, for example, by contacting matrix-bound peptide with a solution designed to elute the peptide from the matrix and testing the solution for the presence of eluted peptide.

Results of binding assays may be used to further refine the structures of peptides of the invention. For example, experimental results may be used to infer rules that relate peptide sequence to one or more desired characteristic and additional peptides may be produced to incorporate relevant structural features identified.

A suitable program for analyzing the three dimensional structure of proteins having one or more desired properties is the SwissPDBViewer. This program generates a three dimensionally rotatable, translatable, and magnifiable representation of a protein, as well as other atoms present in the crystal from which the coordinates were derived. Using functions of the software, the portion of the protein associated with the desired property (e.g., binding site, catalytic site, etc.) may be located within the virtual three dimensional space defined by the protein. For example, when the desired property is metal binding, portions of the protein binding one or more metal ions may be located. The image of the protein may be modified to display only amino acid residues present in a coordination sphere (i.e., amino acids located sufficiently close to a location of interest). A suitable coordination sphere is an approximately 4-6 Å sphere around the portion of the protein having the desired property (e.g., metal ion binding site) using another function of the program. The spatial orientation and relationship of such residues may be identified, and one or more images captured. This process may be repeated for a sufficient number of proteins so that testable predictions may be made about the structure of a peptide having a desired characteristic (e.g., capable of coordinating a particular metal atom).

Peptides of the Invention.

Methods of the invention may be used to design peptides having one or more desired characteristic. One example of a desired characteristic is binding of the peptides of the invention to immobilized metal ions. Such peptides have a wide variety of uses, for example, they may be used in purification of recombinant polypeptides and/or proteins comprising the peptides.

Using the method described above, a number of relevant structural features for binding of metal ions were identified regarding the structure of protein-nickel coordination spheres. His (H), Cys (C), Met (M), Asp (D), Glu (E), Gln (O), Tyr (Y), Gly (G) residues were present in at least one structure and did not exhibit any positional bias, relative to the primary structure of the protein. Histidines, when more than one was present in a coordination sphere, were never adjacent in the primary structure of the protein. In all cases, they were interspersed by one to many residues. Thus, adjacency of histidines is not a requirement for nickel coordination. Acidic amino acids (D and/or E) were almost always present in a coordination sphere. Sulfur-containing residues (M and/or C) were often present in a coordination sphere. Acidic and sulfur-containing residues rarely occur together in a coordination sphere.

Based upon these observations, peptide sequences embodying one or more of the above properties were inferred from the structural data. Because there was no apparent positional bias, the predicted peptide sequences were permuted to encompass possible structural variations. Thus, a peptide of the invention may comprise one or more amino acids drawn from the group: Gly, Ala (A), Val (V), Leu (L), Ile (1), Pro (P), Phe (F), Y, Trp (W), Ser (S), Thr (T), Asn (N), Gln (O), Cys (C), M, D, E, H, Lys (K), Arg (R). In a preferred embodiment, a peptide of the invention may comprise one or more amino acids drawn from the group: H, C, M, D, E, Q, Y, or G. In particular, peptides of the invention will contain no adjacent histidines.

In another aspect of this embodiment, provided herein is a peptide that consists essentially of the formula HxHxxHxHxxHxHxx (SEQ ID NO: 1), wherein x is an amino acid, for example one of 20 naturally occurring amino acids. In certain aspects, the peptide has the formula HxHxxHxHxxHxHxx (SEQ ID NO: 1). In certain aspects, at least one, two, three, four, five, six, seven, eight, or nine x residues is tyrosine, lysine, serine or threonine. In other aspects each x independently is serine, threonine, lysine or tyrosine. “Consists essentially of” with respect to peptides of the invention means that the peptide can include up to 10 additional amino acids provided that the amino acids do not completely suppress the ability of the peptide, or a fusion protein that includes the peptide, to bind immobilized metal ions. In certain aspects, the peptides include 10, 9, 8, 7, 6, 5, 4, 3, 2, or 1 additional amino acid. In certain aspects the additional amino acids include at least one histidine residue. In certain aspects the additional amino acids are HxH, where x is any amino acid. For example, x in the HxH sequence can be S, T, K, or Y, in certain aspects the HxH sequence is HSH.

In certain examples of this embodiment, the peptide is HxHxxHxHxxHxHxxHxH (SEQ ID NO: 2). For example, the peptide can be HSHSSHSHSSHSHSSHSH (SEQ ID NO: 3) or HSHKSHYHKKHKHYSHSH (SEQ ID NO: 4). In other examples, the peptide is HSHSSHSHSSHSH (SEQ ID NO: 5), HKHKKHKHKKHKH (SEQ ID NO: 6), HSHSSHYHKKHKH (SEQ ID NO: 7), HYHKKHKHSSHSH (SEQ ID NO: 8), HSHKSHYHSSHKH (SEQ ID NO: 9), or HSHKSHYHKSHSH (SEQ ID NO: 10). Other embodiments of the invention include 2, 3, 4, 5, 6, 7, 8, 9, or 10 tandem repeats of peptides provided herein.

Other peptides of the present invention include HKHKKHKHKKHK (SEQ ID NO: 17), HKHKKHYH (SEQ ID NO: 18), H K H K Y H Y H (SEQ ID NO: 19), H K H Y K H K H (SEQ ID NO: 20), H K H Y K H Y H (SEQ ID NO: 21), H K H Y Y H K H (SEQ ID NO: 22), H K H Y Y H Y H (SEQ ID NO: 23), HSHKSHYHKSHSH (SEQ ID NO: 10), HSHSSHYHKKHKH (SEQ ID NO: 7), H Y H K K H K H (SEQ ID NO: 24), HYHKKHKHSSHSH (SEQ ID NO: 8), H Y H K K H Y H (SEQ ID NO: 25), H Y H K Y H K H (SEQ ID NO: 26), H Y H K Y H Y H (SEQ ID NO: 27), H Y H Y K H K H (SEQ ID NO: 28), H Y H Y K H Y H (SEQ ID NO: 29), H Y H Y Y H K H (SEQ ID NO: 30). Other examples of histidine-rich peptides include HKHKHKHKHKH (SEQ ID NO: 31), HSHSHSHSHSHG (SEQ ID NO: 32), and HSHSHSHSHSHS (SEQ ID NO: 33).

The invention further relates to fusion proteins comprising (1) a protein, or fragment thereof, and (2) a peptide of the invention. For example, the fusion protein can include 1, 2, 3, 4, 5, 6, 7, 8, 9, 10 copies of the peptides provided herein, for example arranged consecutively on a fusion protein.

In particular embodiments, the invention includes a fusion protein having a desired activity (e.g., enzymatic activity, binding activity, etc.) and comprising a peptide of the invention. Desired activities may be any activity known to those skilled in the art. In some embodiments, a fusion protein of the invention may comprise one or more enzymatic activities including, but not limited to, polymerase activity (e.g., DNA-dependent DNA polymerase activity, RNA-dependent DNA polymerase activity, RNA-dependent RNA polymerase activity, etc), recombinational activity (e.g., recombination proteins such as Int, IHF, Fis, X is, etc), topoisomerase activity, ligase activity, restriction enzyme activity, β-lactamase activity, β-glucuronidase activity, and the like.

Peptides of the invention may be located at any position in a fusion protein of the invention. For example, peptides of the invention may be located, for example, (1) at the N-terminus, (2) at the C-terminus, (3) at both the N-terminus and C-terminus of the protein, (4) at an internal position of the protein, or combinations thereof. A peptide of the invention may also be located internally (e.g., between regions of amino acid sequence derived from different proteins or different domains of the same protein) and may be attached to an amino acid side chain. For example, Ferguson et al., Protein Sci. 7:1636-1638 (1998), describe a siderophore receptor, FhuA, from Escherichia coli into which an affinity peptide was inserted. This peptide was shown to function in purification protocols employing metal chelate affinity chromatography. Additional fusion proteins with internal tags are described in U.S. Pat. No. 6,143,524, the entire disclosure of which is incorporated herein by reference.

One skilled in the art will recognize that an N-terminal methionine may be post-translationally modified and peptides comprising these post-translational modifications are within the scope of the present invention. For example, a fusion protein comprising a peptide of the invention located at the N-terminal of the fusion protein and comprising an N-terminal methionine may be post-translationally modified and the modified fusion protein is within the scope of the invention. For example, an N-terminal methionine may be cleaved from a fusion protein of the invention, or may be covalently modified (e.g., myristylated, etc.) and the modified fusion protein would be within the scope of the invention.

Peptides of the invention may vary in length but will typically be from about 5 to about 500, from about 5 to about 100, from about 10 to about 100, from about 15 to about 100, from about 20 to about 100, from about 25 to about 100, from about 30 to about 100 from about 35 to about 100, from about 40 to about 100, from about 45 to about 100, from about 50 to about 100, from about 55 to about 100, from about 60 to about 100, from about 65 to about 100, from about 70 to about 100, from about 75 to about 100, from about 80 to about 100, from about 85 to about 100, from about 90 to about 100, from about 95 to about 100, from about 5 to about 80, from about 10 to about 80, from about 20 to about 80, from about 30 to about 80, from about 40 to about 80, from about 50 to about 80, from about 60 to about 80, from about 70 to about 80, from about 5 to about 60, from about 10 to about 60, from about 20 to about 60, from about 30 to about 60, from about 40 to about 60, from about 50 to about 60, from about 5 to about 40, from about 10 to about 40, from about 20 to about 40, from about 30 to about 40, from about 5 to about 30, from about 10 to about 30, from about 20 to about 30, from about 5 to about 25, from about 10 to about 25, or from about 15 to about 25 amino acid residues in length.

In some embodiments, peptides of the invention may bind to immobilized metal ions. Such peptides may be used, for example, with the commonly used IDA resin in IMAC for purification of fusion proteins by virtue of affinity peptides of the invention for the immobilized metal ion.

Peptides of the invention may be attached, covalently or non-covalently, to molecules of interest other than protein molecules. For example, peptides of the invention can be attached to reporter molecules (e.g., fluorophores, chromophores, radiolabels, enzymes and the like). Peptides comprising reporter molecules may optionally be attached to additional molecules (e.g., proteins, nucleic acid molecules, etc.) using techniques well known in the art.

Specific Examples of Peptides of the Invention.

In one aspect of the invention, the present invention provides affinity peptides of the general formula U₁XJYU₂ (SEQ ID NO: 11), wherein, U₁ and U₂ are amino acids independently selected from a group consisting of H, K, or R (histidine, lysine, or arginine), X can be from 1 to 20 amino acid residues, each residue independently drawn from the set of the 20 naturally occurring amino acids commonly found in proteins in either the L or D form of chiral amino acids or a modified amino acid with the proviso that when U₁ is histidine the amino acid of X adjacent to U₁ is not histidine, Y can be from 1 to 20 amino acid residues, each residue independently drawn from the set of the 20 naturally occurring amino acids commonly found in proteins, in either the L or D form of chiral amino acids or Y can be a modified amino acid with the proviso that when U₂ is histidine the amino acid of Y that is adjacent to U₂ is not histidine; and J is drawn from the set: D, E, M, or C (aspartic acid, glutamic acid, methionine, or cysteine). Examples of such peptides are found in Tables 1-6. X and Y may be independently selected, for example, X and Y may be contain the same number of amino acids, which may be the same or different and may be in the same or different order. Alternatively, X and Y may contain a different number of amino acids and/or different amino acids. In some embodiments, X=Y, while in other embodiments, X≠Y. Affinity peptides of this type may be incorporated into fusion proteins, for example, at the N-terminal, C-terminal, and/or at an internal location of the fusion protein. Thus U₁ and/or U₂ may be attached (e.g., via a peptide bond) to a protein sequence of interest.

In a specific example of peptides of the general formula U₁XJYU₂ (SEQ ID NO: 11) suitable for use in the present invention, X and Y may be a single amino acid and may be the same amino acid. For example, both X and Y may designate a single glycine (X=G, Y=G). The following Table provides peptides of the invention that meet this criteria.

TABLE 1

| U₁ U2 | | | | |
|---|---|---|---|---|
| ↓ ↓ | J = D | J = E | J = C | J = M |
| HxxxH | HGDGH | HGEGH | HGCGH | HGMGH |
| (SEQ ID NO: 34) | (SEQ ID NO: 43) | (SEQ ID NO: 52) | (SEQ ID NO: 61) | (SEQ ID NO: 70) |
| HxxxK | HGDGK | HGEGK | HGCGK | HGMGK |
| (SEQ ID NO: 35) | (SEQ ID NO: 44) | (SEQ ID NO: 53) | (SEQ ID NO: 62) | (SEQ ID NO: 71) |
| HxxxR | HGDGR | HGEGR | HGCGR | HGMGR |
| (SEQ ID NO: 36) | (SEQ ID NO: 45) | (SEQ ID NO: 54) | (SEQ ID NO: 63) | (SEQ ID NO: 72) |
| KxxxK | KGDGK | KGEGK | KGCGK | KGMGK |
| (SEQ ID NO: 37) | (SEQ ID NO: 46) | (SEQ ID NO: 55) | (SEQ ID NO: 64) | (SEQ ID NO: 73) |
| KxxxH | KGDGH | KGEGH | KGCGH | KGMGH |
| (SEQ ID NO: 38) | (SEQ ID NO: 47) | (SEQ ID NO: 56) | (SEQ ID NO: 65) | (SEQ ID NO: 74) |
| KxxxR | KGDGR | KGEGR | KGCGR | KGMGR |
| (SEQ ID NO: 39) | (SEQ ID NO: 48) | (SEQ ID NO: 57) | (SEQ ID NO: 66) | (SEQ ID NO: 75) |
| RxxxR | RGDGR | RGEGR | RGCGR | RGMGR |
| (SEQ ID NO: 40) | (SEQ ID NO: 49) | (SEQ ID NO: 58) | (SEQ ID NO: 67) | (SEQ ID NO: 76) |
| RxxxH | RGDGH | RGEGH | RGCGH | RGMGH |
| (SEQ ID NO: 41) | (SEQ ID NO: 50) | (SEQ ID NO: 59) | (SEQ ID NO: 68) | (SEQ ID NO: 77) |
| RxxxK | RGDGK | RGEGK | RGCGK | RGMGK |
| (SEQ ID NO: 42) | (SEQ ID NO: 51) | (SEQ ID NO: 60) | (SEQ ID NO: 69) | (SEQ ID NO: 78) |

In a specific example of peptides of the general formula U₁XJYU₂ (SEQ ID NO: 11) suitable for use in the present invention, X and Y may be two amino acids and may be the same amino acid. For example, X and Y may be two glycines (X=GG, Y=GG). The following Table provides examples of peptides of the invention that meet this criteria.

TABLE 2

| U₁ U2 | | | |
|---|---|---|---|
| ↓ ↓ | J = D | J = E | J = C |
| HxxxxxH | HGGDGGH | HGGEGGH | HGGCGGH |
| (SEQ ID NO: 79) | (SEQ ID NO: 88) | (SEQ ID NO: 97) | (SEQ ID NO: 106) |
| HxxxxxK | HGGDGGK | HGGEGGK | HGGCGGK |
| (SEQ ID NO: 80) | (SEQ ID NO: 89) | (SEQ ID NO: 98) | (SEQ ID NO: 107) |
| HxxxxxR | HGGDGGR | HGGEGGR | HGGCGGR |
| (SEQ ID NO: 81) | (SEQ ID NO: 90) | (SEQ ID NO: 99) | (SEQ ID NO: 108) |
| KxxxxxK | KGGDGGK | KGGEGGK | KGGCGGK |
| (SEQ ID NO: 82) | (SEQ ID NO: 91) | (SEQ ID NO: 100) | (SEQ ID NO: 109) |
| KxxxxxH | KGGDGGH | KGGEGGH | KGGCGGH |
| (SEQ ID NO: 83) | (SEQ ID NO: 92) | (SEQ ID NO: 101) | (SEQ ID NO: 110) |
| KxxxxxR | KGGDGGR | KGGEGGR | KGGCGGR |
| (SEQ ID NO: 84) | (SEQ ID NO: 93) | (SEQ ID NO: 102) | (SEQ ID NO: 111) |
| RxxxxxR | RGGDGGR | RGGEGGR | RGGCGGR |
| (SEQ ID NO: 85) | (SEQ ID NO: 94) | (SEQ ID NO: 103) | (SEQ ID NO: 112) |
| RxxxxxH | RGGDGGH | RGGEGGH | RGGCGGH |
| (SEQ ID NO: 86) | (SEQ ID NO: 95) | (SEQ ID NO: 104) | (SEQ ID NO: 113) |
| RxxxxxK | RGGDGGK | RGGEGGK | RGGCGGK |
| (SEQ ID NO: 87) | (SEQ ID NO: 96) | (SEQ ID NO: 105) | (SEQ ID NO: 114) |

In a specific example of peptides of the general formula U₁XJYU₂ (SEQ ID NO: 11) suitable for use in the present invention, X and Y may be two amino acids and may be different amino acids. For example, X and Y may be a glycine and a serine (X=GS, Y=GS). The following Table provides examples of peptides of the invention that meet this criteria.

TABLE 3

| U₁ U2 | | | |
|---|---|---|---|
| ↓ ↓ | J = D | J = E | J = C |
| HxxxxxH | HGSDGSH | HGSEGSH | HGSCGSH |
| (SEQ ID NO: 79) | (SEQ ID NO: 115) | (SEQ ID NO: 124) | (SEQ ID NO: 133) |
| HxxxxxK | HGSDGSK | HGSEGSK | HGSCGSK |
| (SEQ ID NO: 80) | (SEQ ID NO: 116) | (SEQ ID NO: 125) | (SEQ ID NO: 134) |
| HxxxxxR | HGSDGSR | HGSEGSR | HGSCGSR |
| (SEQ ID NO: 81) | (SEQ ID NO: 117) | (SEQ ID NO: 126) | (SEQ ID NO: 135) |
| KxxxxxK | KGSDGSK | KGSEGSK | KGSCGSK |
| (SEQ ID NO: 82) | (SEQ ID NO: 118) | (SEQ ID NO: 127) | (SEQ ID NO: 136) |
| KxxxxxH | KGSDGSH | KGSEGSH | KGSCGSH |
| (SEQ ID NO: 83) | (SEQ ID NO: 119) | (SEQ ID NO: 128) | (SEQ ID NO: 137) |
| KxxxxxR | KGSDGSR | KGSEGSR | KGSCGSR |
| (SEQ ID NO: 84) | (SEQ ID NO: 120) | (SEQ ID NO: 129) | (SEQ ID NO: 138) |
| RxxxxxR | RGSDGSR | RGSEGSR | RGSCGSR |
| (SEQ ID NO: 85) | (SEQ ID NO: 121) | (SEQ ID NO: 130) | (SEQ ID NO: 139) |
| RxxxxxH | RGSDGSH | RGSEGSH | RGSCGSH |
| (SEQ ID NO: 86) | (SEQ ID NO: 122) | (SEQ ID NO: 131) | (SEQ ID NO: 140) |
| RxxxxxK | RGSDGSK | RGSEGSK | RGSCGSK |
| (SEQ ID NO: 87) | (SEQ ID NO: 123) | (SEQ ID NO: 132) | (SEQ ID NO: 141) |

In a specific example of peptides of the general formula U₁XJYU₂. (SEQ ID NO: 11) suitable for use in the present invention, X and Y may be two amino acids and may be different amino acids. For example, X and Y may be a serine and a glycine (X=SG, Y=SG). The following Table provides examples of peptides of the invention that meet this criteria. Table 4.

TABLE 4

| U₁ U2 | | | |
|---|---|---|---|
| ↓ ↓ | J = D | J = E | J = C |
| HxxxxxH | HSGDSGH | HSGESGH | HSGCSGH |
| (SEQ ID NO: 79) | (SEQ ID NO: 142) | (SEQ ID NO: 151) | (SEQ ID NO: 160) |
| HxxxxxK | HSGDSGK | HSGESGK | HSGCSGK |
| (SEQ ID NO: 80) | (SEQ ID NO: 143) | (SEQ ID NO: 152) | (SEQ ID NO: 161) |
| HxxxxxR | HSGDSGR | HSGESGR | HSGCSGR |
| (SEQ ID NO: 81) | (SEQ ID NO: 144) | (SEQ ID NO: 153) | (SEQ ID NO: 162) |
| KxxxxxK | KSGDSGK | KSGESGK | KSGCSGK |
| (SEQ ID NO: 82) | (SEQ ID NO: 145) | (SEQ ID NO: 154) | (SEQ ID NO: 163) |
| KxxxxxH | KSGDSGH | KSGESGH | KSGCSGH |
| (SEQ ID NO: 83) | (SEQ ID NO: 146) | (SEQ ID NO: 155) | (SEQ ID NO: 164) |
| KxxxxxR | KSGDSGR | KSGESGR | KSGCSGR |
| (SEQ ID NO: 84) | (SEQ ID NO: 147) | (SEQ ID NO: 156) | (SEQ ID NO: 165) |
| RxxxxxR | RSGDSGR | RSGESGR | RSGCSGR |
| (SEQ ID NO: 85) | (SEQ ID NO: 148) | (SEQ ID NO: 157) | (SEQ ID NO: 166) |
| RxxxxxH | RSGDSGH | RSGESGH | RSGCSGH |
| (SEQ ID NO: 86) | (SEQ ID NO: 149) | (SEQ ID NO: 158) | (SEQ ID NO: 167) |
| RxxxxxK | RSGDSGK | RSGESGK | RSGCSGK |
| (SEQ ID NO: 87) | (SEQ ID NO: 150) | (SEQ ID NO: 159) | (SEQ ID NO: 168) |

Those skilled in the art will appreciate that it is not necessary that X and Y have the same number of amino acids in peptides of the general formula U₁XJYU₂ (SEQ ID NO: 11). For example, for a given peptide, X may be a single amino acid while Y may two, three, four, five, etc. amino acids. Also, when X and Y are the same number of amino acids, X and Y may comprise one or more different amino acids (e.g., X=GS while Y=AQ, etc.).

In a specific example of peptides of the general formula U₁XJYU₂ (SEQ ID NO: 11) suitable for use in the present invention, X and Y may be a single amino acid and may be different amino acids. For example, X may be a glycine and Y may be a serine (X=G, Y=S). The following Table provides examples of peptides of the invention that meet this criteria.

TABLE 5

| U₁ U2 | | | |
|---|---|---|---|
| ↓ ↓ | J = D | J = E | J = C |
| HxxxH | HGDSH | HGESH | HGCSH |
| (SEQ ID NO: 34) | (SEQ ID NO: 169) | (SEQ ID NO: 178) | (SEQ ID NO: 187) |
| HxxxK | HGDSK | HGESK | HGCSK |
| (SEQ ID NO: 35) | (SEQ ID NO: 170) | (SEQ ID NO: 179) | (SEQ ID NO: 188) |
| HxxxR | HGDSR | HGESR | HGCSR |
| (SEQ ID NO: 36) | (SEQ ID NO: 171) | (SEQ ID NO: 180) | (SEQ ID NO: 189) |
| KxxxK | KGDSK | KGESK | KGCSK |
| (SEQ ID NO: 37) | (SEQ ID NO: 172) | (SEQ ID NO: 181) | (SEQ ID NO: 190) |
| KxxxH | KGDSH | KGESH | KGCSH |
| (SEQ ID NO: 38) | (SEQ ID NO: 173) | (SEQ ID NO: 182) | (SEQ ID NO: 191) |
| KxxxR | KGDSR | KGESR | KGCSR |
| (SEQ ID NO: 39) | (SEQ ID NO: 174) | (SEQ ID NO: 183) | (SEQ ID NO: 192) |
| RxxxR | RGDSR | RGESR | RGCSR |
| (SEQ ID NO: 40) | (SEQ ID NO: 175) | (SEQ ID NO: 184) | (SEQ ID NO: 193) |
| RxxxH | RGDSH | RGESH | RGCSH |
| (SEQ ID NO: 41) | (SEQ ID NO: 176) | (SEQ ID NO: 185) | (SEQ ID NO: 194) |
| RxxxK | RGDSK | RGESK | RGCSK |
| (SEQ ID NO: 42) | (SEQ ID NO: 177) | (SEQ ID NO: 186) | (SEQ ID NO: 195) |

In a specific example of peptides of the general formula U₁XJYU₂ (SEQ ID NO: 11) suitable for use in the present invention, X and Y may be a single amino acid and may be different amino acids. For example, X may be a serine and Y may be a glycine (X=S, Y=G). The following Table provides examples of peptides of the invention that meet this criteria.

TABLE 6

| U₁ U2 | | | |
|---|---|---|---|
| ↓ ↓ | J = D | J = E | J = C |
| HxxxH | HSDGH | HSEGH | HSCGH |
| (SEQ ID NO: 34) | (SEQ ID NO: 196) | (SEQ ID NO: 205) | (SEQ ID NO: 214) |
| HxxxK | HSDGK | HSEGK | HSCGK |
| (SEQ ID NO: 35) | (SEQ ID NO: 197) | (SEQ ID NO: 206) | (SEQ ID NO: 215) |
| HxxxR | HSDGR | HSEGR | HSCGR |
| (SEQ ID NO: 36) | (SEQ ID NO: 198) | (SEQ ID NO: 207) | (SEQ ID NO: 216) |
| KxxxK | KSDGK | KSEGK | KSCGK |
| (SEQ ID NO: 37) | (SEQ ID NO: 199) | (SEQ ID NO: 208) | (SEQ ID NO: 217) |
| KxxxH | KSDGH | KSEGH | KSCGH |
| (SEQ ID NO: 38) | (SEQ ID NO: 200) | (SEQ ID NO: 209) | (SEQ ID NO: 218) |
| KxxxR | KSDGR | KSEGR | KSCGR |
| (SEQ ID NO: 39) | (SEQ ID NO: 201) | (SEQ ID NO: 210) | (SEQ ID NO: 219) |
| RxxxR | RSDGR | RSEGR | RSCGR |
| (SEQ ID NO: 40) | (SEQ ID NO: 202) | (SEQ ID NO: 211) | (SEQ ID NO: 220) |
| RxxxH | RSDGH | RSEGH | RSCGH |
| (SEQ ID NO: 41) | (SEQ ID NO: 203) | (SEQ ID NO: 212) | (SEQ ID NO: 221) |
| RxxxK | RSDGK | RSEGK | RSCGK |
| (SEQ ID NO: 42) | (SEQ ID NO: 204) | (SEQ ID NO: 213) | (SEQ ID NO: 222) |

In yet another aspect of the invention, the present invention provides affinity peptides of the general formula J₁X₁UX₂J₂ (SEQ ID NO: 12), wherein J₁ and J₂ are independently drawn from the set: D, E, or C (aspartic acid, glutamic acid, cysteine); X₁ and X₂ are independently from 1 to 20 amino acid residues drawn from the set of the 20 naturally occurring amino acids commonly found in proteins, either the L or D form of chiral amino acids, and X₁ and/or X₂ can be a modified amino acid; U is drawn from the set: H, K, or R (histidine, lysine, arginine), with the proviso that when U is histidine, the amino acids of X₁ and X₂ adjacent to U are not histidine. X₁ and X₂ may be independently selected, for example, X₁ and X₂ may be contain the same number of amino acids, which may be the same or different and may be in the same or different order. Alternatively, X₁ and X₂ may contain a different number of amino acids and/or different amino acids. In some embodiments, X₁=X₂, while in other embodiments, X₁≠X₂. Affinity peptides of this type may be incorporated into fusion proteins, for example, at the N-terminus, C-terminus, and/or at an internal location of the fusion protein. Thus J₁ and/or J₂ may be attached (e.g., via a peptide bond) to a protein sequence of interest. Examples of peptide of this type are provided in Tables 7-10.

In a specific example of peptides of the general formula J₁X₁UX₂J₂ (SEQ ID NO: 12) suitable for use in the present invention X₁ and X₂, may be a single amino acid and may be the same amino acid. For example, X₁ and X₂ may be a single amino acid glycine (X₁=G, X₂=G). The following Table provides peptides of the invention that meet this criteria.

TABLE 7

| J₁ J₂ | | | |
|---|---|---|---|
| ↓ ↓ | U = H | U = K | U = R |
| DxxxD | DGHGD | DGKGD | DGRGD |
| (SEQ ID NO: 223) | (SEQ ID NO: 232) | (SEQ ID NO: 241) | (SEQ ID NO: 250) |
| DxxxE | DGHGE | DGKGE | DGRGE |
| (SEQ ID NO: 224) | (SEQ ID NO: 233) | (SEQ ID NO: 242) | (SEQ ID NO: 251) |
| DxxxC | DGHGC | DGKGC | DGRGC |
| (SEQ ID NO: 225) | (SEQ ID NO: 234) | (SEQ ID NO: 243) | (SEQ ID NO: 252) |
| ExxxD | EGHGD | EGKGD | EGRGD |
| (SEQ ID NO: 226) | (SEQ ID NO: 235) | (SEQ ID NO: 244) | (SEQ ID NO: 253) |
| ExxxE | EGHGE | EGKGE | EGRGE |
| (SEQ ID NO: 227) | (SEQ ID NO: 236) | (SEQ ID NO: 245) | (SEQ ID NO: 254) |
| ExxxC | EGHGC | EGKGC | EGRGC |
| (SEQ ID NO: 228) | (SEQ ID NO: 237) | (SEQ ID NO: 246) | (SEQ ID NO: 255) |
| CxxxD | CGHGD | CGKGD | CGRGD |
| (SEQ ID NO: 229) | (SEQ ID NO: 238) | (SEQ ID NO: 247) | (SEQ ID NO: 256) |
| CxxxE | CGHGE | CGKGE | CGRGE |
| (SEQ ID NO: 230) | (SEQ ID NO: 239) | (SEQ ID NO: 248) | (SEQ ID NO: 257) |
| CxxxC | CGHGC | CGKGC | CGRGC |
| (SEQ ID NO: 231) | (SEQ ID NO: 240) | (SEQ ID NO: 249) | (SEQ ID NO: 258) |

In a specific example of peptides of the general formula J₁X₁UX₂J₂ (SEQ ID NO: 12) suitable for use in the present invention X₁ and X₂, may be the same number of amino acids and may be the same amino acid. For example, X₁ and X₂ may be two glycines (X₁=GG, X₂=GG). The following Table provides peptides of the invention that meet this criteria.

TABLE 8

| J₁ J₂ | | | |
|---|---|---|---|
| ↓ ↓ | U = H | U = K | U = R |
| DxxxD | DGGHGGD | DGGKGGD | DGGRGGD |
| (SEQ ID NO: 259) | (SEQ ID NO: 268) | (SEQ ID NO: 277) | (SEQ ID NO: 286) |
| DxxxE | DGGHGGE | DGGKGGE | DGGRGGE |
| (SEQ ID NO: 260) | (SEQ ID NO: 269) | (SEQ ID NO: 278) | (SEQ ID NO: 287) |
| DxxxC | DGGHGGC | DGGKGGC | DGGRGGC |
| (SEQ ID NO: 261) | (SEQ ID NO: 270) | (SEQ ID NO: 279) | (SEQ ID NO: 288) |
| ExxxD | EGGHGGD | EGGKGGD | EGGRGGD |
| (SEQ ID NO: 262) | (SEQ ID NO: 271) | (SEQ ID NO: 280) | (SEQ ID NO: 289) |
| ExxxE | EGGHGGE | EGGKGGE | EGGRGGE |
| (SEQ ID NO: 263) | (SEQ ID NO: 272) | (SEQ ID NO: 281) | (SEQ ID NO: 290) |
| ExxxC | EGGHGGC | EGGKGGC | EGGRGGC |
| (SEQ ID NO: 264) | (SEQ ID NO: 273) | (SEQ ID NO: 282) | (SEQ ID NO: 291) |
| CxxxD | CGGHGGD | CGGKGGD | CGGRGGD |
| (SEQ ID NO: 265) | (SEQ ID NO: 274) | (SEQ ID NO: 283) | (SEQ ID NO: 292) |
| CxxxE | CGGHGGE | CGGKGGE | CGGRGGE |
| (SEQ ID NO: 266) | (SEQ ID NO: 275) | (SEQ ID NO: 284) | (SEQ ID NO: 293) |
| CxxxC | CGGHGGC | CGGKGGC | CGGRGGC |
| (SEQ ID NO: 267) | (SEQ ID NO: 276) | (SEQ ID NO: 285) | (SEQ ID NO: 294) |

In a specific example of peptides of the general formula J₁X₁UX₂J₂ (SEQ ID NO: 12) suitable for use in the present invention X₁ and X₂, may be a single amino acid and may be different amino acids. For example, X₁ may be glycine and X₂ may be serine (X₁=G, X₂=S). The following Table provides peptides of the invention that meet this criteria.

TABLE 9

| J₁ J₂ | | | |
|---|---|---|---|
| ↓ ↓ | U = H | U = K | U = R |
| DxxxD | DGHSD | DGKSD | DGRSD |
| (SEQ ID NO: 259) | (SEQ ID NO: 295) | (SEQ ID NO: 305) | (SEQ ID NO: 314) |
| DxxxE | DGHSE | DGKSE | DGRSE |
| (SEQ ID NO: 260) | (SEQ ID NO: 296) | (SEQ ID NO: 306) | (SEQ ID NO: 315) |
| DxxxC | DGHSC | DGKSC | DGRSC |
| (SEQ ID NO: 261) | (SEQ ID NO: 297) | (SEQ ID NO: 307) | (SEQ ID NO: 316) |
| ExxxD | EGHSD | EGKSD | EGRSD |
| (SEQ ID NO: 262) | (SEQ ID NO: 298) | (SEQ ID NO: 308) | (SEQ ID NO: 317) |
| ExxxE | EGHSE | EGKSE | EGRSE |
| (SEQ ID NO: 263) | (SEQ ID NO: 299) | (SEQ ID NO: 309) | (SEQ ID NO: 318) |
| | EGHGE | | |
| | (SEQ ID NO: 300) | | |
| ExxxC | EGHSC | EGKSC | EGRSC |
| (SEQ ID NO: 264) | (SEQ ID NO: 301) | (SEQ ID NO: 310) | (SEQ ID NO: 319) |
| CxxxD | CGHSD | CGKSD | CGRSD |
| (SEQ ID NO: 265) | (SEQ ID NO: 302) | (SEQ ID NO: 311) | (SEQ ID NO: 320) |
| CxxxE | CGHSE | CGKSE | CGRSE |
| (SEQ ID NO: 266) | (SEQ ID NO: 303) | (SEQ ID NO: 312) | (SEQ ID NO: 321) |
| CxxxC | CGHSC | CGKSC | CGRGC |
| (SEQ ID NO: 267) | (SEQ ID NO: 304) | (SEQ ID NO: 313) | (SEQ ID NO: 322) |

In a specific example of peptides of the general formula J₁X₁UX₂J₂ (SEQ ID NO: 12) suitable for use in the present invention X₁ and X₂, may be two amino acids and may be different amino acids. For example, X₁ may be two glycines and X₂ may two serines (X₁=GG, X₂=SS). The following Table provides peptides of the invention that meet this criteria.

TABLE 10

| J₁ J₂ | | | |
|---|---|---|---|
| ↓ ↓ | U = H | U = K | U = R |
| DxxxD | DGGHSSD | DGGKSSD | DGGRSSD |
| (SEQ ID NO: 259) | (SEQ ID NO: 323) | (SEQ ID NO: 332) | (SEQ ID NO: 341) |
| DxxxE | DGGHSSE | DGGKSSE | DGGRSSE |
| (SEQ ID NO: 260) | (SEQ ID NO: 324) | (SEQ ID NO: 333) | (SEQ ID NO: 342) |
| DxxxC | DGGHSSC | DGGKSSC | DGGRSSC |
| (SEQ ID NO: 261) | (SEQ ID NO: 325) | (SEQ ID NO: 334) | (SEQ ID NO: 343) |
| ExxxD | EGGHSSD | EGGKSSD | EGGRSSD |
| (SEQ ID NO: 262) | (SEQ ID NO: 326) | (SEQ ID NO: 335) | (SEQ ID NO: 344) |
| ExxxE | EGGHSSE | EGGKSSE | EGGRSSE |
| (SEQ ID NO: 263) | (SEQ ID NO: 327) | (SEQ ID NO: 336) | (SEQ ID NO: 345) |
| ExxxC | EGGHSSC | EGGKSSC | EGGRSSC |
| (SEQ ID NO: 264) | (SEQ ID NO: 328) | (SEQ ID NO: 337) | (SEQ ID NO: 346) |
| CxxxD | CGGHSSD | CGGKSSD | CGGRSSD |
| (SEQ ID NO: 265) | (SEQ ID NO: 329) | (SEQ ID NO: 338) | (SEQ ID NO: 347) |
| CxxxE | CGGHSSE | CGGKSSE | CGGRSSE |
| (SEQ ID NO: 266) | (SEQ ID NO: 330) | (SEQ ID NO: 339) | (SEQ ID NO: 348) |
| CxxxC | CGGHSSC | CGGKSSC | CGGRSSC |
| (SEQ ID NO: 267) | (SEQ ID NO: 331) | (SEQ ID NO: 340) | (SEQ ID NO: 349) |

Those skilled in the art will appreciate that, for peptides of the general formula J₁X₁UX₂J₂ (SEQ ID NO: 12), it is not necessary that X₁ and X₂ have the same number of amino acids. For example, for a given peptide, X₁ may be a single amino acid while X₂ may two, three, four, five, etc. amino acids. Also, when X₁ and X₂ are the same number of amino acids, X₁ and X₂ may comprise one or more different amino acids (e.g., X₁=GS while X₂=AQ, etc.).

In another aspect of the invention, the present invention provides affinity peptides of the general formula H(XᵢH)ⱼ (SEQ ID NO: 13) where i=1-6 and j=1-6, with the proviso that when j≧2, at least one pair of Xᵢ adjacent to the same histidine do not have the same number of amino acids. Each Xᵢ may independently be from 1 to 6 amino acid residues drawn from the set of the 20 naturally occurring amino acids commonly found in proteins in either the L or D form of chiral amino acids or a modified amino acid with the proviso that the amino acid of X adjacent to H is not histidine. Affinity peptides of this type may be incorporated into fusion proteins, for example, at the N-terminal, C-terminal, and/or at an internal location of the fusion protein. Thus, the N-terminal histidine and/or the C-terminal histidine may be attached (e.g., via a peptide bond) to a protein sequence of interest.

In some embodiments, each Xᵢ is a single amino acid and is the same amino acid. Examples of this are provided in the following Table 11 for the case where j=2.

TABLE 11
j = 2, each Xᵢ same single AA

HDHDH (SEQ ID NO: 350)
HEHEH (SEQ ID NO: 351)
HSHSH (SEQ ID NO: 352)
HTHTH (SEQ ID NO: 353)
HNHNH (SEQ ID NO: 354)
HQHQH (SEQ ID NO: 355)
HPHPH (SEQ ID NO: 356)
HGHGH (SEQ ID NO: 357)
HAHAH (SEQ ID NO: 358)
HKHKH (SEQ ID NO: 359)
HRHRH (SEQ ID NO: 360)
HYHYH (SEQ ID NO: 361)
HMHMH (SEQ ID NO: 362)

In other embodiments of peptides of the general formula H(XᵢH)ⱼ (SEQ ID NO: 13), each Xᵢ need not contain the same number of amino acids and/or need not contain the same amino acids. For example, for the case where j=2, a first Xᵢ might contain one amino acid and a second Xᵢ might contain two amino acids. The amino acid in the first Xᵢ may be the same or different from the amino acids in the second Xᵢ and the amino acids in the second Xᵢ may be the same or different from each other. Alternatively, a first Xᵢ might contain two amino acids and a second Xᵢ might contain one amino acid. The amino acid in the second Xᵢ may be the same or different from the amino acids in the first Xᵢ and the amino acids in the first Xᵢ may be the same or different from each other. Examples of peptides of this type include, but are not limited to HGAHGH (SEQ ID NO: 363), HGAH (SEQ ID NO: 364), AHVH (SEQ ID NO: 365), HDDH (SEQ ID NO: 366), HDDHDH (SEQ ID NO: 367).

In yet another aspect of the invention, the present invention provides affinity peptides with the general formula aHbHc (SEQ ID NO: 14), wherein H is histidine; a=zero or more amino acids, drawn from the set of the 20 naturally occurring amino acids commonly found in proteins in either the L or D form of chiral amino acids or a modified amino acid with the proviso that the amino acid of a adjacent to H is not histidine; b=one or more amino acids, drawn from the set of the 20 naturally occurring amino acids commonly found in proteins in either the L or D form of chiral amino acids or a modified amino acid with the proviso that the amino acid of b adjacent to H is not histidine; and c=zero or more amino acids, drawn from the set of the 20 naturally occurring amino acids commonly found in proteins in either the L or D form of chiral amino acids or a modified amino acid with the proviso that the amino acid of c adjacent to H is not histidine. Affinity peptides of this type may be incorporated into fusion proteins, for example, at the N-terminus, C-terminus, and/or at an internal location of the fusion protein. Thus a and/or c may be attached (e.g., via a peptide bond) to a protein sequence of interest.

In a specific example of embodiments of peptides with the general formula aHbHc (SEQ ID NO: 14), a, b, and c may be single amino acids, which may be the same or different. In the first column of Table 12, examples are provided of the case where a, b, and c are each a single amino acid and they are the same amino (i.e., a=b=c). In some embodiments, one or more of a, b, and c may be multiple amino acids (e.g., two, three, four, five, six, seven, eight, nine, ten, etc.). In embodiments of this type, the same or different amino acids may be used for the multiple amino acids and these may be the same or different as the amino acids that are single amino acids. The second column of Table 12 shows the case where a and c are single amino acids and are the same amino acid, and b is two amino acids that are the same and are the same as the amino acids of a and c.

TABLE 12

| a, b, and c are single amino | a and c are single amino acids |
|---|---|
| acids and a = b = c | and b is two amino acids |
| GHGHG (SEQ ID NO: 367) | GHGGHG (SEQ ID NO: 371) |
| AHAHA (SEQ ID NO: 368) | AHAAHA (SEQ ID NO: 372) |
| VHVHV (SEQ ID NO: 369) | VHVVHV (SEQ ID NO: 373) |
| LHLHL (SEQ ID NO: 370) | LHLLHL (SEQ ID NO: 374) |
| IHIHI (SEQ ID NO: 375) | IHIIHI (SEQ ID NO: 390) |
| SHSHS (SEQ ID NO: 376) | SHSSHS (SEQ ID NO: 391) |
| THTHT (SEQ ID NO: 377) | THTTHT (SEQ ID NO: 392) |
| NHNHN (SEQ ID NO: 378) | NHNNHN (SEQ ID NO: 393) |
| QHQHQ (SEQ ID NO: 379) | QHQQHQ (SEQ ID NO: 394) |
| KHKHK (SEQ ID NO: 380) | KHKKHK (SEQ ID NO: 395) |
| RHRHR (SEQ ID NO: 381) | RHRRHR (SEQ ID NO: 396) |
| DHDHD (SEQ ID NO: 382) | DHDDHD (SEQ ID NO: 397) |
| EHEHE (SEQ ID NO: 383) | EHEEHE (SEQ ID NO: 398) |
| CHCHC (SEQ ID NO: 384) | CHCCHC (SEQ ID NO: 399) |
| MHMHM (SEQ ID NO: 385) | MHMMHM (SEQ ID NO: 400) |
| FHFHF (SEQ ID NO: 386) | FHFFHF (SEQ ID NO: 401) |
| WHWHW (SEQ ID NO: 387) | WHWWHW (SEQ ID NO: 402) |
| YHYHY (SEQ ID NO: 388) | YHYYHY (SEQ ID NO: 403) |
| PHPHP (SEQ ID NO: 389) | PHPPHP (SEQ ID NO: 404) |

Other specific examples of peptides with the general formula aHbHc (SEQ ID NO: 14), include the case where a, b, and c are single amino acids and one of a, b, or c (in the case shown c) is different from the other two. The first column of Table 13 shows the case where a and b are the same and c is different. In this case, c is a single amino acid (indicated by the subscript c₁) and may be any non-histidine amino acid that is different than a and b. The second column of Table 13 shows the case where one of the variables (in the case shown b) indicates two amino acids (i.e., b₁b₂) and the other two (i.e., a and c) indicate single amino acids. In the case shown in the table, the first amino acid of b is the same as a and c and the second amino acid of b (indicated by the subscript b₂) is different. In this case, b₂ may be any non-histidine amino acid that is different than a, c and b₁.

TABLE 13

| a = b ≠ c | a = b₁ = c ≠ b₂ |
|---|---|
| GHGHc₁ (SEQ ID NO: 405) | GHGb₂HG (SEQ ID NO: 424) |
| AHAHc₁ (SEQ ID NO: 406) | AHAb₂HA (SEQ ID NO: 425) |
| VHVHc₁ (SEQ ID NO: 407) | VHVb₂HV (SEQ ID NO: 426) |
| LHLHc₁ (SEQ ID NO: 408) | LHLb₂HL (SEQ ID NO: 427) |
| IHIHIc₁ (SEQ ID NO: 409) | IHIb₂HI (SEQ ID NO: 428) |
| SHSHc₁ (SEQ ID NO: 410) | SHSb₂HS (SEQ ID NO: 429) |
| THTHc₁ (SEQ ID NO: 411) | THTb₂HT (SEQ ID NO: 430) |
| NHNHc₁ (SEQ ID NO: 412) | NHNb₂HN (SEQ ID NO: 431) |
| QHQHc₁ (SEQ ID NO: 413) | QHQb₂HQ (SEQ ID NO: 432) |
| KHKHc₁ (SEQ ID NO: 414) | KHKb₂HK (SEQ ID NO: 433) |
| RHRHc₁ (SEQ ID NO: 415) | RHRb₂HR (SEQ ID NO: 434) |
| DHDHc₁ (SEQ ID NO: 416) | DHDb₂HD (SEQ ID NO: 435) |
| EHEHc₁ (SEQ ID NO: 417) | EHEb₂HE (SEQ ID NO: 436) |
| CHCHc₁ (SEQ ID NO: 418) | CHCb₂HC (SEQ ID NO: 437) |
| MHMHc₁ (SEQ ID NO: 419) | MHMb₂HM (SEQ ID NO: 438) |
| FHFHc₁ (SEQ ID NO: 420) | FHFb₂HF (SEQ ID NO: 439) |
| WHWHc₁ (SEQ ID NO: 421) | WHWb₂HW (SEQ ID NO: 440) |
| YHYHc₁ (SEQ ID NO: 422) | YHYb₂HY (SEQ ID NO: 441) |
| PHPHc₁ (SEQ ID NO: 423) | PHPb₂HP (SEQ ID NO: 442) |

Other specific examples of peptides with the general formula aHbHc (SEQ ID NO: 14), include the case where one of the variables (in the case shown b) indicates multiple amino acids (i.e., b₁b₂) and the other two (i.e., a and c) indicate single amino acids and one of the single amino acids is different from the other single amino acid. In embodiments of this type, one or more of the multiple amino acids may be the same as either of the single amino acids. Examples of this type are shown in Tables 14 and 15. The first row of each table shows examples of the case where all of the multiple amino acids are the same as one of the single amino acids (i.e., a≠b1=b2=c). In the remaining rows, examples of the case where only one of the multiple amino acids is the same as one of the single amino acids is shown (i.e., a≠b1, a≠b2, b1≠b2, b1=c).

TABLE 14
aHb₁b₂Hc (SEQ ID NO: 691)

| MHDDHD | MHEEHE | MHSSHS | MHTTHT | MHNNHN | MHQQHQ |
|---|---|---|---|---|---|
| (SEQ ID NO: | (SEQ ID NO: | (SEQ ID NO: | (SEQ ID NO: | (SEQ ID NO: | (SEQ ID NO: |
| 443) | 455) | 467) | 479) | 491) | 503) |
| MHDEHD | MHEDHE | MHSDHS | MHTEHT | MHNDHN | MHQDHQ |
| (SEQ ID NO: | (SEQ ID NO: | (SEQ ID NO: | (SEQ ID NO: | (SEQ ID NO: | (SEQ ID NO: |
| 444) | 456) | 468) | 480) | 492) | 504) |
| MHDSHD | MHESHE | MHSEHS | MHTSHT | MHNEHN | MHQEHQ |
| (SEQ ID NO: | (SEQ ID NO: | (SEQ ID NO: | (SEQ ID NO: | (SEQ ID NO: | (SEQ ID NO: |
| 445) | 457) | 469) | 481) | 493) | 505) |
| MHDTHD | MHETHE | MHSTHS | MHTDHT | MHNSHN | MHQSHQ |
| (SEQ ID NO: | (SEQ ID NO: | (SEQ ID NO: | (SEQ ID NO: | (SEQ ID NO: | (SEQ ID NO: |
| 446) | 458) | 470) | 482) | 494) | 506) |
| MHDNHD | MHENHE | MHSNHS | MHTNHT | MHNTHN | MHQTHQ |
| (SEQ ID NO: | (SEQ ID NO: | (SEQ ID NO: | (SEQ ID NO: | (SEQ ID NO: | (SEQ ID NO: |
| 447) | 459) | 471) | 483) | 495) | 507) |
| MHDQHD | MHEQHE | MHSQHS | MHTQHT | MHNQHN | MHQNHQ |
| (SEQ ID NO: | (SEQ ID NO: | (SEQ ID NO: | (SEQ ID NO: | (SEQ ID NO: | (SEQ ID NO: |
| 448) | 460) | 472) | 484) | 496) | 508) |
| MHDPHD | MHEPHE | MHSPHS | MHTPHT | MHNPHN | MHQPHQ |
| (SEQ ID NO: | (SEQ ID NO: | (SEQ ID NO: | (SEQ ID NO: | (SEQ ID NO: | (SEQ ID NO: |
| 449) | 461) | 473) | 485) | 497) | 509) |
| MHDGHD | MHEGHE | MHSGHS | MHTGHT | MHNGHN | MHQGHQ |
| (SEQ ID NO: | (SEQ ID NO: | (SEQ ID NO: | (SEQ ID NO: | (SEQ ID NO: | (SEQ ID NO: |
| 450) | 462) | 474) | 486) | 498) | 510) |
| MHDAHD | MHEAHE | MHSAHS | MHTAHT | MHNAHN | MHQAHQ |
| (SEQ ID NO: | (SEQ ID NO: | (SEQ ID NO: | (SEQ ID NO: | (SEQ ID NO: | (SEQ ID NO: |
| 451) | 463 | 475) | 487) | 499) | 511) |
| MHDKHD | MHEKHE | MHSKHS | MHTKHT | MHNKHN | MHQKHQ |
| (SEQ ID NO: | (SEQ ID NO: | (SEQ ID NO: | (SEQ ID NO: | (SEQ ID NO: | (SEQ ID NO: |
| 452) | 464) | 476) | 488) | 500) | 512) |
| MHDRHD | MHERHE | MHSRHS | MHTRHT | MHNRHN | MHQRHQ |
| (SEQ ID NO: | (SEQ ID NO: | (SEQ ID NO: | (SEQ ID NO: | (SEQ ID NO: | (SEQ ID NO: |
| 453) | 465) | 477) | 489) | 501) | 513) |
| MHDYHD | MHEYHE | MUSYHS | MHTYHT | MHNYHN | MHQHYQ |
| (SEQ ID NO: | (SEQ ID NO: | (SEQ ID NO: | (SEQ ID NO: | (SEQ ID NO: | (SEQ ID NO: |
| 454) | 466) | 478) | 490) | 502) | 514) |

TABLE 15
aHb₁b₂Hc (SEQ ID NO: 691)

| MHPPHP | MHGGHG | MHAAHA | MHKKHK | MHRRHR | MHYYHY |
|---|---|---|---|---|---|
| (SEQ ID NO: | (SEQ ID NO: | (SEQ ID NO: | (SEQ ID NO: | (SEQ ID NO: | (SEQ ID NO: |
| 515) | 527) | 539) | 551) | 563) | 575) |
| MHPDHP | MHGDHG | MHADHA | MHKDHK | MHRDHR | MHYDHY |
| (SEQ ID NO: | (SEQ ID NO: | (SEQ ID NO: | (SEQ ID NO: | (SEQ ID NO: | (SEQ ID NO: |
| 516) | 528) | 540) | 552) | 564) | 576) |
| MHPEHP | MHGEHG | MHAEHA | MHKEHK | MHREHR | MHYEHY |
| (SEQ ID NO: | (SEQ ID NO: | (SEQ ID NO: | (SEQ ID NO: | (SEQ ID NO: | (SEQ ID NO: |
| 517) | 529) | 541) | 553) | 565) | 577) |
| MHPSHP | MHGSHG | MHASHA | MHKSHK | MHRSHR | MHYSHY |
| (SEQ ID NO: | (SEQ ID NO: | (SEQ ID NO: | (SEQ ID NO: | (SEQ ID NO: | (SEQ ID NO: |
| 518) | 530) | 542) | 554) | 566) | 578) |
| MHPTHP | MHGTHG | MHATHA | MHKTHK | MHRTHR | MHYTHY |
| (SEQ ID NO: | (SEQ ID NO: | (SEQ ID NO: | (SEQ ID NO: | (SEQ ID NO: | (SEQ ID NO: |
| 519) | 531) | 543) | 555) | 567) | 579) |
| MHPNHP | MHGNHG | MHANHA | MHKNHK | MHRNHR | MHYNHY |
| (SEQ ID NO: | (SEQ ID NO: | (SEQ ID NO: | (SEQ ID NO: | (SEQ ID NO: | (SEQ ID NO: |
| 520) | 532) | 544) | 556) | 568) | 580) |
| MHPQHP | MHGQHG | MHAQHA | MHKQHK | MHRQHR | MHYQHY |
| (SEQ ID NO: | (SEQ ID NO: | (SEQ ID NO: | (SEQ ID NO: | (SEQ ID NO: | (SEQ ID NO: |
| 521) | 533) | 545) | 557) | 569) | 581) |
| MHPGHP | MHGPHG | MHAPHA | MHKPHK | MHRPHR | MHYPHY |
| (SEQ ID NO: | (SEQ ID NO: | (SEQ ID NO: | (SEQ ID NO: | (SEQ ID NO: | (SEQ ID NO: |
| 522) | 534) | 546) | 558) | 570) | 582) |
| MHPAHP | MHGAHG | MHAGHA | MHKGHK | MHRGHR | MHYGHY |
| (SEQ ID NO: | (SEQ ID NO: | (SEQ ID NO: | (SEQ ID NO: | (SEQ ID NO: | (SEQ ID NO: |
| 523) | 535) | 547) | 559) | 571) | 583) |
| MHPKHP | MHGKHG | MHAKHA | MHKAHK | MHRAHR | MHYAHY |
| (SEQ ID NO: | (SEQ ID NO: | (SEQ ID NO: | (SEQ ID NO: | (SEQ ID NO: | (SEQ ID NO: |
| 524) | 536) | 548) | 560) | 572) | 584) |
| MHPRHP | MHGRHG | MHARHA | MHKRHK | MHRKHR | MHYKHY |
| (SEQ ID NO: | (SEQ ID NO: | (SEQ ID NO: | (SEQ ID NO: | (SEQ ID NO: | (SEQ ID NO: |
| 525) | 537) | 549) | 561) | 573) | 585) |
| MHPYHP | MHGYHG | MHAYHA | MHKYHK | MHRYHR | MHYRHY |
| (SEQ ID NO: | (SEQ ID NO: | (SEQ ID NO: | (SEQ ID NO: | (SEQ ID NO: | (SEQ ID NO: |
| 526) | 538) | 550) | 562) | 574) | 586) |

Other specific examples of peptides with the general formula aHbHc (SEQ ID NO: 14), include the case where a designates methionine (a=M). Peptides of this type may be particularly useful as N-terminal peptides. The first two columns of Table 16 provide specific examples of peptides of this type. The peptides in the first two column of Table 16 show the case where a=M and b=c and b and c are single amino acids. The third column of Table 16 provides specific examples of the case where a is not a single amino acid, for example, the first four peptides in column three show the case where a=0 amino acids and the last three peptides show the case where a=2 amino acids and the amino acids are different (a=a₁a₂=MD or GS). In addition, column three shows the case where c=0 amino acids (i.e., the peptides end in histidine). Peptides three and four of column 3 show the case where b is not a single amino acid, for example b=b₁b₂b₃b₄=GAKG (SEQ ID NO: 587) or GARG (SEQ ID NO: 588). The last column of Table 16 shows the case where a is a single amino acid (a=E), b is three amino acids (b=b₁b₂b₃=GMG), and c is two amino acids (c=c₁c₂=NT).

TABLE 16

| | | | |
|---|---|---|---|
| MHYHY | MHRHR | HEH | EHGMGHNT |
| (SEQ ID NO: 589) | (SEQ ID NO: 595) | | (SEQ ID NO: 606) |
| MHQHQ | MHNHN | HGH | |
| (SEQ ID NO: 590) | (SEQ ID NO: 596) | | |
| MHPHP | MHDHD | HGAKGH | |
| (SEQ ID NO: 591) | (SEQ ID NO: 597) | (SEQ ID NO: 601) | |
| MHGHG | MHEHE | HGARGH | |
| (SEQ ID NO: 592) | (SEQ ID NO: 598) | (SEQ ID NO: 602) | |
| MHAHA | MHSHS | MDHDH | |
| (SEQ ID NO: 593) | (SEQ ID NO: 599) | (SEQ ID NO: 603) | |
| MHKHK | MHTHT | GSHDH | |
| (SEQ ID NO: 594) | (SEQ ID NO: 600) | (SEQ ID NO: 604) | |
| | | GSHGH | |
| | | (SEQ ID NO: 605) | |

In another embodiment, the present invention provides a method for identifying a peptide that binds to an immobilized metal ion, such as an immobilized metal ion associated with a chromatography matrix, by identifying a segment in a polypeptide that includes at least 4 histidine residues that make up at least 25% of the segment. The method can be used to isolate a peptide that binds to an immobilized metal ion-containing chromatography matrix. The method includes analyzing the amino acid sequence of a polypeptide, such as a naturally-occurring protein, to identify a segment that include at least 4 histidine residues, wherein the histidine residues make up at least 25% of the amino acids in the segment, and isolating a peptide that includes the segment. The segment can include, for example, 4, 5, 6, 7, 8, 9, 10, 15, or 20 histidine residues. The segment can include, for example, 25, 30, 35, 40, 45, 50, 60, 70, 75, 80, 90, 95 or 100% histidine residues. The segment can be, for example, between 5 and 500 amino acids in length.

It will be understood that many known methods can be used to scan a sequence for segments that contain at least 4 histidine residues that make up at least 25% of the segment. These methods include manual methods as well as automated methods, such as those performed by computer programs. Furthermore, many methods are known for isolating a peptide, including, for example, synthesizing the peptide using an automated synthesizer or synthesizing the peptide in a cell or cell-free extract that includes a nucleic acid molecule that encodes the peptide.

Provided herein, are examples of proteins, such as SlyD (SEQ ID NO: 607), that include segments of at least 4 histidine residues that make up at least 25% of the segment. The peptides identified by this embodiment can include a carboxy or amino-terminal portion, which includes a segment that includes at least 4 histidine residues and 25% histidine.

In another embodiment, the present invention provides an isolated peptide identified using the method discussed above, or a fusion protein that includes an isolated peptide identified using the method discussed above. Peptides of this embodiment that were identified from SlyD are illustrated in Example 4. These peptides include SlyDC1 (amino acids 149-196) (SEQ ID NO: 608) (see SlyD sequence below), SlyDC2 (amino acids 149-165) (SEQ ID NO: 609), SlyDC3 (amino acids 151-160) (SEQ ID NO: 610), SlyDC4 (amino acids 151-160, H159G) (SEQ ID NO: 611), SlyDC5 (amino acids 151-157) (SEQ ID NO: 612), SlyDC6 (amino acids 156-159, H159G) (SEQ ID NO: 613), and SlyDC7 (amino acids 153-159, H159G) (SEQ ID NO: 614). In other embodiments, the present invention provides a method for separating a polypeptide from a mixture that includes the polypeptide and other polypeptides, by contacting the polypeptide with a resin containing immobilized metal ions under conditions sufficient to cause the polypeptide to bind to the resin, and selectively eluting the polypeptide from the resin, wherein the polypeptide includes a peptide identified using the method discussed above.

E. coli SlyD is a 21 KDa FKBP family rotamase which catalyzes cis-trans isomerization of proline residues in order to stimulate proper folding of polypeptides (see Hottenrott et al. J Biol. Chem. 20; 272(25):15697-701, 1997). SlyD is the host protein required for lysis of E. coli upon infection with bacteriophage FX174 and has recently been shown to display rotamase (peptidylproline cis-trans-isomerase) activity. The covalent incorporation of ATP analogues into SlyD was promoted by bivalent transition metal ions (Zn²⁺ Ni²⁺>Co²⁺>Cu²⁺) but not by Mg²⁺ or Ca²⁺” (see Mitterauer et al., Biochem. J. 342:33-39, (1999)). In this regard, it can be categorized as a chaperone-like protein, and in fact is induced during stressful times in the cell, including conditions of osmotic shock, growth in cold temperatures, and long-term stationary phase growth. SlyD is unique among this family of rotamases in that its C-terminal domain contains a significant number of histidine residues (14 in 32 amino acids). This histidine-rich domain is not found in other rotamases, and its deletion seems to have no effect on the cis-prolyl isomerase activity of SlyD. The amino acid sequence of SlyD can be found under accession number P30856 in the NCBI protein database available at www.ncbi.nlm.nih.gov and is provided in Table 17.

TABLE 17
amino acid sequence of SlyD (SEQ ID NO: 607)

| | |
|---|---|
| 1 | mkvakdlvvs layqvrtedg vlvdespvsa pldylhghgs lisgietale ghevgdkfdv |
| 61 | avgandaygq ydenlvqrvp kdvfmgvdel qvgmrflaet dqgpvpveit aveddhvvvd |
| 121 | gnhmlagqnl kfnvevvair eateeelahg hvhgahdhhh dhdhdgccgg hghdhghehg |
| 181 | gegccggkgn ggcgch |

SlyD was also independently isolated as WHP (wonderous histidine-rich protein) when it was discovered to bind tightly to nickel ions immobilized on NTA resin. At least one group has accidentally purified SlyD when attempting to express and purify an unrelated His-tagged rotamase, and numerous groups have reported contamination of Ni-NTA eluates with SlyD.

The C-terminal domain of SlyD and/or fragments thereof may be used as an alternative tag to a His6 tag for purifying fusion proteins using IMAC. Portions of the C-terminal domain may be sequentially deleted and/or mutated to identify one or more peptides for metal ion binding, and those sequences may be fused to proteins of interest.

Other potential proteins with significant His content and clustering may also be useful for this purpose. Most notably, the HypB family of proteins, which shows some similarity to the C-terminal domain of SlyD, contains several members that bind Ni ions quite well. The E. coli HypB protein does not contain a His rich domain, but HypB proteins from Bradyrhizobium and Rhizobium species have been shown to bind Ni with high affinity. The amino acid sequence of Bradyrhizobium japonicum USDA 110 HypB protein (SEQ ID NO: 615) can be found under accession number BAC52196 in the NCBI protein database available at www.ncbi.nlm.nih.gov and is provided in Table 18.

TABLE 18

Amino acid sequence of Bradyrhizobium laponicum USDA 110
HypB protein (SEQ ID NO: 615).

| | |
|---|---|
| 1 | mctvcgcsdg kasiehahdh hhdhghdhdh ghdghhhhhh ghdqdhhhhh dhahgdagll |
| 61 | dcganpagqk iagmssdrii qverdilgkn drlaadnrar fradevlafn lvsspgagkt |
| 121 | sllvravsel kdsfaigvie gdqqtsndae riratgvpai qvntgkgchl daamvgeayd |
| 181 | rlpwlnggll fienvgnlvc paafdlgeac kivvfstteg edkplkypdm faasslmlin |
| 241 | kidlasvldf dlartieyar rvnpkievlt lsartgegfa afyawirkrm aattpaamta |
| 301 | ae |

Fusion Proteins

Compositions and methods of the invention also provide/contemplate fusions comprising one or more peptides of the invention, covalently linked to an analyte (preferably a protein) of interest. The peptides so contemplated may embody one or more functionalities, such as metal binding, intein cleavage, recognition by antibodies, etc. The functionalities of the peptide(s) become properties of the analyte by virtue of the covalent linkage of the peptide(s) to the analyte. Covalent linkages may be made either at the amino- or carboxy-terminus of the analyte and/or peptide backbone, or both. Covalent linkages may also be made at one or more amino acid side chains of the analyte and/or peptide. Covalent linkages may be effected either in vitro or in vivo, using chemical or biologic means, or both.

Peptides of the invention may serve any number of purposes and a number of peptides may be added to impart one or more different functions to the fusion protein of the invention. For example, peptides may (1) contribute to protein-protein interactions both internally within a protein and with other protein molecules, (2) make the fusion protein amenable to particular purification methods, (3) enable one to identify whether the fusion protein is present in a composition (e.g., the peptide may be detectable); or (4) give the fusion protein other functional characteristics.

Fusion proteins may contain one or more peptides of the invention. Typically, fusion proteins that contain more than one peptide of the invention will contain these peptides at one terminus or both termini (i.e., the N-terminus and the C-terminus) of the fusion protein, although one or more peptides may be located internally instead of, or in addition to, those present at termini. Further, more than one peptide may be present at one terminus, internally and/or at both termini of the fusion protein. For example, three consecutive peptides could be linked end-to-end at the N-terminus of fusion proteins of the invention. The invention further includes compositions and reaction mixtures which contain the above fusion proteins, as well as methods for preparing these fusion proteins, nucleic acid molecules (e.g., vectors) which encode these fusion proteins and recombinant host cells which contain these nucleic acid molecules.

In some embodiments, it may be desirable to remove all or a portion of a peptide of the invention from a fusion protein comprising the peptide and an additional protein sequence (e.g., an enzyme). In embodiments of this type, one or more amino acids forming one or more protease cleavage sites, e.g., for a protease enzyme, may be incorporated into the primary sequence of the fusion protein. A protease site may be located such that cleavage at the site may remove all or a portion of the peptide sequence from the fusion protein.

In some embodiments, the protease site may be located between the peptide sequence and the additional protein sequence such that all of the peptide sequence is removed by cleavage with a protease enzyme that recognizes the protease site. In some instances, it is preferred that the amino acid sequence for cleavage is positioned at the N-terminal side of a polypeptide of interest, so that enzymatic cleavage results in the production of the polypeptide with a desired N-terminal sequence, which may be the N-terminal sequence of the protein as it occurs naturally with or without additional amino acids (for an overview, see Jonasson et al., Biotechnol. Appl. Biochem. 35:91-105, 2002).

Any appropriate protease cleavage site can be incorporated into the proteins and vectors of the present invention. Typically, the protease cleavage site may be greater than 4 amino acid residues. Examples of suitable protease sites include, but are not limited to, the Factor Xa cleavage site having the sequence Ile-Glu-Gly-Arg (SEQ ID NO: 616), which is recognized and cleaved by blood coagulation factor Xa, and the thrombin cleavage site having the sequence Leu-Val-Pro-Arg (SEQ ID NO: 617), which is recognized and cleaved by thrombin.

Another suitable protease site is one recognized by the tobacco etch virus (TEV) protease, e.g., TEV NIa protease. The TEV protease cleaves a specific consensus cleavage site which spans the seven amino acid sequence E-X-V/I/L-Y-X-Q*S/G (SEQ ID NO: 618), wherein X can be any amino acid residue (Doughtery et al., EMBO J., 7:1281-1287, 1988). An exemplary TEV cleavage site is E-N-L-Y-F-Q*G (SEQ ID NO: 619) (Parks et al., Anal. Biochem. 216:413-417 (1994). Patents and Published Applications regarding the TEV protease and uses thereof include U.S. Pat. No. 5,179,007; U.S. Pat. No. 5,532,142; EU 0 682 709 A1, A2, A3; WO 94/183331 A2, A3; WO 00/00625; and WO 01/96539 A2, A3.

Another suitable class of protease sites are those recognized by caspases. Caspases are a family of cysteine proteases that are key mediators in the signaling pathways for apoptosis and cell disassembly (Thomberry, Chem. Biol. 5:R97-R103, 1998). Caspase-mediated cleavage is specified by three or more amino acids immediately preceding an aspartate residue (Garcia-Calvo, M. et al., Cell Death Differen. 6:362, 1999; Thomberry et al., J. Biol. Chem. 272:17907-17911, 1997; Talanian, R. V. et al. J. Biol. Chem. 272:9677, 1997). Additional constraints are placed on the specificity; although many cellular proteins have the correct amino acid sequence required for caspase cleavage, only a select group of proteins are hydrolyzed.

The caspases have been classified into three groups depending on the amino acid sequence that is preferred or primarily recognized. The group of caspases that includes caspases 1, 4, and 5 has been shown to prefer hydrophobic aromatic amino acids at position 4 on the N-terminal side of the cleavage site. Another group, which includes caspases 2, 3 and 7, recognizes aspartyl residues at both positions 1 and 4 on the N-terminal side of the cleavage site, and preferably a sequence of Asp-Glu-Xaa-Asp (SEQ ID NO: 620). A third group, which includes caspases 6, 8, 9 and 10, tolerates many amino acids in the primary recognition sequence, but seems to prefer residues with branched, aliphatic side chains such as valine and leucine at position 4.

Another suitable protease site is that recognized V8 protease. Natural V8 protease is a serine protease secreted by Staphylococcus aureus V8 in culture medium. It specifically cleaves a C-terminal peptide bond between glutamic acid and aspartic acid (Houmard et al., Proc. Natl. Acad. Sci. U.S.A. 69:3506-3509, 1971). A DNA nucleotide sequence coding for the amino acid sequence of the natural V8 protease has been described (Carmona et al., Nucleic Acid Res. 15:6757, 1987). Mutant V8 proteases have also been described (see, e.g., U.S. Pat. No. 5,747,321 to Yabuta et al., entitled “Mutant Staphylococcus aureus V8 proteases”).

Additional protease sites include, but are not limited to, the cleavage sites for enterokinase, trypsin, chymotrypsin, Genenase I, and Furin. Genenase™I (Genencor International, Inc.) cleaves after the tyrosine in the sequences HY and YH. Thus, a peptide of the invention comprising the sequence MHYYHY (SEQ ID NO: 575), theoretically provides three substrate sites for Genenase™I. Other suitable protease sites are known to those skilled in the art and may be used in conjunction with the present invention.

Fusion proteins of the invention may be produced using any technique known to those skilled in the art. For example, a fusion protein may be translated from a nucleic acid molecule encoding one or more peptide of the invention and one or more additional sequences coding for one or more additional polypeptides. One example of a fusion protein is a protein translated from a mRNA that is transcribed from a DNA molecule that encodes an affinity peptide of the invention fused in frame to a cDNA which contains all or part of a naturally occurring open reading frame. In this example, as well as in other examples, the DNA transcribed may be obtained from any source.

A fusion protein may comprise a plurality of contiguous amino acid sequences that are not naturally found in the same protein. For example, a fusion protein of the invention may comprise a peptide of the invention and may further contain one or more sequences imparting a desired activity or characteristic to the fusion protein. For example, a fusion protein may comprise a secretion sequence or secretion signal sequence (i.e., an amino acid signal sequence that leads to the transport of a protein containing the signal sequence outside the cell membrane). In the present case, a fusion protein of the present invention may contain such a secretion sequence to enhance and simplify purification. Representative examples of secretion signal sequences are well known to those having ordinary skill in this art.

The invention provides for peptides and/or fusion proteins, which bind to IMAC matrices and may, optionally, have one or more additional useful properties. Peptide and/or fusion proteins may have tissue-specific localization properties (see, for example, Pasqualini R, Ruoslahti E., 1996, Nature 380(6572):364-6) such as kidney, brain, bone, lung, and the like, and tumor tissue present in these, and other tissues. Peptides and/or fusion proteins of the invention may comprise cellular targeting elements. Cellular targeting elements may direct fusion proteins of the invention to specific cell types and include, but are not limited to, antibody fragments directed to a cellular surface molecule, fragments of ligands for receptors present on a cell, cell-specific targeting sequences derived from pathogens, derivatives of cellular adhesion molecules, and the like. Peptides and/or fusion proteins of the invention may comprise intacellular targeting elements. Intracellular targeting elements may direct fusion proteins to subcellular locations including, without limitation, the nucleus, the cell membrane, the chloroplast, the mitochondrion, the endoplasmic reticulum, the cytoplasm, and membranes or intermembrane spaces of any of the preceding, are known and are commercially available (e.g., Invitrogen's line of pShooter™ vectors). A nucleotide sequence that localizes nucleic acids to mitochondria is described in U.S. Pat. No. 5,569,754.

Peptides and/or fusion proteins of the invention also may comprise signals or sequences for translocation into and/or between cells by enabling transit across a cell membrane and/or wall.

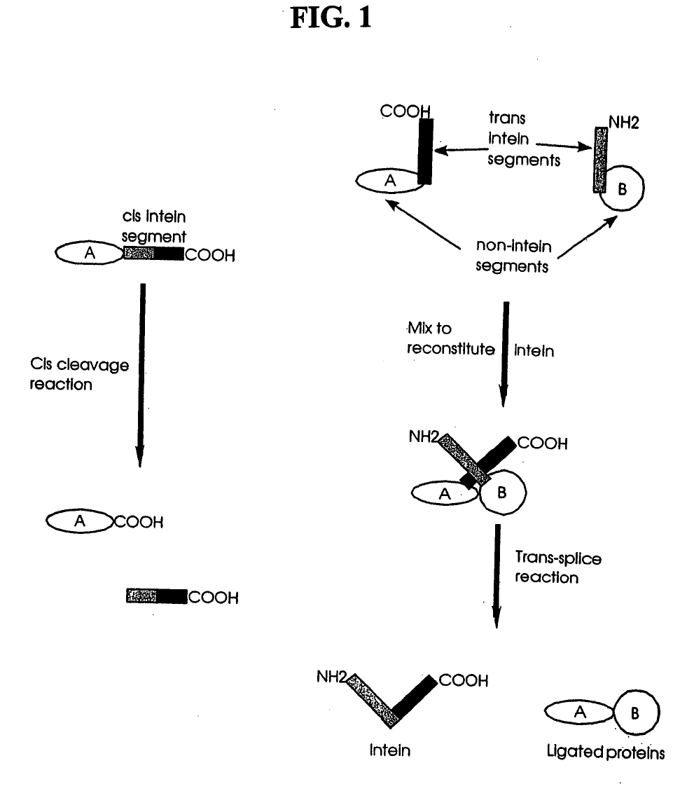

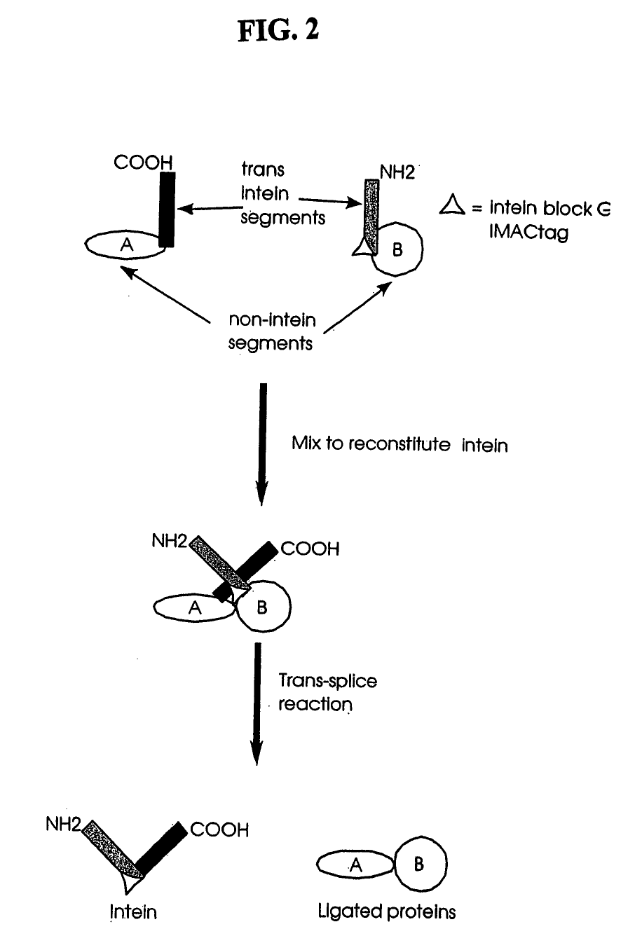

Peptides of the invention and/or fusion proteins comprising such peptides may comprise a plurality of functional characteristics. An example of such multifunctional peptides and/or fusion proteins are those that comprise intein splice sites, in addition to IMAC utility. Inteins can function in cis or trans (see FIG. 1). The sequence EHGMGHNT (SEQ ID NO: 606) reasonably meets the criteria for eubacterial intein splicing block G (see Perler, 2000, Nucleic Acids Res. 28(1):344-5, and http://www.neb.com/inteins/intein_intro. html). Thus, a peptide having the sequence EHGMGHNT (SEQ ID NO: 606) might serve as an IMAC tag that could then facilitate its own removal from the (fusion) protein of interest via an intein-mediated reaction. The resultant column eluate would then comprise only “native” (non-adducted) protein of interest (see FIG. 2).

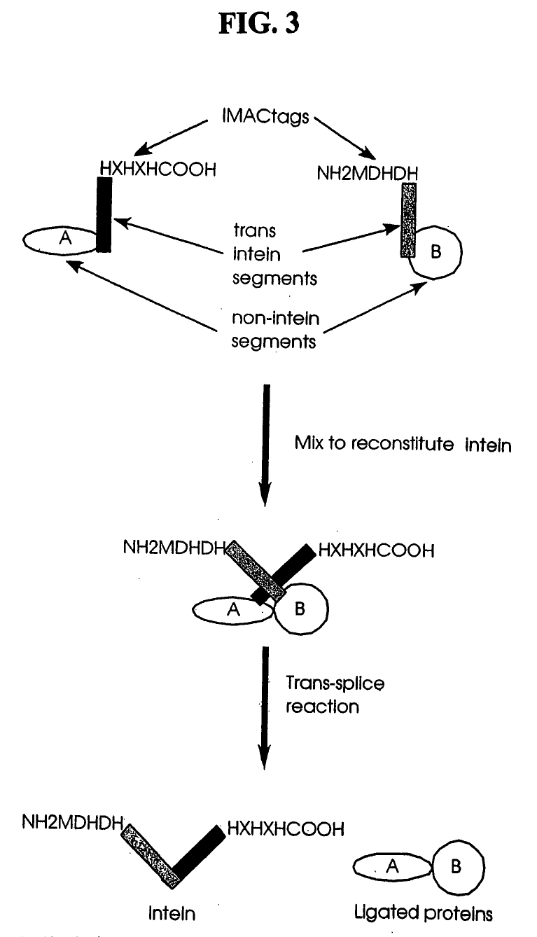

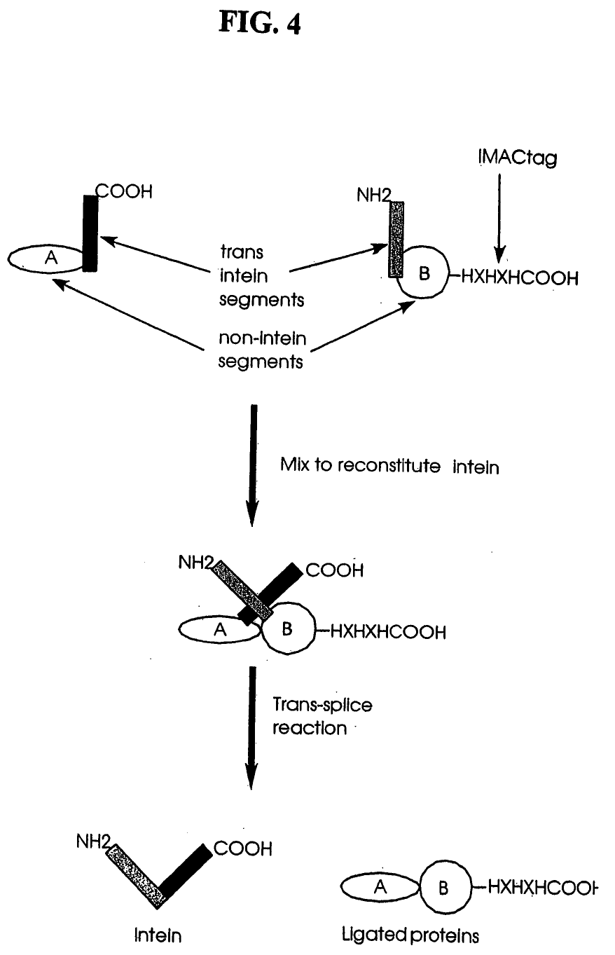

In a related embodiment, peptides of the invention may also embody motifs comprising intein splice sites that are designed to function in trans (see, for example, Martin, et al., 2001, Biochemistry 40(5): 1393-402; and Evans, et al., 2000, J Biol Chem 275(13):9091-4) Trans-splicing allows for in vitro post-translational modification of a protein or proteins of interest by intein-catalyzed ligation. Portions of the starting molecules embodying intein functionality are removed as a function of the intein reaction (see FIG. 1). Thus, additional functionalities that may have been resident in the intein moieties may be effectively removed by the trans-splicing reaction (see FIG. 3). In a related embodiment, functionalities resident in a given protein segment may be added to by exploiting the intein trans-splicing reaction (see FIG. 4). Thus, the present invention provides compositions wherein an IMAC moiety is operably linked to a protein of interest via an intein moiety.

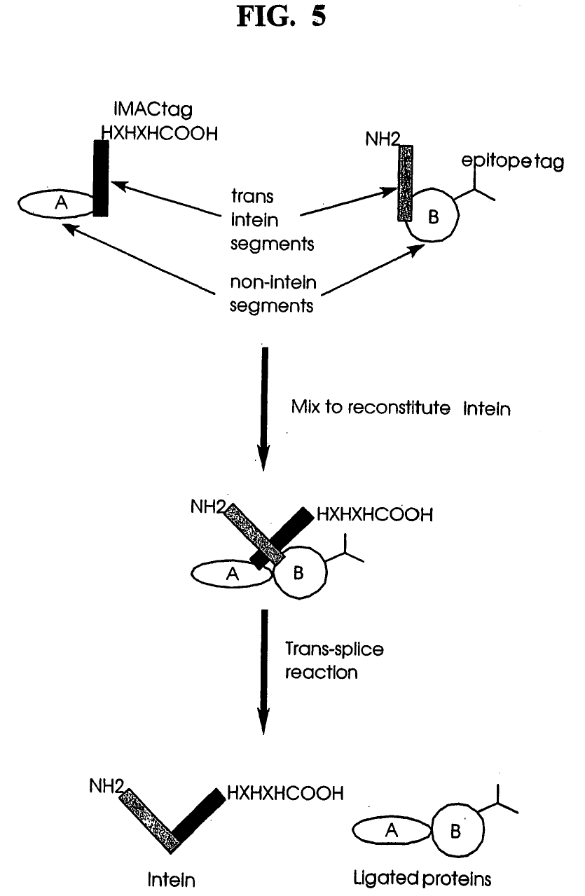

In a related aspect, the invention provides compositions of intein moieties that also comprise IMAC functionalities. Segments (meaning contiguous, but not necessarily continuous, regions of amino acids within the peptide and/or fusion protein of the invention) that embody these properties may or may not be positionally superimposed with respect to the primary sequence of the peptide or protein. For example, a segment that embodies IMAC functionality may positionally coincide with a segment that either partially or completely, embodies intein functionality (see FIG. 2). Alternatively, segments embodying IMAC and intein functionalities may be positionally disparate (see FIGS. 4-5).

In a related aspect, IMAC function and trans-splicing can be differentially enabled (see, for example, Ghosh, et al., 2001, J Biol Chem 276(26): 24051-8). Two important aspects of protein splicing were investigated by employing the trans-splicing intein from the dnaE gene of Synechocystis sp. PCC6803. First, it was demonstrated that both protein splicing and cleavage at the N-terminal splice junction were inhibited in the presence of zinc ion. The trans-splicing reaction was partially blocked at a concentration of 1-10 μM Zn²⁺ and completely inhibited at 100 μM Zn²⁺. The inhibition by zinc was reversed in the presence of ethylenediaminetetraacetic acid. Thus, the present invention includes the use of metal ions, (e.g., Zn²⁺, Ni²⁺, Co²⁺, Cu²⁺, etc) in conjunction with the peptides and/or fusion proteins of the invention to modulate intein reactions involving such peptides and/or fusion proteins.

In another aspect, peptides of the invention also embody protein labeling technology. For instance, a peptide such as R1-HGGEGGH-R2 (SEQ ID NO: 621), where R1 represents a covalent or non-covalent linking moiety, and R2 represents a detectable label, may be exploited to generically label proteins of interest. The labeled protein may then be partitioned away from a complex mixture of proteins by affinity purification using an IMAC matrix. In a related aspect, intein chemistry can be exploited to effect protein derivatization and/or labeling. (see FIG. 5).

In another aspect, IMAC peptides of the invention also embody epitopes. In related embodiments, antigenic or epitopic peptides, or segments thereof, may comprise one or more of the functions described above.

Fusion proteins of the invention may comprise peptides of the invention fused to an additional polypeptide sequence. Any polypeptide sequence known to those skilled in the art may be used in conjunction with the peptides disclosed herein to prepare a fusion protein of the invention.

Examples of suitable polypeptide sequences that may be used in conjunction with the peptides of the invention to produce fusion proteins of the invention, include, but are not limited to
enzymes, e.g., kinases; peptidases/proteinases; oxidoreductases; nucleases; recombinases (including Cre, Int, Flp, Tn5 resolvase, and the like); ligases (including DNA ligases and the like); lyases; isomerases (including toposiomerases and the like); polymerases (including DNA polymerases, RNA polymerases, reverse transcriptases, and the like); transferases (including terminal transferases, glutathione S-transferases, and the like); ATPases; GTPases; etc.;
cytokines, e.g., growth factors (such as epidermal growth factor (EGF), fibroblast growth factors (FGFs), keratinocyte growth factors (KGFs), hepatocyte growth factors (HGFs), platelet-derived growth factor (PDGF), transforming growth factors alpha and beta (TGF-α and TGF-β), neurotrophic factor (NTF), ciliary neurotrophic factor (CNTF), brain-derived neurotrophic factor (BDNTF), glial-derived neurotrophic factor (GDNTF), bone morphogenic proteins (BMPs), and the like, and variants thereof); interleukins (such as IL-1 through IL-18, and the like, and variants thereof); interferons (such as IFN-α, IFN-β, IFN-γ, and the like, and variants thereof); colony-stimulating factors (such as granulocyte colony-stimulating factor (G-CSF), macrophage colony-stimulating factor (M-CSF), granulocyte-macrophage colony-stimulating factor (GM-CSF); erythropoietin (Epo); thrombopoietin (Tpo); leukemia inhibitory factor (LIF/Steel Factor); tumor-necrosis factors (TNFs); and the like, and variants thereof); peptide hormones (such as antidiuretic hormone, chorionic gonadotropin, leutenizing hormone, follicle-stimulating hormone, insulin, prolactin, somatomedins, growth hormone, thyroid-stimulating hormone, placental lactogen, and the like, and variants thereof); etc.;
intraceullar signaling peptides;
receptors (e.g., cytokine receptors, hormone receptors, antibody receptors, integrins and other extracellular matrix receptors, neurotransmitter receptors, viral receptors, and the like, and variants thereof);
antibodies (e.g., polyclonal or monoclonal antibodies, fragments thereof (including Fab and Fc fragments and portions thereof), and multi-antibody complexes);
vaccine components (including, but not limited to, proteins or peptides of etiologic agents such as viruses, bacteria, fingi (including yeasts), parasites and the like; proteins or peptides of tumor cells or other cancer-related proteins or peptides; and other proteins or peptides against which it is desirable to produce an immune response in an animal, suitably a mammal such as a human);
structural and/or functional proteins or peptides (e.g., hemoglobin, albumins including serum albumins, cytoskeletal proteins, transmembrane channel proteins or peptides, and the like, and fragments or variants thereof);
synthetic peptides (e.g., polylysine, and other synthetic peptides of any length containing a desired sequence of two or more amino acids linked together by peptide bonds to form a peptide, oligopeptide, polypeptide or protein, any and all of which can be produced by art-known methods of synthetic peptide synthesis that will be familiar to the ordinarily skilled artisan, and that are described herein); and the like.

Other suitable peptides, oligopeptides, polypeptides and proteins suitable for use in accordance with the present invention (i.e., in the fusion proteins of the invention) will be familiar to one of ordinary skill and therefore are encompassed by the present invention.

Peptides and/or fusion proteins of the invention may comprise any number and combination of the characteristics and/or elements described above.

Nucleic Acid Molecules

The present invention encompasses nucleic acid molecules that encode peptides of the invention as well as compositions comprising such nucleic acid molecules. Nucleic acid molecules may be DNA, RNA, or combinations thereof. A particular nucleic acid of the invention may encode one or more peptides of the invention. In a related embodiment, a nucleic acid of the invention may encode the peptide and/or an analyte molecule (e.g. a protein). The nucleic acid segments that encode the peptide and analyte may be contiguous, such that in the transcription and/or translation products of the coding segments, the segments are juxtaposed. In some embodiments, the coding sequences of the peptide of the invention and a protein may be separated by one or more sequences that are non-coding. Thus, the present invention encompasses nucleic acid molecules containing one or more intervening sequences (introns) that may be transcribed from a DNA molecule into an RNA molecule and subsequently removed (e.g., by splicing) prior to translation of the RNA molecule into protein. Nucleic acid molecules of the invention may be synthesized in vitro, in vivo, or by the action of cell-free transcription.

Vectors

In certain embodiments, the nucleic acid molecules of the invention are provided as vectors, particularly cloning vectors, expression vectors or gene therapy vectors. Vectors according to this aspect of the invention can be double-stranded or single-stranded and which may be DNA, RNA, or DNA/RNA hybrid molecules, in any conformation including but not limited to linear, circular, coiled, supercoiled, torsional, nicked and the like. These vectors of the invention include but are not limited to plasmid vectors and viral vectors, such as a bacteriophage, baculovirus, retrovirus, lentivirus, adenovirus, vaccinia virus, semliki forest virus and adeno-associated virus vectors, all of which are well-known and can be purchased from commercial sources (Invitrogen; Carlsbad, Calif.; Promega, Madison Wis.; Stratagene, La Jolla Calif.).

Vectors of the invention are typically biologically replicable nucleic acid molecules, and may encode peptides and peptide fusions of the invention. Biologically replicable nucleic acid molecules may comprise chromosomes, plasmids, phage, viruses, or any hybrid of the aforesaid nucleic acid molecules.

In accordance with the invention, any vector may be used to construct the fusion proteins used in the methods of the invention. In particular, vectors known in the art and those commercially available (and variants or derivatives thereof) may in accordance with the invention be engineered to include one or more recombination sites for use in the methods of the invention. Such vectors may be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, Perkin Elmer, Pharmingen, and Research Genetics. Such vectors may then for example be used for cloning or subcloning nucleic acid molecules of interest. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts and the like.

Other vectors of interest include viral origin vectors (M13 vectors, bacterial phage 8 vectors, adenovirus vectors, and retrovirus vectors), high, low and adjustable copy number vectors, vectors which have compatible replicons for use in combination in a single host (pACYC184 and pBR322) and eukaryotic episomal replication vectors (pCDM8).

The isolated DNA molecules of the invention may be inserted into standard nucleotide vectors suitable for transfection or transformation of a variety of prokaryotic (bacterial) or eukaryotic (yeast, plant or animal including human and other mammalian) host cells. Vectors suitable for these purposes, and methods for insertion of DNA fragments therein, will be well-known to one of ordinary skill in the art. Thus, the present invention also relates to vectors comprising such DNA molecules, and to host cells comprising such DNA molecules and/or vectors.

Particular vectors of interest include prokaryotic expression vectors such as pProEx-HT, pcDNA II, pSL301, pSE280, pSE380, pSE420, pTrcHisA, B, and C, pRSET A, B, and C (Invitrogen Corporation), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and variants and derivatives thereof. Vectors can also be made from eukaryotic expression vectors such as pYES2, pAC360, pBlueBacHis A, B, and C, pVL1392, pBsueBacIII, pCDM8, pcDNA1, pZeoSV, pcDNA3 pREP4, pCEP4, pEBVHis, pFastBac, pFastBac HT, pFastBac DUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and variants or derivatives thereof.

Other vectors of particular interest include pUC18, pUC19, pBlueScript, pSPORT, cosmids, phagemids, YACs (yeast artificial chromosomes), BACs (bacterial artificial chromosomes), MACs (mammalian artificial chromosomes), HACs (human artificial chromosomes), P1 (E. coli phage), pQE70, pQE60, pQE9 (Qiagen), pBS vectors, PhageScript vectors, BlueScript vectors, pNH8A, pNH16A, pNH18A, pNH46A (Stratagene), pcDNA3, pSPORT1, pSPORT2, pCMVSPORT2.0 and pSV-SPORT1 (Invitrogen), pGEX, pTrsfus, pTrc99A, pET-5, pET-9, pKK223-3, pKK233-3, pDR540, pRIT5 (Pharmacia), and variants or derivatives thereof.

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZa, pGAPZ, pGAPZa, pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; 8ExCell, 8 gt11, pTrc99A, pKK223-3, pGEX-18T, pGEX-2T, pGEX-2TK, pGEX-4T-1, pGEX-4T-2, pGEX-4T-3, pGEX-3×, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32 LIC, pET-30 LIC, pBAC-2 cp LIC, pBACgus-2 cp LIC, pT7Blue-2 LIC, pT7Blue-2, 8SCREEN-1, 8BIueSTAR, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, p∃gal-Basic, p∃gal-Control, p∃gal-Promoter, p∃gal-Enhancer, pCMV∃, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX 4T-1/2/3, pYEX-S1, pBacPAK-His, pBacPAK8/9, pAcUW31, BacPAK6, pTrip1Ex, 8gt10, 8gt11, pWE15, and 8Trip1Ex from Clontech; Lambda ZAP II, pBK-CMV, pBK-RSV, pBluescript II KS +/−, pBluescript II SK +/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS +/−, pBC KS +/−, pBC SK +/−, Phagescript, pCAL-n-EK, pCAL-n, pCAL-c, pCAL-kc, pET-3abcd, pET-1 labcd, pSPUTK, pESP-1, pCMVLacI, pOPRSVI/MCS, pOPI3 CAT, pXT1, pSG5, pPbac, pMbac, pMC1neo, pMC1neo Poly A, pOG44, pOG45, pFRT∃GAL, pNEO∃GAL, pRS403, pRS404, pRS405, pRS406, pRS413, pRS414, pRS415, and pRS416 from Stratagene.

Two-hybrid and reverse two-hybrid vectors of particular interest include pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

Vectors of the invention may be compatible with any cloning technique known to those skilled in the art (e.g., recombinational cloning, topoisomerase-mediated cloning etc.). In some embodiments, a vector for use in the present invention may be a vector comprising one or more recombination sites, such as those disclosed in U.S. Pat. Nos. 5,888,732, 6,143,557, 6,171,861, 6,270,969, and 6,277,608. Vectors comprising one or more recombination sites are commercially available, for example, from Invitrogen Corporation, Carlsbad, Calif. and may be used in recombinational cloning techniques such as those describe in the GATEWAY™ Cloning Technology product literature also available from Invitrogen Corporation, Carlsbad, Calif. Examples of suitable vectors comprising one or more recombination site include, but are not limited to, pDEST R4-R3, pDEST10, pDEST14, pDEST1, pcDNA3.1/nV5-DEST, pcDNA3.2-DEST, pcDNA3.2/GW/D-TOPO, pcDNA6.2-DEST, pDONR201, pDONR20, pDONR22, and pEXP2-DEST all commercially available from Invitrogen Corporation, Carlsbad, Calif.

In some embodiments, vectors of the invention may be compatible with topisomerase-mediated cloning techniques such as those disclosed in U.S. Pat. Nos. 6,548,277 and 5,766,891. Vectors comprising one or more sequences recognized by a topoisomerase enzyme and useful in topisomerase-mediated cloning are commercially available from, for example, Invitrogen Corporation, Carlsbad, Calif. Examples of suitable vectors for topoisomerase-mediated cloning include, but are not limited to, pBAD/Thio-TOPO, pBAD/TOPO, pBAD102/D-TOPO, pBAD202/D-TOPO, pBlue-TOPO, pBlueBac4.5/V5-His-TOPO, pcDNA3.1/CT-GFP-TOPO, pcDNA3.1/NT-GFP-TOPO, pcDNA3.2/GW/D-TOPO, pCR-Blunt II-TOPO, pCR-XL-TOPO, pCRT7/CT-TOPO, pEF5/FRT/V5-D-TOPO, pENTR/D-TOPO, pENTR/SD/D-TOPO, pGlow-TOPO, and pLenti6N5-D-TOPO, all of which are commercially available from Invitrogen Corporation, Carlsbad, Calif.

Other cloning vectors include plasmids, cosmids, viral or phage DNA molecules or other DNA molecules that are capable of autonomous replication in a host cell, via splicing of vector-borne nucleic acid into the genetic material (chromosomal or extrachromosomal) of the host cell without loss of an essential biological function of the vector, thereby facilitating the replication and cloning of the vector. The cloning vector may further contain a marker suitable for use in the identification of cells transformed with the cloning vector. Markers may be, for example, antibiotic resistance genes, e.g., tetracycline resistance or ampicillin resistance. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Expression vectors according to the invention include vectors that are capable of enhancing the expression of one or more genes that have been inserted or cloned into the vector, upon transformation of the vector into a host. The cloned gene is usually placed under the control of (i.e., operably linked to) certain transcriptional regulatory sequences such as promoter sequences. In certain preferred embodiments in this regard, the vectors provide for specific expression, which may be inducible and/or cell type-specific. Particularly preferred among such vectors are those inducible by environmental factors that are easy to manipulate, such as temperature and nutrient additives. Expression vectors useful in the present invention include chromosomal-, episomal- and virus-derived vectors, e.g., vectors derived from bacterial plasmids or bacteriophages, and vectors derived from combinations thereof, such as cosmids and phagemids.

To produce expression vectors according to this aspect of the invention, one or more gene-containing nucleic acid molecules or oligonucleotide inserts should be operatively linked to an appropriate promoter in the vector (which may be provided by the vector itself (i.e., a “homologous promoter”) or may be exogenous to the vector (i.e., a “heterologous promoter), such as the phage lambda P_{L} promoter, the E. coli lac, trp and tac promoters, and the like. Other suitable promoters will be known to the skilled artisan. The gene fusion constructs will further contain sites for transcription initiation, termination and, in the transcribed region, a ribosome binding site for translation. The coding portion of the mature transcripts expressed by the constructs will preferably include a translation initiation codon at the beginning, and a termination codon (UAA, UGA or UAG) appropriately positioned at the end, of the polynucleotide to be translated. The expression vectors also preferably include at least one selectable marker. Such markers include tetracycline or ampicillin resistance genes for culturing in E. coli and other bacteria.

Viral expression vectors can be particularly useful where a method of the invention is practiced for the purpose of generating a ds recombinant nucleic acid molecule covalently linked in one or both strands, that is to be introduced into a cell, particularly a cell in a subject. Viral vectors provide the advantage that they can infect host cells with relatively high efficiency and can infect specific cell types or can be modified to infect particular cells in a host.

Viral vectors have been developed for use in particular host systems and include, for example, bacteriophage vectors (e.g., phage lambda), which infect bacterial cells (for review, see Baneyx F., Curr Opin. Biotechnol. 10:411-421 (1999)), baculovirus vectors, which infect insect cells; retroviral vectors, other lentivirus vectors such as those based on the human immunodeficiency virus (HIV), adenovirus vectors, adeno-associated virus (AAV) vectors, herpesvirus vectors, vaccinia virus vectors, and the like, which infect mammalian cells (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference). For example, a viral vector based on an HIV can be used to infect T cells, a viral vector based on an adenovirus can be used, for example, to infect respiratory epithelial cells, and a viral vector based on a herpesvirus can be used to infect neuronal cells. Other vectors, such as AAV vectors can have greater host cell range and, therefore, can be used to infect various cell types, although viral or non-viral vectors also can be modified with specific receptors or ligands to alter target specificity through receptor mediated events.

Host Cells

The present invention encompasses host cells comprising one or more nucleic acid molecule invention (e.g., a nucleic acid molecule encoding one or more peptide of the invention). Representative host cells that may be used with the invention include, but are not limited to, bacterial cells, yeast cells, plant cells and animal cells. Bacterial host cells suitable for use with the invention include Escherichia spp. cells (particularly E. coli cells and most particularly E. coli strains DH10B, Stb12, DH5α, DB3, DB3.1 (e.g., E. coli LIBRARY EFFICIENCY® DB3.1™ Competent Cells; Invitrogen Corp., DB4 and DB5; see U.S. application Ser. No. 09/518,188, filed on Mar. 2, 2000, the disclosure of which is incorporated by reference herein in its entirety), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Animal host cells suitable for use with the invention include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly CHO, COS, VERO, BHK and human cells). Yeast host cells suitable for use with the invention include Saccharomyces cerevisiae cells and Pichia pastoris cells. These and other suitable host cells are available commercially, for example from Invitrogen Corporation, Carlsbad, Calif., the American Type Culture Collection (Manassas, Va.); and the Agricultural Research Culture Collection (NRRL; Peoria, Ill.).

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, transfection, and transformation. The nucleic acid molecules and/or vectors of the invention may be introduced alone or in conjunction with other the nucleic acid molecules and/or vectors. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E., From Genes to Clones, New York: VCH Publishers (1987), which are illustrative of the many laboratory manuals that detail these techniques and which are incorporated by reference herein in their entireties for their relevant disclosures.

Purification

The present invention relates to the purification of molecules comprising one or more peptides invention. Such molecules may be fusion proteins comprising one or more peptides of the invention fused to one or more polypeptide sequences. Examples of molecules to be purified using the methods of the present invention include recombinant peptides and/or fusion proteins produced by transformed host cells. Such peptides and/or fusion proteins are typically produced in a soluble form and/or are secreted from the host cell. Fusion proteins of the invention to be purified using the techniques described herein may comprise one or more metal ion chelating peptide of the invention. Such fusion proteins may reversibly bind to a chromatography resin comprising immobilized metal ions (e.g., Ni²⁺, Co²⁺, Cu²⁺, and other divalent cations).

Fusion proteins may be purified from the host cell or from the host cell culture medium into which they have been secreted. Typically, when purified from a host cell, the host cell is lysed using standard techniques (e.g., enzymatic digestion, sonication, French press, etc.) to form a lysate comprising the fusion protein. A fusion protein of the invention may be purified from a lysate or from a host cell culture medium material by contacting the lysate or medium with a suitable chromatography medium (e.g. a medium comprising an immobilized metal ion) under conditions suitable for binding of the fusion protein to the chromatography medium. The lysate or culture medium may be contacted with the chromatography medium in either a batchwise technique (e.g. by mixing the chromatography medium with the lysate or culture medium) or column technique. The bound fusion protein may be washed one or more times to remove any materials that do not bind as tightly to the chromatography medium. The washed protein may then be eluted from the medium by contacting the medium with a suitable elution buffer. In the case where the immobilized metal ion is a Ni²⁺ ion, a suitable buffer may comprise imidazole, for example, at about 0.5 M.

As discussed above, a fusion protein may comprise a cleavage site for a protease, for example, located between a peptide of the invention and a protein of interest. After elution from the chromatography medium or while still bound to the medium, a fusion protein of the invention may be contacted with a solution comprising a protease enzyme that cleaves at the cleavage site.

Antibodies

The present invention concerns the production and use of molecules (polypeptides and antibodies) that are capable of “specific binding” to one another. As used herein, a molecule is said to be capable of “specific binding” to another molecule, if such binding is dependent upon the respective and specific structures of the molecules. The known capacity of an antibody to bind to an antigen is an example of “specific binding.” Such interactions are in contrast to non-specific binding between classes of compounds, irrespective of their chemical structure (such as the binding of proteins to nitrocellulose, etc.). Most preferably, the antibodies of the present invention exhibit “highly specific binding,” such that they will be incapable or substantially incapable of binding to closely related polypeptides (e.g., the peptides and/or fusion proteins of the invention). Indeed, preferred antibodies of the present invention exhibit the capacity to bind to a peptide or protein of Tables 1-18 or other peptides disclosed herein. For example, antibodies to the His6 peptide are known (see Muller et al. Anal. Biochem. 1998 May 15; 259(1):54-61).

The present invention further relates to antibodies and T-cell antigen receptors (TCR) which specifically bind the peptides and/or fusion proteins of the invention. Antibodies may be polyclonal and/or monoclonal. They may be prepared against an entire polypeptide or against a fragment of the polypeptide.

The antibodies of the present invention include IgG (including IgG1, IgG2, IgG3, and IgG4), IgA (including IgA1 and IgA2), IgD, IgE, IgM, and IgY. As used herein, the term “antibody” (Ab) is meant to include whole antibodies, including single-chain whole antibodies, and antigen-binding fragments thereof. In some embodiments, antigen-binding fragments may be mammalian antigen-binding antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain.

Antibodies of the invention may be prepared from any animal origin including birds and mammals. Preferably, the antibodies prepared from mammals, (e.g., human, murine, rabbit, goat, guinea pig, camel, or horse). Other preferred sources may be avian (e.g., chicken).

Antibodies may be used for the detection of the polypeptides in an immunoassay, such as ELISA, Western blot, radioimmunoassay, enzyme immunoassay, and may be used in immunocytochemistry. In some embodiments, an anti-polypeptide antibody may be in solution and the polypeptide to be recognized may be in solution (e.g., an immunopreciptitation) or may be on or attached to a solid surface (e.g., a Western blot). In other embodiments, the antibody may be attached to a solid surface and the polypeptide may be in solution (e.g., affinity chromatography).

Antibodies to the peptides and/or fusion proteins of the invention may be used to determine the presence, absence or amount of one or more polypeptides in a sample. The amount of specifically bound polypeptide may be determined using an antibody to which is attached a label or other marker, such as a radioactive, a fluorescent, or an enzymatic label. Alternatively, a labeled secondary antibody (e.g., an antibody that recognizes the antibody that is specific to the polypeptide) may be used to detect a polypeptide-antibody complex between the specific antibody and the polypeptide.

Antibodies of the invention may be used to modulate one or more activities of the peptides and/or fusion proteins of the invention. For example, one or more peptides and/or fusion proteins of the invention may be contacted with an antibody under conditions such that the antibody binds to the peptide and/or fusion protein. A peptide and/or fusion protein bound by antibody may have the same or different activities as the same peptide and/or fusion protein unbound. In some embodiments, a peptide and/or fusion protein of the invention bound by an antibody of the invention may have a reduced, substantially reduced or eliminated enzymatic activity while bound. For example, a fusion protein of the invention comprising a peptide of the invention fused to a polymerase enzyme may display no detectable RNA-dependent and/or DNA-dependent DNA polymerase activity. Preferably, the activity is recovered when the antibody is no longer bound. In some embodiments, antibodies of the present invention may bind to a polypeptide of the invention under some conditions (e.g., temperature, ionic strength, etc.) and may not bind under other conditions (e.g., at an elevated temperature).

One or more of the peptides and/or fusion proteins of the invention may be used as immunogens to prepare polyclonal an/or monoclonal antibodies capable of binding the peptides and/or fusion proteins using techniques well known in the art (Harlow & Lane, Antibodies: A Laboratory Manual, Cold Spring Harbor Laboratory, Cold Spring Harbor, N.Y., 1988). In brief, antibodies are prepared by immunization of suitable subjects (e.g., mice, rats, rabbits, goats, etc.) with all or a part of the peptide and/or fusion protein of the invention. If the peptide and/or fusion protein or fragment thereof is sufficiently immunogenic, it may be used to immunize the subject. If necessary or desired to increase immunogenicity, the peptide and/or fusion protein or fragment may be conjugated to a suitable carrier molecule (e.g., BSA, KLH, and the like). Peptides and/or fusion proteins of the invention or fragments thereof may be conjugated to carriers using techniques well known in the art. For example, they may be directly conjugated to a carrier using, for example, carbodiimide reagents. Other suitable linking reagents are commercially available from, for example, Pierce Chemical Co., Rockford, Ill.

Suitably prepared peptides and/or fusion proteins of the invention or fragments thereof may be administered by injection over a suitable time period. They may be administered with or without the use of an adjuvant (e.g., Freunds). They may be administered one or more times until antibody titers reach a desired level.

In some embodiments, it may be desirable to produce monoclonal antibodies to the peptides and/or fusion proteins of the invention or fragments thereof. Monoclonal antibodies can be prepared from the immune cells of animals (e.g., mice, rats, etc.) immunized with all or a portion of one or more peptides and/or fusion proteins of the invention using conventional procedures, such as those described by Kohler and Milstein, Nature, 256, pp. 495-497 (1975). Hybridoma cell lines may be prepared by isolating antibody secreting cells of the host animal from lymphoid tissue (such as the spleen) and fusing them with mouse myeloma cells (for example, SP2/0-Ag14 murine myeloma cells) in the presence of polyethylene glycol. The fused cells may be diluted into selective media and plated in multiwell tissue culture dishes. The hybridoma cells which secrete the desired antibodies can then be identified testing the supernatants for antibodies of the desired specificity using standard techniques (e.g., ELISA, etc.). The resultant hybridoma cells can be grown in static culture, hollow fiber bioreactors or used to produce ascitic tumors in mice in order to produce the monoclonal antibodies. Thus, the present invention provides monoclonal antibodies specific to the peptides and/or fusion proteins of the invention, as well as cell lines producing such monoclonal antibodies.

In some embodiments, it may be desirable to use a fragment of an antibody that is capable of binding a peptide and/or fusion protein of the invention or fragment thereof. For example, Fab, Fab′, of F(ab′)₂ fragments may be produced using techniques well known in the art.

In some embodiments, the present invention contemplates a composition comprising a peptide and/or fusion protein of the invention and an antibody to the peptide and/or fusion protein of the invention. In such a composition, the antibody may be bound to the peptide and/or fusion protein under one set of conditions (e.g., temperature, ionic strength, etc.) and may dissociate from the polypeptide under other conditions (e.g., at an increased temperature).

Kits

The invention also relates to kits for use of nucleic acid molecules, peptides, and/or fusion proteins of the invention. Kits according to the present invention may comprise a carrying means being compartmentalized to receive in close confinement therein one or more containers such as vials, tubes, bottles, ampoules and the like. Each of such containers may comprise components or a mixture of components needed to perform recombinational cloning of nucleic acid molecules, particularly according to the methods of the present invention.

In another aspect, the invention provides kits that may be used in conjunction with methods the invention. Kits according to this aspect of the invention may comprise one or more containers, which may contain one or more components selected from the group consisting of one or more nucleic acid molecules (e.g., one or more nucleic acid molecules encoding one or more affinity peptides of the invention), one or more primers, the molecules and/or compounds of the invention, one or more polymerases, one or more reverse transcriptases, one or more recombination proteins (or other enzymes for carrying out the methods of the invention), one or more buffers, one or more detergents, one or more restriction endonucleases, one or more nucleotides, one or more terminating agents (e.g., ddNTPs), one or more transfection reagents, pyrophosphatase, and the like.

A wide variety of nucleic acid molecules can be used with the invention. Typically a nucleic acid molecule invention may encode one or more affinity peptides of the invention. In addition, nucleic acid molecules of the invention may contain promoters, sequences encoding signal peptides, enhancers, repressors, selection markers, transcription signals, translation signals, primer hybridization sites (e.g., for sequencing or PCR), recombination sites, restriction sites and polylinkers, sites that suppress the termination of translation in the presence of a suppressor tRNA, suppressor tRNA coding sequences, sequences that encode domains and/or regions for the preparation of fusion proteins, origins of replication, telomeres, centromeres, and the like. Similarly, libraries can be supplied in kits of the invention. These libraries may be in the form of replicable nucleic acid molecules or they may comprise nucleic acid molecules that are not associated with an origin of replication. As one skilled in the art would recognize, the nucleic acid molecules of libraries, as well as other nucleic acid molecules that are not associated with an origin of replication, either could be inserted into other nucleic acid molecules that have an origin of replication or would be an expendable kit components.

Further, in some embodiments, libraries supplied in kits of the invention may comprise two components: (1) the nucleic acid molecules of these libraries and (2) 5′ and/or 3′ recombination sites. In some embodiments, when the nucleic acid molecules of a library are supplied with 5′ and/or 3′ recombination sites, it will be possible to insert these molecules into nucleic acid molecules encoding one or more peptides and/or fusion proteins of the invention, which also may be supplied as a kit component, using recombination reactions. In other embodiments, recombination sites can be attached to the nucleic acid molecules of the libraries before use (e.g., by the use of a ligase, which may also be supplied with the kit). In such cases, nucleic acid molecules that contain recombination sites or primers that can be used to generate recombination sites may be supplied with the kits.

Nucleic acid molecules encoding peptides and/or fusion proteins of the invention to be supplied in kits of the invention can vary greatly. In some instances, these molecules will contain an origin of replication, at least one selectable marker, and at least one recombination site. For example, molecules supplied in kits of the invention can have four separate recombination sites that allow for insertion of sequence of interest at two different locations of a nucleic acid molecule. Other attributes of vectors supplied in kits of the invention are described elsewhere herein.

In some embodiments, the kits of the invention may comprise a plurality of containers, each container comprising one or more nucleic acid segments encoding one or more peptides and/or fusion proteins of the invention and/or one or more recombination sites and/or topoisomerase recognition sites. Segments may be provided with recombination sites such that a series of segments (e.g., two, three, four, five six, seven, eight, nine, ten, etc.) may be combined in order to construct a nucleic acid molecule of the present invention. Segments may be combined in reactions involving two or more segments (e.g., three, four, five, six, seven, eight, nine, ten, etc.). Each individual segment may be, independently of any other segment, from about 100 bp to about 35 kb in length, or from about 100 bp to about 20 kb in length, or from about 100 bp to about 10 kb in length, or from about 100 bp to about 5 kb in length, or from about 100 bp to about 2.5 kb in length, or from about 100 bp to about 1 kb in length, or from about 100 bp to about 500 bp in length. The present invention also contemplates methods for assembling and using such segments, nucleic acid molecules assembled by such methods, and compositions comprising such nucleic acid molecules.

A kit of the present invention may comprise a container containing a nucleic acid molecule encoding one or more peptides and/or fusion proteins of the invention and comprising two recombination sites that do not recombine with each other. The recombination sites may flank a selectable marker that allows selection for or against the presence of the nucleic acid molecule in a host cell or identification of a host cell containing or not containing the nucleic acid. A nucleic acid molecule to be included in a kit may comprise more than two recombination sites, for example, a nucleic acid molecule may comprise multiple pairs of recombination sites (e.g., two, three, four, five, six, seven, eight, nine, ten, etc.) where members of a pair of recombination sites do not recombine or substantially recombine with each other. In some embodiments, members of one pair of recombination sites do not recombine with members of another pair present in the same nucleic acid molecule.

Kits of the invention may comprise containers containing one or more recombination proteins. Suitable recombination proteins have been disclosed above and include, but are not limited to, Cre, Int, IHF, X is, Flp, Fis, Hin, Gin, Cin, Tn3 resolvase, ΦC31, TndX, XerC, and XerD.

Kits of the invention may also comprise one or more topoisomerase proteins and/or one or more nucleic acids comprising one or more topoisomerase recognition sequence. Suitable topoisomerases include Type IA topoisomerases, Type IB topoisomerases and/or Type II topoisomerases. Suitable topoisomerases include, but are not limited to, poxvirus topoisomerases, including vaccinia virus DNA topoisomerase I, E. coli topoisomerase III, E. coli topoisomerase I, topoisomerase III, eukaryotic topoisomerase II, archeal reverse gyrase, yeast topoisomerase III, Drosophila topoisomerase III, human topoisomerase III, Streptococcus pneumoniae topoisomerase III, bacterial gyrase, bacterial DNA topoisomerase IV, eukaryotic DNA topoisomerase II, and T-even phage encoded DNA topoisomerases, and the like. Suitable recognition sequences have been described above.

Kits of the invention may comprise one or more containers containing one or more chromatography resins. In some embodiments, a chromatography resin may comprise one or more immobilized metal ions (e.g., Ni²⁺, Co²⁺, Cu²⁺, and other divalent cations). In some embodiments, kits of the invention may comprise a container containing a chromatography resin comprising immobilized Ni²⁺.

In use, a nucleic acid molecule encoding one or more peptides and/or fusion proteins of the invention provided in a kit of the invention may be combined with a nucleic acid molecule comprising a sequence of interest using recombinational cloning. The nucleic acid molecule encoding one or more peptides and/or fusion proteins of the invention may be provided, for example, with two recombination sites that do not recombine with each other. The nucleic acid molecule comprising a sequence of interest may also be provided with two recombination sites, each of which is capable of recombining with one of the two sites present on the a nucleic acid molecule encoding one or more peptides and/or fusion proteins of the invention. In the presence of the appropriate recombination proteins, the nucleic acid molecules react to form a recombinant nucleic acid molecule containing the sequence of interest and encoding one or more peptides and/or fusion proteins of the invention. In some embodiments, the recombinant nucleic acid molecule comprises the peptide and/or fusion protein of the invention in frame with one or more coding sequence present on the sequence of interest.

Kits of the invention can also be supplied with primers. These primers will generally be designed to anneal to molecules having specific nucleotide sequences. For example, these primers can be designed for use in PCR to amplify a particular nucleic acid molecule. Further, primers supplied with kits of the invention can be sequencing primers designed to hybridize to vector sequences. Thus, such primers will generally be supplied as part of a kit for sequencing nucleic acid molecules that have been inserted into a vector.

One or more buffers (e.g., one, two, three, four, five, eight, ten, fifteen) may be supplied in kits of the invention. These buffers may be supplied at a working concentrations or may be supplied in concentrated form and then diluted to the working concentrations. These buffers will often contain salt, metal ions, co-factors, metal ion chelating agents, etc. for the enhancement of activities of the stabilization of either the buffer itself or molecules in the buffer. Further, these buffers may be supplied in dried or aqueous forms. When buffers are supplied in a dried form, they will generally be dissolved in water prior to use.

Kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

Kits of the invention may comprise one or more pages of written instructions for carrying out the methods of the invention. For example, instructions may comprise method steps necessary to carryout recombinational cloning of an ORF provided with recombination sites and a vector also comprising recombination sites and optionally further comprising one or more functional sequences.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Method for Analyzing Requirements for Affinity Peptide Design

The NCBI Molecular Modeling Database (MMDB) was queried with the terms “nickel”, “copper”, “zinc”, etc. A particular query would yield structural data for a particular set of proteins. For instance, the query “nickel” generated the list of proteins below in Table 19 for which structural data was available.

TABLE 19
List of proteins generated by query “nickel.”

1IE7 Phosphate Inhibited Bacillus pasteurii Urease Crystal Structure
1ES7 Complex Between Bmp-2 And Two Bmp Receptor Ia Ectodomains
1E5K Crystal Structure Of The Molybdenum Cofactor Biosynthesis Protein
Moba (Protein Fa) From Escherichia Coli At Near Atomic Resolution
1EJV Crystal Structure Of The H320q Variant Of Klebsiella aerogenes Urease
1EJU Crystal Structure Of The H320n Variant Of Klebsiella Aerogenes Urease
1EJT Crystal Structure Of The H219q Variant Of Klebsiella aerogenes Urease
1EJS Crystal Structure Of The H219n Variant Of Klebsiella aerogenes Urease
1EJR Crystal Structure Of The D221a Variant Of Klebsiella aerogenes Urease
1F5T Diphtheria Tox Repressor (C102d Mutant) Complexed With Nickel And Dtxr Consensus
Binding Sequence
1CFZ Hydrogenase Maturating Endopeptidase Hybd From E. Coli
4UBP Structure Of Bacillus Pasteurii Urease Inhibited With Acetohydroxamic Acid At 1.55
\acute{Å} Resolution
3UBP Diamidophosphate Inhibited Bacillus pasteurii Urease
2UBP Structure Of Native Urease From Bacillus pasteurii
1UBP Crystal Structure Of Urease From Bacillus pasteurii Inhibited With
Beta-Mercaptoethanol At 1.65 Angstroms Resolution
1BSZ Peptide Deformylase As Fe²⁺ Containing Form (Native) In Complex With Inhibitor
Polyethylene Glycol
1BS8 Peptide Deformylase As Zn²⁺ Containing Form In Complex With Tripeptide
Met-Ala-Ser
1BS7 Peptide Deformylase As Ni²⁺ Containing Form
1BS6 Peptide Deformylase As Ni²⁺ Containing Form In Complex With
Tripeptide Met-Ala-Ser
1BS5 Peptide Deformylase As Zn²⁺ Containing Form
1BS4 Peptide Deformylase As Zn2+ Containing Form (Native) In Complex
With Inhibitor Polyethylene Glycol
446D Structure Of The Oligonucleotide D(Cgtatatacg) As A Site Specific
Complex With Nickel Ions
2TDX Diphtheria Tox Repressor (C102d Mutant) Complexed With Nickel
1DDN Diphtheria Tox Repressor (C102d Mutant) Complexed With Nickel
And With Tox Dna Operator
2FRV Crystal Structure Of The Oxidized Form Of Ni—Fe Hydrogenase
1A5O K217c Variant Of Klebsiella aerogenes Urease, Chemically Rescued
By Formate And Nickel
1A5N K217a Variant Of Klebsiella aerogenes Urease, Chemically Rescued
By Formate And Nickel
1A5M K217a Variant Of Klebsiella aerogenes Urease
1A5L K217c Variant Of Klebsiella aerogenes Urease
1A5K K217e Variant Of Klebsiella aerogenes Urease
1AQP Ribonuclease A Copper Complex
2DEF Peptide Deformylase Catalytic Core (Residues 1-147), Nmr, 20 Structures
1FWJ Klebsiella aerogenes Urease, Native
1FWI Klebsiella aerogenes Urease, H134a Variant
1FWH Klebsiella aerogenes Urease, C319y Variant
1FWG Klebsiella aerogenes Urease, C319s Variant
1FWF Klebsiella aerogenes Urease, C319d Variant
1FEW Klebsiella aerogenes Urease, C319a Variant With Acetohydroxamic Acid (Aha) Bound
1FWD Klebsiella aerogenes Urease, C319a Variant At pH 9.4
1FWC Klebsiella aerogenes Urease, C319a Variant At pH 8.5
1FWB Klebsiella aerogenes Urease, C319a Variant At pH 6.5
1FWA Klebsiella aerogenes Urease, C319a Variant At pH 7.5
1FRV Crystal Structure Of The Oxidized Form Of Ni—Fe Hydrogenase
1SLW Rat Anionic N143h, E151h Trypsin Complexed To A86h Ecotin; Nickel-Bound
1KRA Apoenzyme, Nickel Metalloenzyme Mol_id: 1; Molecule: Urease;
Chain: A, B, C; Ec: 3.5.1.5
1KRB Active Site Mutant, Nickel Metalloenzyme Mol_id: 1; Molecule:
Urease; Chain: A, B, C; Ec: 3.5.1.5; Mutation: H(C 219)a; Heterogen: Carbon
Dioxide; Heterogen: Nickel
1KRC Active Site Mutant, Nickel Metalloenzyme Mol_id: 1; Molecule:
Urease; Chain: A, B, C; Ec: 3.5.1.5; Mutation: H(C 320)a; Heterogen: Carbon
Dioxide; Heterogen: Nickel
2KAU Klebsiella aerogenes Urease; Ec: 3.5.1.5; Synonyms: Urea
Amidohydrolase, Urease; Engineered
1NZR Azurin Mutant With Trp 48 Replaced By Met (W48m)
1SCR Concanavalin A (Nickel Substituted For Manganese)
1IAE Astacin (E.C.3.4.24.21) With Zinc Replaced By Nickel(II)
1IAC Astacin (E.C.3.4.24.21) With Zinc Replaced By Mercury(II)
1IAB Astacin (E.C.3.4.24.21) With Zinc Replaced By Cobalt(II)
1IAA Astacin (E.C.3.4.24.21) With Zinc Replaced By Copper(II)
1RZE Carbonic Anhydrase Ii (E.C.4.2.1.1) With Zinc Replaced By Nickel(II)

The subset of the data shown in Table 20 was downloaded and saved in format suitable for 3-D visualization using publicly available software. Those skilled in the art will appreciate that any other means (e.g., computer programs, etc.) that permit the visualization of protein structures may be used in the practice of the invention.

TABLE 20
1ES7 Complex Between Bmp-2 And Two Bmp Receptor Ia Ectodomains
1E5K Crystal Structure Of The Molybdenum Cofactor Biosynthesis
Protein Moba (Protein Fa) From Escherichia coli At Near
Atomic Resolution
1CFZ Hydrogenase Maturating Endopeptidase Hybd From E. coli
2UBP Structure Of Native Urease From Bacillus pasteurii
2TDX Diphtheria Tox Repressor (C102d Mutant) Complexed With Nickel
1DDN Diphtheria Tox Repressor (C102d Mutant) Complexed With Nickel
And With Tox Dna Operator
2FRV Crystal Structure Of The Oxidized Form Of Ni—Fe Hydrogenase
1AQP Ribonuclease A Copper Complex
1FRV Crystal Structure Of The Oxidized Form Of Ni—Fe Hydrogenase
1SLW Rat Anionic N143h, E151h Trypsin Complexed To A86h Ecotin;
Nickel-Bound
1NZR Azurin Mutant With Trp 48 Replaced By Met (W48m)
1SCR Concanavalin A (Nickel Substituted For Manganese)
1IAE Astacin (E.C.3.4.24.21) With Zinc Replaced By Nickel(II)
1RZE Carbonic Anhydrase Ii (E.C.4.2.1.1) With Zinc
Replaced By Nickel(II)

The SwissPDBViewer program generates a three dimensionally rotatable, translatable, and magnifiable representation of a protein, as well as other atoms present in the crystal from which the coordinates were derived. Using functions of the software, nickel atoms were located within the virtual three dimensional space defined by the protein. The image was modified to display only amino acid residues present in an approximately 4-6 Å sphere around the metal atom using another function of the program. The spatial orientation and relationship with respect to the metal atom of such residues so identified were noted, and one or more images captured. This process was repeated for a sufficient number of proteins so that testable predictions could be made about the structure of a peptide capable of coordinating a particular metal atom. A list of selected coordinate files appears in Table 21.

TABLE 21

| | |
|---|---|
| 1CFZ Hydrogenase Maturating | 2TDX Diphtheria Tox Repressor |
| Endopeptidase Hybd From E. coli | (C102d Mutant) Complexed |
| | With Nickel |
| 1DDN Diphtheria Tox Repressor | 1SLW Rat Anionic N143h, |
| (C102d Mutant) Complexed | E151h Trypsin Complexed |
| With Nickel And With | To A86h Ecotin; Nickel-Bound |
| Tox Dna Operator | |
| 1NZR Azurin Mutant With Trp | 1RZE Carbonic Anhydrase II |
| 48 Replaced By Met | (E.C.4.2.1.1) With |
| | Zinc Replaced By Nickel(II) |
| 1BS7 Peptide Deformylase | |
| As Ni2+ Containing | |
| Form | |

Several observations were made regarding the structure of proteic nickel coordination spheres:
Each of H, C, M, D, E, Q, Y, G residues were present in at least one structure and did not exhibit any positional bias, relative to the primary structure of the protein;
Histidines, when more than one was present in a coordination sphere, were not found adjacent in the primary structure of the protein. In all cases, they were interspersed by one to many residues. Thus, adjacent histidines do not appear to be a requirement for nickel coordination;
acidic amino acids (D and/or E) were almost always present in a coordination sphere;
sulfur-containing residues (M and/or C) were often present in a coordination sphere; and
acidic (D and/or E) and sulfur-containing (M and/or C) residues rarely occur together in a coordination sphere.

Based upon these observations, peptide sequences embodying one or more of the above properties were inferred from the structural data. Because there was no apparent positional bias, the predicted peptide sequences were permuted to encompass possible structural variations. Thus, a peptide of the invention may comprise one or more amino acids drawn from the group: G, A, V, L, I, P, F, Y, W, S, T, N, Q, C, M, D, E, H, K, R. In a preferred embodiment, a peptide of the invention may comprise one or more amino acids drawn from the group: H, C, M, D, E, Q, Y, or G. In particular, peptides of the invention are those that do not contain two or more adjacent histidines.

### Example 2

### Binding of Peptides to Nickel Matrices Predicted from Structural Data

Peptides were predicted using methods of the invention and were chemically synthesized with an N-terminal FITC moiety. The peptides were then tested for their ability to bind a nickel chromatography matrix. The following peptides were tested:

| | | | |
|---|---|---|---|
| HHHHHH | HGDGH | HGGDGGH | HGSDGSH |
| (SEQ ID NO: 622) | (SEQ ID NO: 43) | (SEQ ID NO: 88) | (SEQ ID NO: 115) |
| HSGDSGH | HGDSH | HSDGH | DGHGD |
| (SEQ ID NO: 142) | (SEQ ID NO: 169) | (SEQ ID NO: 196) | (SEQ ID NO: 232) |
| DGHGE | DGGHGGD | DGGHGGE | DGHSD |
| (SEQ ID NO: 233) | (SEQ ID NO: 268) | (SEQ ID NO: 269) | (SEQ ID NO: 295) |
| DGHSE | DGGHSSD | DGGHSSE | EGGHSSD |
| (SEQ ID NO: 296) | (SEQ ID NO: 323) | (SEQ ID NO: 324) | (SEQ ID NO: 326) |
| EGGHSSE | HGEGH | HGGEGGH | HGSEGSH |
| (SEQ ID NO: 327) | (SEQ ID NO: 52) | (SEQ ID NO: 97) | (SEQ ID NO: 124) |
| HSGESGH | HGESH | HSEGH | GSHDHG |
| (SEQ ID NO: 151) | (SEQ ID NO: 178) | (SEQ ID NO: 205) | (SEQ ID NO: 631) |
| SHDHG | HDHG | HDH | HDGHT |
| (SEQ ID NO: 623) | (SEQ ID NO: 626) | | (SEQ ID NO: 632) |
| HDGHS | SHDGH | THDGH | SHDGSH |
| (SEQ ID NO: 624) | (SEQ ID NO: 627) | (SEQ ID NO: 629) | (SEQ ID NO: 633) |
| THDGTH | HGAHDHG | GSHGAH | |
| (SEQ ID NO: 625) | (SEQ ID NO: 628) | (SEQ ID NO: 630) | |

The following buffers were used:
Buffer A: 50 mM NaPO₄, pH 7.5, 5% glycerol, 500 mM NaCl, 5 mM imidazole;
Buffer B: 50 mM NaPO₄, pH 7.5, 5% glycerol, 500 mM NaCl, 20 mM imidazole;
Buffer C: 50 mM NaPO₄, pH 7.5, 5% glycerol, 500 mM NaCl, 500 mM imidazole;

The peptides were tested for ability to bind Ni²⁺ as follows:
1) peptide vials were allowed to warm to room temperature in the dark;
2) 2 mgs of each peptide was weighed into an Eppendorf tube in the dark;
3) vials were tightly closed and sealed with Parafilm, and returned to freezer in the dark
4) peptides were dissolved in 1 ml buffer A (stock peptide, 2 mg/ml);
5) 30 μl of each peptide solution was transferred to a new tube, 450 μl of buffer A was added and mixed resulting in a diluted peptide concentration of 125 μg/ml;
6) a Swellgel bead was placed into a mini-spin column (Biorad) with the end cap sealed;
7) 400 μl of diluted peptide was added to the column and incubated at room temperature (RT) for 5 minutes;
8) samples were vortexed briefly, end caps were removed and, columns were centrifuged for 2 minutes at 1,000 rpm, the solution collected was termed flow through (FT);
9) 400 μl Buffer A was added, samples were vortexed briefly, centrifuged for 2 minutes at 1,000 rpm, the solution collected was termed second flow through (FT2);
10) step 9 was repeated twice with 400 μl Buffer B, solutions were collected and termed wash (W) and second wash (W2);
11) 200 μl Buffer C was added and incubated for 5 minutes at RT;
12) samples were vortexed briefly and centrifuged for 2 minutes at 1,000 rpm;
13) an additional 200 μl Buffer C was added, samples were vortexed briefly and centrifuged for 2 minutes at 1,000 rpm into the same tube as 12, the combined solutions were termed eluant (E);
14) a 100 μl sample of each collected solution was added to a well of a microtiter plate, the sample of the solution loaded onto the column and strong eluants were diluted 1:4 with water;
15) plates were read and quantified using Typhoon microplate reader on the fluorescein setting, 500 PMV.

Results of the above analysis for the indicated peptides are provided in Table 22.

TABLE 22
Ni Affinity peptide binding

| | | % | | | |
|---|---|---|---|---|---|
| Ni# | Sequence | recov | % FT | % Wash | % Elute |
| 1 | HHHHHH (SEQ ID NO: 622) | 38 | 2 | 2 | 96 |
| 34 | HGAHDHG (SEQ ID NO: 628) | 97 | 5 | 8 | 87 |
| 24 | GSHDHG (SEQ ID NO: 631) | 89 | 8 | 20 | 72 |
| 35 | GSHGAH (SEQ ID NO: 630) | 100 | 15 | 16 | 69 |
| 25 | SHDHG (SEQ ID NO: 623) | 100 | 17 | 15 | 69 |
| 27 | HDH | 83 | 14 | 17 | 68 |
| 28 | HDGHT (SEQ ID NO: 632) | 98 | 11 | 24 | 66 |
| 8 | DGHGD (SEQ ID NO: 232) | 92 | 14 | 20 | 66 |
| 26 | HDHG (SEQ ID NO: 626) | 79 | 16 | 18 | 66 |
| 29 | HDGHS (SEQ ID NO: 624) | 90 | 9 | 26 | 65 |
| 2 | HGDGH (SEQ ID NO: 43) | 76 | 14 | 21 | 65 |
| 18 | HGEGH (SEQ ID NO: 52) | 89 | 10 | 28 | 62 |
| 7 | HSDGH (SEQ ID NO: 196) | 82 | 19 | 23 | 58 |
| 22 | HGESH (SEQ ID NO: 178) | 91 | 15 | 30 | 56 |
| 31 | THDGH (SEQ ID NO: 629) | 99 | 20 | 24 | 56 |
| 19 | HGGEGGH (SEQ ID NO: 97) | 82 | 20 | 25 | 55 |
| 20 | HGSEGSH (SEQ ID NO: 124) | 93 | 15 | 31 | 54 |
| 4 | HGSDGSH (SEQ ID NO: 115) | 89 | 22 | 26 | 53 |
| 6 | HGDSH (SEQ ID NO: 169) | 100 | 24 | 22 | 53 |
| 3 | HGGDGGH (SEQ ID NO: 88) | 100 | 30 | 19 | 51 |
| 32 | SHDGSH (SEQ ID NO: 633) | 100 | 13 | 37 | 50 |
| 33 | THDGTH (SEQ ID NO: 625) | 100 | 18 | 31 | 50 |
| 23 | HSEGH (SEQ ID NO: 205) | 99 | 27 | 23 | 50 |
| 21 | HSGESGH (SEQ ID NO: 151) | 97 | 27 | 30 | 44 |
| 5 | HSGDSGH (SEQ ID NO: 142) | 84 | 29 | 28 | 43 |
| 13 | DGHSE (SEQ ID NO: 296) | 100 | 63 | 27 | 10 |
| 12 | DGHSD (SEQ ID NO: 295) | 96 | 54 | 37 | 9 |
| 9 | DGHGE (SEQ ID NO: 233) | 78 | 63 | 29 | 8 |
| 14 | DGGHSSD (SEQ ID NO: 323) | 100 | 65 | 28 | 8 |
| 10 | DGGHGGD (SEQ ID NO: 268) | 93 | 71 | 23 | 6 |
| 15 | DGGHSSE (SEQ ID NO: 324) | 72 | 60 | 35 | 5 |
| 16 | EGGHSSD (SEQ ID NO: 326) | 80 | 68 | 28 | 4 |
| 17 | EGGHSSE (SEQ ID NO: 327) | 96 | 70 | 26 | 4 |

| | | |
|---|---|---|
| 30 | SHDGH (SEQ ID NO: 627) | NOT TESTED |
| 11 | DGGHGGE (SEQ ID NO: 269) | NOT TESTED |

### Example 3

### Binding of Predicted Peptides to Nickel Matrices

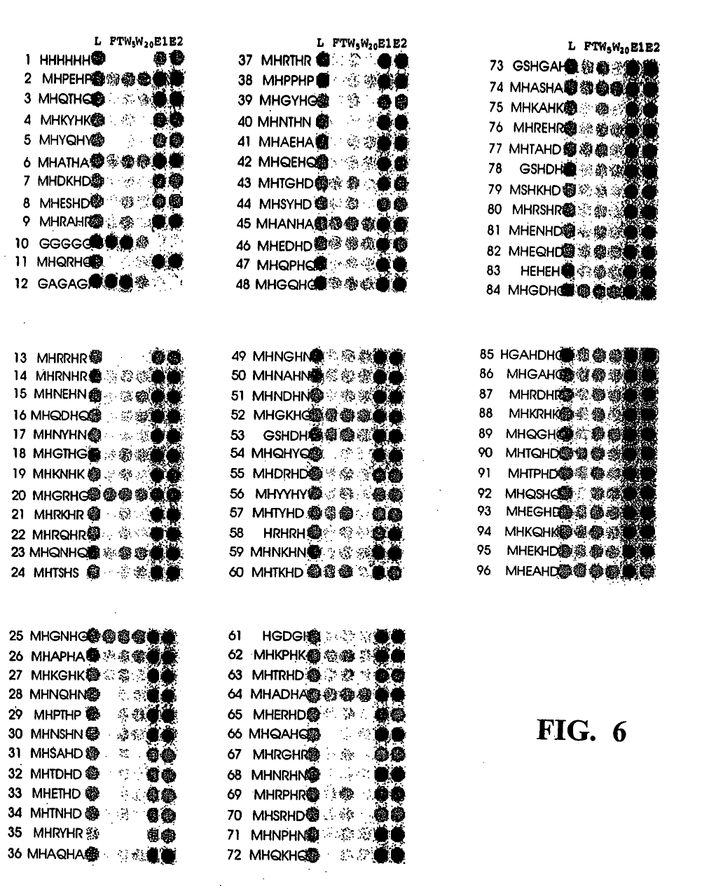

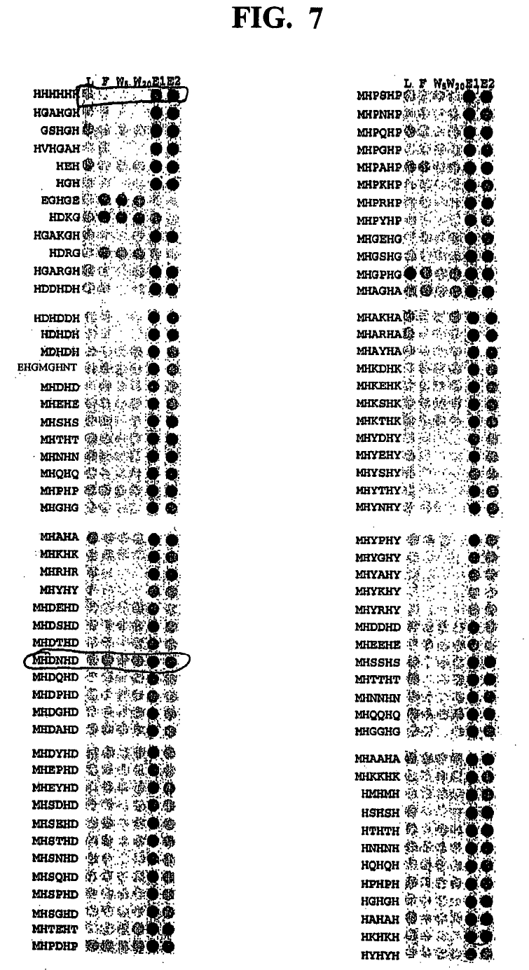

Peptides were chemically synthesized with an N-terminal FITC moiety. The peptides were then tested for their ability to bind a nickel chromatography matrix essentially as in Example 2 except the assays were performed in a high throughput protocol using a 96-well plate format. Two washes of 200 μl were preformed with each of buffers A and B and each wash was kept separate. Two elutions of 200 μl were performed with buffer C and kept separate. Solutions were analyzed using a Typhoon Phosphorimager (Molecular Dynamics). FIGS. 6 and 7 show the results of these experiments for the indicated peptides and the data is presented in tabular form below. For the sake of brevity, FIGS. 6 and 7 show only one of the wash solutions for each of buffer A (indicated as W₅) and buffer B (indicated as W₂₀). The results for the indicated peptides are shown in FIGS. 6 and 7 and are presented in tabular form below in Tables 23-25. The following peptides were tested:

| MHDDHD | MHEEHE | MHSSHS | MHTTHT | MHNNHN | MHQQHQ |
|---|---|---|---|---|---|
| (SEQ ID | (SEQ ID | (SEQ ID | (SEQ ID | (SEQ ID | (SEQ ID |
| NO: 443) | NO: 455) | NO: 467) | NO: 479) | NO: 491) | NO: 503) |
| MHDEHD | MHEDHD | MHSDHD | MHTEHT | MHNDHN | MHQDHQ |
| (SEQ ID | (SEQ ID | (SEQ ID | (SEQ ID | (SEQ ID | (SEQ ID |
| NO: 444) | NO: 634) | NO: 645) | NO: 480) | NO: 492) | NO: 504) |
| MHDSHD | MHESHD | MHSEHD | MHTSHS | MHNEHN | MHQEHQ |
| (SEQ ID | (SEQ ID | (SEQ ID | (SEQ ID | (SEQ ID | (SEQ ID |
| NO: 445) | NO: 635) | NO: 646) | NO: 656) | NO: 493) | NO: 505) |
| MHDTHD | MHETHD | MHSTHD | MHTDHD | MHNSHN | MHQSHQ |
| (SEQ ID | (SEQ ID | (SEQ ID | (SEQ ID | (SEQ ID | (SEQ ID |
| NO: 446) | NO: 636) | NO: 647) | NO: 657) | NO: 494) | NO: 506) |
| MHDNHD | MHENHD | MHSNHD | MHTNHD | MHNTHN | MHQTHQ |
| (SEQ ID | (SEQ ID | (SEQ ID | (SEQ ID | (SEQ ID | (SEQ ID |
| NO: 447) | NO: 637) | NO: 648) | NO: 658) | NO: 495) | NO: 507) |
| MHDQHD | MHEQHD | MHSQHD | MHTQHD | MHNQHN | MHQNHQ |
| (SEQ ID | (SEQ ID | (SEQ ID | (SEQ ID | (SEQ ID | (SEQ ID |
| NO: 448) | NO: 638) | NO: 649) | NO: 659) | NO: 496) | NO: 508) |
| MHDPHD | MHEPHD | MHSPHD | MHTPHD | MHNPHN | MHQPHQ |
| (SEQ ID | (SEQ ID | (SEQ ID | (SEQ ID | (SEQ ID | (SEQ ID |
| NO: 449) | NO: 639) | NO: 650) | NO: 660) | NO: 497) | NO: 509) |
| MHDGHD | MHEGHD | MHSGHD | MHTGHD | MHNGHN | MHQGHQ |
| (SEQ ID | (SEQ ID | (SEQ ID | (SEQ ID | (SEQ ID | (SEQ ID |
| NO: 450) | NO: 640) | NO: 651) | NO: 661) | NO: 498) | NO: 510) |
| MHDAHD | MHEAHD | MHSAHD | MHTAHD | MHNAHN | MHQAHQ |
| (SEQ ID | (SEQ ID | (SEQ ID | (SEQ ID | (SEQ ID | (SEQ ID |
| NO: 451) | NO: 641) | NO: 652) | NO: 662) | NO: 499) | NO: 511) |
| MHDKHD | MHEKHD | MHSKHD | MHTKHD | MHNKHN | MHQKHQ |
| (SEQ ID | (SEQ ID | (SEQ ID | (SEQ ID | (SEQ ID | (SEQ ID |
| NO: 452) | NO: 642) | NO: 653) | NO: 663) | NO: 500) | NO: 512) |
| MHDRHD | MHERHD | MHSRHD | MHTRHD | MHNRHN | MHQRHQ |
| (SEQ ID | (SEQ ID | (SEQ ID | (SEQ ID | (SEQ ID | (SEQ ID |
| NO: 453) | NO: 643) | NO: 654) | NO: 664) | NO: 501) | NO: 513) |
| MHDYHD | MHEYHD | MHSYHD | MHTYHD | MHNYHN | MHQHYQ |
| (SEQ ID | (SEQ ID | (SEQ ID | (SEQ ID | (SEQ ID | (SEQ ID |
| NO: 454) | NO: 644) | NO: 655) | NO: 665) | NO: 502) | NO: 514) |
| MHPPHP | MHGGHG | MHAAHA | MHKKHK | MHRRHR | MHYYHY |
| (SEQ ID | (SEQ ID | (SEQ ID | (SEQ ID | (SEQ ID | (SEQ ID |
| NO: 515) | NO: 527) | NO: 539) | NO: 551) | NO: 563) | NO: 575) |
| MHPDHP | MHGDHG | MHADHA | MHKDHK | MHRDHR | MHYDHY |
| (SEQ ID | (SEQ ID | (SEQ ID | (SEQ ID | (SEQ ID | (SEQ ID |
| NO: 516) | NO: 528) | NO: 540) | NO: 552) | NO: 564) | NO: 576) |
| MHPEHP | MHGEHG | MHAEHA | MHKEHK | MHREHR | MHYEHY |
| (SEQ ID | (SEQ ID | (SEQ ID | (SEQ ID | (SEQ ID | (SEQ ID |
| NO: 517) | NO: 529) | NO: 541) | NO: 553) | NO: 565) | NO: 577) |
| MHPSHP | MHGSHG | MHASHA | MHKSHK | MHRSHR | MHYSHY |
| (SEQ ID | (SEQ ID | (SEQ ID | (SEQ ID | (SEQ ID | (SEQ ID |
| NO: 518) | NO: 530) | NO: 542) | NO: 554) | NO: 566) | NO: 578) |
| MHPTHP | MHGTHG | MHATHA | MHKTHK | MHRTHR | MHYTHY |
| (SEQ ID | (SEQ ID | (SEQ ID | (SEQ ID | (SEQ ID | (SEQ ID |
| NO: 519) | NO: 531) | NO: 543) | NO: 555) | NO: 567) | NO: 579) |
| MHPNHP | MHGNHG | MHANHA | MHKNHK | MHRNHR | MHYNHY |
| (SEQ ID | (SEQ ID | (SEQ ID | (SEQ ID | (SEQ ID | (SEQ ID |
| NO: 520) | NO: 532) | NO: 544) | NO: 556) | NO: 568) | NO: 580) |
| MHPQHP | MHGQHG | MHAQHA | MHKQHK | MHRQHR | MHYQHY |
| (SEQ ID | (SEQ ID | (SEQ ID | (SEQ ID | (SEQ ID | (SEQ ID |
| NO: 521) | NO: 533) | NO: 545) | NO: 557) | NO: 569) | NO: 581) |
| MHPGHP | MHGPHG | MHAPHA | MHKPHK | MHRPHR | MHYPHY |
| (SEQ ID | (SEQ ID | (SEQ ID | (SEQ ID | (SEQ ID | (SEQ ID |
| NO: 522) | NO: 534) | NO: 546) | NO: 558) | NO: 570) | NO: 582) |
| MHPAHP | MHGAHG | MHAGHA | MHKGHK | MHRGHR | MHYGHY |
| (SEQ ID | (SEQ ID | (SEQ ID | (SEQ ID | (SEQ ID | (SEQ ID |
| NO: 523) | NO: 535) | NO: 547) | NO: 559) | NO: 571) | NO: 583) |
| MHPKHP | MHGKHG | MHAKHA | MHKAHK | MHRAHR | MHYAHY |
| (SEQ ID | (SEQ ID | (SEQ ID | (SEQ ID | (SEQ ID | (SEQ ID |
| NO: 524) | NO: 536) | NO: 548) | NO: 560) | NO: 572) | NO: 584) |
| MHPRHP | MHGRHG | MHARHA | MHKRHK | MHRKHR | MHYKHY |
| (SEQ ID | (SEQ ID | (SEQ ID | (SEQ ID | (SEQ ID | (SEQ ID |
| NO: 525) | NO: 537) | NO: 549) | NO: 561) | NO: 573) | NO: 585) |
| MHPYHP | MHGYHG | MHAYHA | MHKYHK | MHRYHR | MHYRHY |
| (SEQ ID | (SEQ ID | (SEQ ID | (SEQ ID | (SEQ ID | (SEQ ID |
| NO: 526) | NO: 538) | NO: 550) | NO: 562) | NO: 574) | NO: 586) |
| HDHDH | EHGMGHNT | MHYHY | HDHDDH | MHRHR | HDDHDH |
| (SEQ ID | (SEQ ID NO: | (SEQ ID | (SEQ ID | (SEQ ID | (SEQ ID |
| NO: 350) | 606) | NO: 589) | NO: 667) | NO: 595) | NO: 367) |
| HEHEH | MDHDH | HGAHGH | HMHMH | MHKHK | HGARGH |
| (SEQ ID | (SEQ ID NO: | (SEQ ID | (SEQ ID | (SEQ ID | (SEQ ID |
| NO: 351) | 603) | NO: 363) | NO: 362) | NO: 594) | NO: 602) |
| HSHSH | MHDHD | GSHDH | MHAHA | HDRG | HAHAH |
| (SEQ ID | (SEQ ID NO: | (SEQ ID | (SEQ ID | (SEQ ID | (SEQ ID |
| NO: 352) | 597) | NO: 604) | NO: 593) | NO: 668) | NO: 358) |
| HTHTH | MHEHE | GSHGH | MHGHG | HGAKGH | HKHKH |
| (SEQ ID | (SEQ ID NO: | (SEQ ID | (SEQ ID | (SEQ ID | (SEQ ID |
| NO: 353) | 598) | NO: 605) | NO: 592) | NO: 601) | NO: 359) |
| HNHNH | MHSHS | HVHGAH | MHPHP | HDKG | HRHRH |
| (SEQ ID | (SEQ ID NO: | (SEQ ID | (SEQ ID | (SEQ ID | (SEQ ID |
| NO: 354) | 599) | NO: 666) | NO: 591) | NO: 669) | NO: 360) |
| HQHQH | MHTHT | HEH | MHQHQ | EGHGE | HYHYH |
| (SEQ ID | (SEQ ID NO: | | (SEQ ID | (SEQ ID | (SEQ ID |
| NO: 355) | 600) | | NO: 590) | NO: 236) | NO: 361) |
| HPHPH | MHNHN | HGH | HGHGH | | |
| (SEQ ID | (SEQ ID NO: | | (SEQ ID | | |
| NO: 356) | 596) | | NO: 357) | | |

TABLE 23

| | P# | Peptide | Recov | % FT | % W | % E |
|---|---|---|---|---|---|---|
| 1 | 1 | HHHHHH | 46% | 1% | 1% | 98% |
| | | (SEQ ID NO: 622) | | | | |
| 14 | 53 | HDHDH | 95% | 3% | 3% | 94% |
| | | (SEQ ID NO: 350) | | | | |
| 2 | 40 | HGAHGH | 91% | 3% | 3% | 94% |
| | | (SEQ ID NO: 363) | | | | |
| 4 | 43 | HVHGAH | 103% | 4% | 3% | 94% |
| | | (SEQ ID NO: 666) | | | | |
| 27 | 66 | MHRHR | 120% | 4% | 4% | 92% |
| | | (SEQ ID NO: 595) | | | | |
| 77 | 199 | MHYRHY | 80% | 5% | 4% | 91% |
| | | (SEQ ID NO: 586) | | | | |
| 12 | 51 | HDDHDH | 106% | 4% | 6% | 91% |
| | | (SEQ ID NO: 367) | | | | |
| 88 | 214 | HSHSH | 89% | 5% | 5% | 90% |
| | | (SEQ ID NO: 352) | | | | |
| 95 | 221 | HKHKH | 78% | 5% | 5% | 90% |
| | | (SEQ ID NO: 359) | | | | |
| 13 | 52 | HDHDDH | 103% | 4% | 6% | 90% |
| | | (SEQ ID NO: 667) | | | | |
| 6 | 45 | HGH | 103% | 3% | 8% | 89% |
| 89 | 215 | HTHTH | 90% | 5% | 6% | 89% |
| | | (SEQ ID NO: 353) | | | | |
| 19 | 58 | MHSHS | 100% | 5% | 6% | 88% |
| | | (SEQ ID NO: 599) | | | | |
| 71 | 192 | MHYTHY | 86% | 6% | 6% | 88% |
| | | (SEQ ID NO: 579) | | | | |
| 20 | 59 | MHTHT | 99% | 5% | 7% | 88% |
| | | (SEQ ID NO: 600) | | | | |
| 74 | 196 | MHYGHY | 96% | 7% | 5% | 88% |
| | | (SEQ ID NO: 583) | | | | |
| 28 | 67 | MHYHY | 82% | 9% | 3% | 88% |
| | | (SEQ ID NO: 589) | | | | |
| 75 | 197 | MHYAHY | 118% | 7% | 5% | 88% |
| | | (SEQ ID NO: 584) | | | | |
| 21 | 60 | MHNHN | 97% | 5% | 7% | 88% |
| | | (SEQ ID NO: 596) | | | | |
| 93 | 219 | HGHGH | 92% | 7% | 6% | 88% |
| | | (SEQ ID NO: 357) | | | | |
| 26 | 65 | MHKHK | 109% | 7% | 6% | 87% |
| | | (SEQ ID NO: 594) | | | | |
| 76 | 198 | MHYKHY | 123% | 8% | 5% | 87% |
| | | (SEQ ID NO: 585) | | | | |
| 72 | 193 | MHYNHY | 121% | 8% | 5% | 87% |
| | | (SEQ ID NO: 580) | | | | |
| 25 | 64 | MHAHA | 94% | 4% | 9% | 87% |
| | | (SEQ ID NO: 593) | | | | |
| 94 | 220 | HAHAH | 83% | 8% | 6% | 86% |
| | | (SEQ ID NO: 358) | | | | |
| 90 | 216 | HNHNH | 92% | 7% | 6% | 86% |
| | | (SEQ ID NO: 354) | | | | |
| 3 | 42 | GSHGH | 105% | 5% | 9% | 86% |
| | | (SEQ ID NO: 605) | | | | |
| 22 | 61 | MHQHQ | 114% | 5% | 9% | 85% |
| | | (SEQ ID NO: 590) | | | | |
| 73 | 195 | MHYPHY | 99% | 9% | 6% | 85% |
| | | (SEQ ID NO: 582) | | | | |
| 70 | 191 | MHYSHY | 116% | 9% | 6% | 85% |
| | | (SEQ ID NO: 578) | | | | |
| 24 | 63 | MHGHG | 129% | 6% | 9% | 85% |
| | | (SEQ ID NO: 592) | | | | |
| 63 | 166 | MHAYHA | 70% | 5% | 11% | 84% |
| | | (SEQ ID NO: 550) | | | | |
| 69 | 190 | MHYEHY | 112% | 7% | 9% | 84% |
| | | (SEQ ID NO: 577) | | | | |
| 87 | 212 | HMHMH | 114% | 9% | 8% | 83% |
| | | (SEQ ID NO: 362) | | | | |
| 84 | 207 | MHGGHG | 103% | 5% | 12% | 83% |
| | | (SEQ ID NO: 527) | | | | |
| 11 | 50 | HGARGH | 107% | 4% | 13% | 83% |
| | | (SEQ ID NO: 602) | | | | |
| 9 | 48 | HGAKGH | 108% | 4% | 13% | 82% |
| | | (SEQ ID NO: 601) | | | | |
| 56 | 144 | MHPYHP | 96% | 5% | 12% | 82% |
| | | (SEQ ID NO: 526) | | | | |
| 23 | 62 | MHPHP | 93% | 9% | 8% | 82% |
| | | (SEQ ID NO: 591) | | | | |
| 62 | 165 | MHARHA | 84% | 5% | 14% | 81% |
| | | (SEQ ID NO: 549) | | | | |
| 58 | 147 | MHGSHG | 98% | 6% | 13% | 81% |
| | | (SEQ ID NO: 530) | | | | |
| 82 | 204 | MHNNHN | 101% | 6% | 13% | 81% |
| | | (SEQ ID NO: 491) | | | | |
| 92 | 218 | HPHPH | 94% | 10% | 9% | 81% |
| | | (SEQ ID NO: 356) | | | | |
| 81 | 203 | MHTTHT | 89% | 3% | 16% | 81% |
| | | (SEQ ID NO: 479) | | | | |
| 55 | 143 | MHPRHP | 87% | 6% | 13% | 80% |
| | | (SEQ ID NO: 525) | | | | |
| 80 | 202 | MHSSHS | 96% | 5% | 15% | 80% |
| | | (SEQ ID NO: 467) | | | | |
| 68 | 189 | MHYDHY | 109% | 12% | 8% | 80% |
| | | (SEQ ID NO: 576) | | | | |
| 5 | 44 | HEH | 84% | 7% | 13% | 79% |
| 66 | 169 | MHKSHKI | 99% | 7% | 13% | 79% |
| | | (SEQ ID NO: 670) | | | | |
| 86 | 209 | MHKKHK | 95% | 6% | 15% | 79% |
| | | (SEQ ID NO: 551) | | | | |

TABLE 24

| | P# | Peptide | Recov | % FT | % W | % E |
|---|---|---|---|---|---|---|
| 85 | 208 | MHAAHA | 99% | 6% | 16% | 77% |
| | | (SEQ ID NO: 539) | | | | |
| 49 | 136 | MHPSHP | 94% | 7% | 16% | 77% |
| | | (SEQ ID NO: 518) | | | | |
| 54 | 142 | MHPKHP | 102% | 7% | 16% | 77% |
| | | (SEQ ID NO: 524) | | | | |
| 67 | 170 | MHKTHK | 101% | 10% | 14% | 76% |
| | | (SEQ ID NO: 555) | | | | |
| 91 | 217 | HQHQH | 94% | 13% | 11% | 76% |
| | | (SEQ ID NO: 355) | | | | |
| 50 | 138 | MHPNHP | 97% | 7% | 17% | 76% |
| | | (SEQ ID NO: 520) | | | | |
| 18 | 57 | MHEHE | 124% | 9% | 15% | 76% |
| | | (SEQ ID NO: 598) | | | | |
| 51 | 139 | MHPQHP | 89% | 7% | 17% | 76% |
| | | (SEQ ID NO: 521) | | | | |
| 15 | 54 | MDHDH | 109% | 8% | 17% | 75% |
| | | (SEQ ID NO: 603) | | | | |
| 46 | 96 | MHSGHD | 104% | 9% | 15% | 75% |
| | | (SEQ ID NO: 651) | | | | |
| 57 | 146 | MHGEHG | 103% | 7% | 18% | 75% |
| | | (SEQ ID NO: 529) | | | | |
| 65 | 168 | MHKEHK | 104% | 8% | 18% | 75% |
| | | (SEQ ID NO: 553) | | | | |
| 61 | 164 | MHAKHA | 89% | 7% | 19% | 74% |
| | | (SEQ ID NO: 548) | | | | |
| 59 | 151 | MHGPHG | 27% | 10% | 16% | 74% |
| | | (SEQ ID NO: 534) | | | | |
| 43 | 93 | MHSNHD | 115% | 10% | 16% | 73% |
| | | (SEQ ID NO: 648) | | | | |
| 44 | 94 | MHSQHD | 99% | 8% | 19% | 73% |
| | | (SEQ ID NO: 649) | | | | |
| 83 | 205 | MHQQHQ | 97% | 8% | 19% | 73% |
| | | (SEQ ID NO: 503) | | | | |
| 52 | 140 | MHPGHP | 95% | 9% | 18% | 73% |
| | | (SEQ ID NO: 522) | | | | |
| 16 | 55 | EHGMGHNT | 100% | 7% | 21% | 72% |
| | | (SEQ ID NO: 606) | | | | |
| 64 | 167 | MHKDHK | 255% | 12% | 16% | 72% |
| | | (SEQ ID NO: 552) | | | | |
| 53 | 141 | MHPAHP | 97% | 11% | 17% | 72% |
| | | (SEQ ID NO: 523) | | | | |
| 17 | 56 | MHDHD | 143% | 13% | 15% | 72% |
| | | (SEQ ID NO: 597) | | | | |
| 39 | 89 | MHEYHD | 111% | 9% | 19% | 72% |
| | | (SEQ ID NO: 644) | | | | |
| 60 | 163 | MHAGHA | 104% | 10% | 18% | 72% |
| | | (SEQ ID NO: 547) | | | | |
| 37 | 78 | MHDYHD | 102% | 11% | 18% | 72% |
| | | (SEQ ID NO: 454) | | | | |
| 45 | 95 | MHSPHD | 107% | 12% | 18% | 70% |
| | | (SEQ ID NO: 650) | | | | |
| 42 | 92 | MHSTHD | 105% | 11% | 19% | 70% |
| | | (SEQ ID NO: 647) | | | | |
| 96 | 223 | HYHYH | 95% | 16% | 15% | 69% |
| | | (SEQ ID NO: 361) | | | | |
| 40 | 90 | MHSDHD | 99% | 12% | 21% | 68% |
| | | (SEQ ID NO: 645) | | | | |
| 30 | 69 | MHDSHD | 98% | 11% | 22% | 67% |
| | | (SEQ ID NO: 445) | | | | |
| 47 | 101 | MHTEHT | 394% | 9% | 24% | 67% |
| | | (SEQ ID NO: 480) | | | | |
| 48 | 134 | MHPDHP | 99% | 11% | 24% | 66% |
| | | (SEQ ID NO: 516) | | | | |
| 41 | 91 | MHSEHD | 106% | 12% | 22% | 65% |
| | | (SEQ ID NO: 646) | | | | |
| 36 | 75 | MHDAHD | 103% | 12% | 23% | 65% |
| | | (SEQ ID NO: 451) | | | | |
| 31 | 70 | MHDTHD | 106% | 12% | 24% | 65% |
| | | (SEQ ID NO: 446) | | | | |
| 35 | 74 | MHDGHD | 94% | 12% | 23% | 65% |
| | | (SEQ ID NO: 450) | | | | |
| 34 | 73 | MHDPHD | 80% | 14% | 22% | 65% |
| | | (SEQ ID NO: 449) | | | | |
| 38 | 84 | MHEPHD | 98% | 14% | 22% | 64% |
| | | (SEQ ID NO: 639) | | | | |
| 33 | 72 | MHDQHD | 104% | 13% | 24% | 63% |
| | | (SEQ ID NO: 448) | | | | |
| 32 | 71 | MHDNHD | 101% | 16% | 25% | 60% |
| | | (SEQ ID NO: 447) | | | | |
| 29 | 68 | MHDEHD | 103% | 13% | 30% | 57% |
| | | (SEQ ID NO: 444) | | | | |
| 78 | 200 | MHDDHD | 98% | 17% | 27% | 56% |
| | | (SEQ ID NO: 443) | | | | |
| 79 | 201 | MHEEHE | 100% | 17% | 29% | 53% |
| | | (SEQ ID NO: 455) | | | | |
| 8 | 47 | HDKG | 101% | 54% | 31% | 15% |
| | | (SEQ ID NO: 669) | | | | |
| 10 | 49 | HDRG | 107% | 52% | 33% | 14% |
| | | (SEQ ID NO: 668) | | | | |
| 7 | 46 | EGHGE | 99% | 66% | 27% | 6% |
| | | (SEQ ID NO: 236) | | | | |

TABLE 25

| % Recov | % FT | % W₂₀ | % E | Peptide |
|---|---|---|---|---|
| 81% | 2% | 2% | 96% | MHQHYQ |
| | | | | (SEQ ID NO: 514) |
| 90% | 2% | 2% | 96% | HEHEH |
| | | | | (SEQ ID NO: 351) |
| 75% | 3% | 2% | 95% | HGAHDHG |
| | | | | (SEQ ID NO: 628) |
| 88% | 2% | 3% | 95% | MHQRHQ |
| | | | | (SEQ ID NO: 513) |
| 93% | 3% | 2% | 95% | MHRSHR |
| | | | | (SEQ ID NO: 566) |
| 93% | 2% | 3% | 95% | MHNRHN |
| | | | | (SEQ ID NO: 501) |
| 108% | 3% | 2% | 95% | MHKRHK |
| | | | | (SEQ ID NO: 561) |
| 109% | 3% | 3% | 95% | MHRRHR |
| | | | | (SEQ ID NO: 563) |
| 97% | 3% | 3% | 95% | GSHDH |
| | | | | (SEQ ID NO: 604) |
| 92% | 2% | 3% | 95% | MHQKHQ |
| | | | | (SEQ ID NO: 512) |
| 103% | 2% | 3% | 94% | HGDGH |
| | | | | (SEQ ID NO: 43) |
| 64% | 3% | 2% | 94% | HRHRH |
| | | | | (SEQ ID NO: 360) |
| 93% | 3% | 3% | 94% | MHNYHN |
| | | | | (SEQ ID NO: 502) |
| 100% | 3% | 3% | 94% | MHQSHQ |
| | | | | (SEQ ID NO: 506) |
| 96% | 3% | 2% | 94% | MHRTHR |
| | | | | (SEQ ID NO: 567) |
| 86% | 4% | 2% | 94% | MHRQHR |
| | | | | (SEQ ID NO: 569) |
| 101% | 3% | 3% | 94% | MHNTHN |
| | | | | (SEQ ID NO: 495) |
| 102% | 3% | 3% | 94% | MHRNHR |
| | | | | (SEQ ID NO: 568) |
| 106% | 3% | 3% | 94% | MHNGHN |
| | | | | (SEQ ID NO: 498) |
| 105% | 4% | 3% | 94% | GSHGAH |
| | | | | (SEQ ID NO: 630) |
| 18% | 4% | 2% | 94% | HHHHHH |
| | | | | (SEQ ID NO: 622) |
| 89% | 4% | 3% | 94% | MHRKHR |
| | | | | (SEQ ID NO: 573) |
| 99% | 4% | 3% | 94% | MHGAHG |
| | | | | (SEQ ID NO: 535) |
| 94% | 3% | 4% | 93% | MHPPHP |
| | | | | (SEQ ID NO: 515) |
| 92% | 3% | 4% | 93% | MHNKHN |
| | | | | (SEQ ID NO: 500) |
| 93% | 3% | 4% | 93% | MHQPHQ |
| | | | | (SEQ ID NO: 509) |
| 99% | 3% | 4% | 93% | MHNQHN |
| | | | | (SEQ ID NO: 496) |
| 101% | 3% | 4% | 93% | MHQGHQ |
| | | | | (SEQ ID NO: 510) |
| 88% | 5% | 2% | 93% | MHRAHR |
| | | | | (SEQ ID NO: 572) |
| 99% | 4% | 3% | 93% | MHRDHR |
| | | | | (SEQ ID NO: 564) |
| 96% | 3% | 4% | 93% | MHTSHS |
| | | | | (SEQ ID NO: 656) |
| 97% | 3% | 4% | 93% | MHQAHQ |
| | | | | (SEQ ID NO: 511) |
| 105% | 3% | 4% | 93% | MHNAHN |
| | | | | (SEQ ID NO: 499) |
| 91% | 3% | 4% | 93% | MHAQHA |
| | | | | (SEQ ID NO: 545) |
| 93% | 4% | 4% | 93% | MHGTHG |
| | | | | (SEQ ID NO: 531) |
| 93% | 4% | 4% | 93% | MHNPHN |
| | | | | (SEQ ID NO: 497) |
| 95% | 4% | 4% | 92% | MHREHR |
| | | | | (SEQ ID NO: 565) |
| 104% | 5% | 3% | 92% | MHRGHR |
| | | | | (SEQ ID NO: 571) |
| 87% | 4% | 4% | 92% | MHGQHG |
| | | | | (SEQ ID NO: 533) |
| 111% | 5% | 3% | 92% | MHKAHK |
| | | | | (SEQ ID NO: 560) |
| 119% | 5% | 3% | 91% | MHKNHK |
| | | | | (SEQ ID NO: 556) |
| 99% | 6% | 3% | 91% | MHKYHK |
| | | | | (SEQ ID NO: 562) |
| 102% | 4% | 5% | 91% | MHNDHN |
| | | | | (SEQ ID NO: 492) |
| 93% | 6% | 3% | 91% | MHRPHR |
| | | | | (SEQ ID NO: 570) |
| 95% | 4% | 5% | 91% | MHQTHQ |
| | | | | (SEQ ID NO: 507) |
| 130% | 5% | 4% | 91% | MSHKHD |
| | | | | (SEQ ID NO: 671) |
| 94% | 4% | 5% | 91% | MHNSHN |
| | | | | (SEQ ID NO: 494) |
| 99% | 4% | 5% | 91% | MHAEHA |
| | | | | (SEQ ID NO: 541) |
| 95% | 6% | 3% | 91% | MHSRHD |
| | | | | (SEQ ID NO: 654) |
| 101% | 6% | 3% | 91% | MHKQHK |
| | | | | (SEQ ID NO: 557) |
| 96% | 4% | 5% | 90% | MHPTHP |
| | | | | (SEQ ID NO: 519) |
| 94% | 5% | 5% | 90% | MHAPHA |
| | | | | (SEQ ID NO: 546) |
| 105% | 4% | 5% | 90% | MHQDHQ |
| | | | | (SEQ ID NO: 504) |
| 102% | 5% | 4% | 90% | MHKPHK |
| | | | | (SEQ ID NO: 558) |
| 97% | 6% | 4% | 90% | MHTNHD |
| | | | | (SEQ ID NO: 658) |
| 79% | 6% | 4% | 90% | MHYQHY |
| | | | | (SEQ ID NO: 581) |
| 104% | 5% | 6% | 90% | MHNEHN |
| | | | | (SEQ ID NO: 493) |
| 100% | 7% | 4% | 89% | MHKGHK |
| | | | | (SEQ ID NO: 559) |
| 119% | 6% | 5% | 89% | MHTRHD |
| | | | | (SEQ ID NO: 664) |
| 105% | 7% | 4% | 89% | MHTPHD |
| | | | | (SEQ ID NO: 660) |
| 114% | 7% | 4% | 89% | MHSAHD |
| | | | | (SEQ ID NO: 652) |
| 80% | 8% | 3% | 89% | MHRYHR |
| | | | | (SEQ ID NO: 574) |
| 105% | 5% | 6% | 89% | MHQEHQ |
| | | | | (SEQ ID NO: 505) |
| 91% | 6% | 5% | 88% | MHQNHQ |
| | | | | (SEQ ID NO: 508) |
| 111% | 8% | 4% | 88% | MHTAHD |
| | | | | (SEQ ID NO: 662) |
| 107% | 7% | 5% | 88% | MHTQHD |
| | | | | (SEQ ID NO: 659) |
| 101% | 7% | 6% | 88% | MHEGHD |
| | | | | (SEQ ID NO: 640) |
| 104% | 6% | 6% | 88% | MHENHD |
| | | | | (SEQ ID NO: 637) |
| 112% | 7% | 6% | 87% | MHDRHD |
| | | | | (SEQ ID NO: 453) |
| 108% | 6% | 6% | 87% | MHERHD |
| | | | | (SEQ ID NO: 643) |
| 111% | 8% | 5% | 87% | MHTGHD |
| | | | | (SEQ ID NO: 661) |
| 105% | 10% | 4% | 86% | MHSYHD |
| | | | | (SEQ ID NO: 655) |
| 124% | 8% | 6% | 86% | MHDKHD |
| | | | | (SEQ ID NO: 452) |
| 117% | 8% | 7% | 86% | MHEKHD |
| | | | | (SEQ ID NO: 642) |
| 99% | 7% | 7% | 85% | MHEQHD |
| | | | | (SEQ ID NO: 638) |
| 97% | 10% | 5% | 85% | MHTKHD |
| | | | | (SEQ ID NO: 663) |
| 98% | 11% | 4% | 85% | GSHDH |
| | | | | (SEQ ID NO: 604) |
| 106% | 8% | 7% | 85% | MHTDHD |
| | | | | (SEQ ID NO: 657) |
| 101% | 9% | 7% | 84% | MHETHD |
| | | | | (SEQ ID NO: 636) |
| 153% | 9% | 9% | 83% | MHPEHP |
| | | | | (SEQ ID NO: 517) |
| 96% | 10% | 8% | 82% | MHATHA |
| | | | | (SEQ ID NO: 543) |
| 110% | 11% | 8% | 81% | MHESHD |
| | | | | (SEQ ID NO: 635) |
| 118% | 11% | 8% | 81% | MHASHA |
| | | | | (SEQ ID NO: 542) |
| 106% | 15% | 5% | 80% | MHTYHD |
| | | | | (SEQ ID NO: 665) |
| 99% | 12% | 8% | 80% | MHANHA |
| | | | | (SEQ ID NO: 544) |
| 109% | 11% | 9% | 80% | MHADHA |
| | | | | (SEQ ID NO: 540) |
| 104% | 11% | 10% | 79% | MHEDHD |
| | | | | (SEQ ID NO: 634) |
| 59% | 16% | 7% | 77% | MHYYHY |
| | | | | (SEQ ID NO: 575) |
| 96% | 15% | 8% | 77% | MHGYHG |
| | | | | (SEQ ID NO: 538) |
| 106% | 18% | 9% | 73% | MHGKHG |
| | | | | (SEQ ID NO: 536) |
| 106% | 19% | 9% | 72% | MHGNHG |
| | | | | (SEQ ID NO: 532) |
| 97% | 21% | 10% | 69% | MHGDHG |
| | | | | (SEQ ID NO: 528) |
| 87% | 19% | 15% | 66% | MHEAHD |
| | | | | (SEQ ID NO: 641) |
| 102% | 28% | 10% | 62% | MHGRHG |
| | | | | (SEQ ID NO: 537) |
| 90% | 95% | 4% | 0% | GGGGGG |
| | | | | (SEQ ID NO: 672) |
| 117% | 96% | 4% | 0% | GAGAGA |
| | | | | (SEQ ID NO: 673) |

For peptides of the general formula R1-H(XᵢH)ⱼ—R2 (SEQ ID NO: 15) wherein i=an integer from 1 to 10, and j=1-10, with the proviso that when j≧2, at least one pair of Xᵢ adjacent to the same histidine do not have the same number of amino acids. Each Xᵢ may independently be from 1 to 10 amino acid residues drawn from the set of the 20 naturally occurring amino acids commonly found in proteins in either the L or D form of chiral amino acids or a modified amino acid with the proviso that the amino acid of X adjacent to H is not histidine. The amino acid in the position of the R1-proximal “X” may be the same or different as the amino acid in the position of the R2-proximal “X”. The R1-proximal “X” may or may not have the same value for “i” as does the R2-proximal “X”. R1 and R2 may independently be hydrogen, one or more amino acids or a protein sequence of interest. Using the analysis described above, various preferred peptide sequences were identified and are presented in the following Tables 26-30 where 3.1=a preferred peptide, 4=a more preferred peptide, and 4.1=a most preferred peptide, N=N-terminal, C=C-terminal, NC=either N- or C-terminal.

TABLE 26
i = 1, j = 1, −NC

| | Preferred | | Preferred |
|---|---|---|---|
| Sequence | status | Sequence | status |
| HGH | 4 | MHEHE | 4 |
| | | (SEQ ID NO: 598) | |
| HEH | 4 | MHSHS | 4 |
| | | (SEQ ID NO: 599) | |
| HDH | 4 | MHTHT | 4 |
| | | (SEQ ID NO: 600) | |
| HDHG | 4 | MHNHN | 4 |
| (SEQ ID NO: 626) | | (SEQ ID NO: 596) | |
| MDHDH | 4 | MHQHQ | 4 |
| (SEQ ID NO: 603) | | (SEQ ID NO: 590) | |
| GSHGH | 4 | MHPHP | 4 |
| (SEQ ID NO: 605) | | (SEQ ID NO: 591) | |
| GSHDH | 4 | MHGHG | 4 |
| (SEQ ID NO: 604) | | (SEQ ID NO: 592) | |
| GSHDHG | 4 | MHAHA | 4 |
| (SEQ ID NO: 631) | | (SEQ ID NO: 593) | |
| SHDHG | 4 | MHKHK | 4 |
| (SEQ ID NO: 623) | | (SEQ ID NO: 594) | |
| MHDHD | 4 | MHRHR | 4 |
| (SEQ ID NO: 597) | | (SEQ ID NO: 595) | |
| | | MHQHYQ | 4 |
| | | (SEQ ID NO: 514) | |

TABLE 27
i = 2, j = 1, −NC

| | Preferred | | Preferred |
|---|---|---|---|
| Sequence | status | Sequence | status |
| MHDTHD | 4.1 | MHSNHD | 4.1 |
| (SEQ ID NO: 446) | | (SEQ ID NO: 648) | |
| MHSTHD | 4.1 | MHDQHD | 4.1 |
| (SEQ ID NO: 647) | | (SEQ ID NO: 448) | |
| MHESHD | 4.1 | MHSQHD | 4.1 |
| (SEQ ID NO: 635) | | (SEQ ID NO: 649) | |
| MHSEHD | 4.1 | MHDPHD | 4.1 |
| (SEQ ID NO: 646) | | (SEQ ID NO: 449) | |
| MHDEHD | 4.1 | MHEPHD | 4.1 |
| (SEQ ID NO: 444) | | (SEQ ID NO: 639) | |
| MHEDHD | 4.1 | MHSPHD | 4.1 |
| (SEQ ID NO: 634) | | (SEQ ID NO: 650) | |
| MHSDHD | 4.1 | MHDGHD | 4.1 |
| (SEQ ID NO: 645) | | (SEQ ID NO: 450) | |
| MHYGHY | 4.1 | MHDAHD | 4.1 |
| (SEQ ID NO: 583) | | (SEQ ID NO: 451) | |
| MHYAHY | 4.1 | MHEAHD | 4.1 |
| (SEQ ID NO: 584) | | (SEQ ID NO: 641) | |
| MHPYHP | 4.1 | MHEKHD | 4.1 |
| (SEQ ID NO: 526) | | (SEQ ID NO: 642) | |
| MHYDHY | 4.1 | MHDYHD | 4.1 |
| (SEQ ID NO: 576) | | (SEQ ID NO: 454) | |
| MHYEHY | 4.1 | | |
| (SEQ ID NO: 577) | | | |
| MHDDHD | 4 | GSHGAH | 4 |
| (SEQ ID NO: 443) | | (SEQ ID NO: 630) | |
| MHSSHS | 4 | MHPPHP | 4 |
| (SEQ ID NO: 467) | | (SEQ ID NO: 515) | |
| MHTTHT | 4 | MHAAHA | 4 |
| (SEQ ID NO: 479) | | (SEQ ID NO: 539) | |
| MHNNHN | 4 | MHRRHR | 4 |
| (SEQ ID NO: 491) | | (SEQ ID NO: 563) | |
| MHQQHQ | 4 | MHADHA | 4 |
| (SEQ ID NO: 503) | | (SEQ ID NO: 540) | |
| MHTEHT | 4 | MHPEHP | 4 |
| (SEQ ID NO: 480) | | (SEQ ID NO: 517) | |
| MHNDHN | 4 | MHRDHR | 4 |
| (SEQ ID NO: 492) | | (SEQ ID NO: 564) | |
| MHQDHQ | 4 | MHAEHA | 4 |
| (SEQ ID NO: 504) | | (SEQ ID NO: 541) | |
| MHTSHS | 4 | MHREHR | 4 |
| (SEQ ID NO: 656) | | (SEQ ID NO: 565) | |
| MHNEHN | 4 | MHPSHP | 4 |
| (SEQ ID NO: 493) | | (SEQ ID NO: 518) | |
| MHQEHQ | 4 | MHGSHG | 4 |
| (SEQ ID NO: 505) | | (SEQ ID NO: 530) | |
| MHETHD | 4 | MHASHA | 4 |
| (SEQ ID NO: 636) | | (SEQ ID NO: 542) | |
| MHNSHN | 4 | MHRSHR | 4 |
| (SEQ ID NO: 494) | | (SEQ ID NO: 566) | |
| MHQSHQ | 4 | MHPTHP | 4 |
| (SEQ ID NO: 506) | | (SEQ ID NO: 519) | |
| MHTNHD | 4 | MHGTHG | 4 |
| (SEQ ID NO: 658) | | (SEQ ID NO: 531) | |
| MHNTHN | 4 | MHRTHR | 4 |
| (SEQ ID NO: 495) | | (SEQ ID NO: 567) | |
| MHQTHQ | 4 | MHANHA | 4 |
| (SEQ ID NO: 507) | | (SEQ ID NO: 544) | |
| MHEQHD | 4 | MHKNHK | 4 |
| (SEQ ID NO: 638) | | (SEQ ID NO: 556) | |
| MHTQHD | 4 | MHRNHR | 4 |
| (SEQ ID NO: 659) | | (SEQ ID NO: 568) | |
| MHNQHN | 4 | MHPQHP | 4 |
| (SEQ ID NO: 496) | | (SEQ ID NO: 521) | |
| MHQNHQ | 4 | MHGQHG | 4 |
| (SEQ ID NO: 508) | | (SEQ ID NO: 533) | |
| MHTPHD | 4 | MHAQHA | 4 |
| (SEQ ID NO: 660) | | (SEQ ID NO: 545) | |
| MHNPHN | 4 | MHKQHK | 4 |
| (SEQ ID NO: 497) | | (SEQ ID NO: 557) | |
| MHQPHQ | 4 | MHRQHR | 4 |
| (SEQ ID NO: 509) | | (SEQ ID NO: 569) | |
| MHEGHD | 4 | MHPGHP | 4 |
| (SEQ ID NO: 640) | | (SEQ ID NO: 522) | |
| MHTGHD | 4 | MHAPHA | 4 |
| (SEQ ID NO: 661) | | (SEQ ID NO: 546) | |
| MHNGHN | 4 | MHKPHK | 4 |
| (SEQ ID NO: 498) | | (SEQ ID NO: 558) | |
| MHQGHQ | 4 | MHRPHR | 4 |
| (SEQ ID NO: 510) | | (SEQ ID NO: 570) | |
| MHSAHD | 4 | MHGAHG | 4 |
| (SEQ ID NO: 652) | | (SEQ ID NO: 535) | |
| MHTAHD | 4 | MHKGHK | 4 |
| (SEQ ID NO: 662) | | (SEQ ID NO: 559) | |
| MHNAHN | 4 | MHRGHR | 4 |
| (SEQ ID NO: 499) | | (SEQ ID NO: 571) | |
| MHQAHQ | 4 | MHPKHP | 4 |
| (SEQ ID NO: 511) | | (SEQ ID NO: 524) | |
| MHTKHD | 4 | MHGKHG | 4 |
| (SEQ ID NO: 663) | | (SEQ ID NO: 536) | |
| MHNKHN | 4 | MHKAHK | 4 |
| (SEQ ID NO: 500) | | (SEQ ID NO: 560) | |
| MHQKHQ | 4 | MHRAHR | 4 |
| (SEQ ID NO: 512) | | (SEQ ID NO: 572) | |
| MHDRHD | 4 | MHPRHP | 4 |
| (SEQ ID NO: 453) | | (SEQ ID NO: 525) | |
| MHERHD | 4 | MHARHA | 4 |
| (SEQ ID NO: 643) | | (SEQ ID NO: 549) | |
| MHSRHD | 4 | MHKRHK | 4 |
| (SEQ ID NO: 654) | | (SEQ ID NO: 561) | |
| MHTRHD | 4 | MHRKHR | 4 |
| (SEQ ID NO: 664) | | (SEQ ID NO: 573) | |
| MHNRHN | 4 | MHAYHA | 4 |
| (SEQ ID NO: 501) | | (SEQ ID NO: 550) | |
| MHQRHQ | 4 | MHKYHK | 4 |
| (SEQ ID NO: 513) | | (SEQ ID NO: 562) | |
| MHNYHN | 4 | | |
| (SEQ ID NO: 502) | | | |
| MHGEHG | 3.1 | MHDSHD | 3.1 |
| (SEQ ID NO: 529) | | (SEQ ID NO: 445) | |
| MHKEHK | 3.1 | MHTDHD | 3.1 |
| (SEQ ID NO: 553) | | (SEQ ID NO: 657) | |
| MHKSHK | 3.1 | MHENHD | 3.1 |
| (SEQ ID NO: 554) | | (SEQ ID NO: 637) | |
| MHYSHY | 3.1 | MHSGHD | 3.1 |
| (SEQ ID NO: 578) | | (SEQ ID NO: 651) | |
| MHKTHK | 3.1 | MHDKHD | 3.1 |
| (SEQ ID NO: 555) | | (SEQ ID NO: 452) | |
| MHYTHY | 3.1 | MHSKHD | 3.1 |
| (SEQ ID NO: 579) | | (SEQ ID NO: 653) | |
| MHPNHP | 3.1 | MHEYHD | 3.1 |
| (SEQ ID NO: 520) | | (SEQ ID NO: 644) | |
| MHYNHY | 3.1 | MHTYHD | 3.1 |
| (SEQ ID NO: 580) | | (SEQ ID NO: 665) | |
| MHYPHY | 3.1 | MHKDHK | 3.1 |
| (SEQ ID NO: 582) | | (SEQ ID NO: 552) | |

TABLE 28
i = 3, j = 1, −NC

| | Preferred |
|---|---|
| Sequence | status |
| HGDGH (SEQ ID NO: 43) | 4 |
| HGEGH (SEQ ID NO: 52) | 4 |
| HSDGH (SEQ ID NO: 196) | 4 |
| HGESH (SEQ ID NO: 178) | 4 |
| EHGMGHNT (SEQ ID NO: 606) | 3.1 |

TABLE 29
i = 4, j = 1, −NC

| Sequence | Preferred status |
|---|---|
| HGARGH (SEQ ID NO: 602) | 4 |
| HGAKGH (SEQ ID NO: 601) | 4 |

TABLE 30
i = 1, j = 2, −C

| Sequence | Preferred status |
|---|---|
| HDHDH (SEQ ID NO: 350) | 4 |
| HEHEH (SEQ ID NO: 351) | 4 |
| HGHGH (SEQ ID NO: 357) | 4 |
| HSHSH (SEQ ID NO: 352) | 4 |
| HAHAH (SEQ ID NO: 358) | 4 |
| HTHTH (SEQ ID NO: 353) | 4 |
| HKHKH (SEQ ID NO: 359) | 4 |
| HNHNH (SEQ ID NO: 354) | 4 |
| HRHRH(SEQ ID NO: 360) | 4 |
| HQHQH (SEQ ID NO: 355) | 3.1 |
| HYHYH (SEQ ID NO: 361) | 3.1 |
| HPHPH (SEQ ID NO: 356) | 3.1 |
| HMHMH (SEQ ID NO: 362) | 3.1 |

TABLE 31
i = 1 and i = 2, j = 2, −C

| Sequence | Preferred status |
|---|---|
| HGAHDHG (SEQ ID NO: 628) | 4 |
| HGAHGH (SEQ ID NO: 363) | 4 |
| HVHGAH (SEQ ID NO: 666) | 4 |
| HDDHDH (SEQ ID NO: 367) | 4 |
| HDHDDH (SEQ ID NO: 667) | 4 |

### Example 4

Binding of recombinant fusion proteins to nickel matrices

Nucleic acid molecules encoding fusion proteins comprising a peptide of the invention and an additional protein sequence were constructed and tested for binding to immobilized metal ions. The additional sequence was the chloramphenicol acetyl transferase gene (CAT).

The following buffers were used:
Buffer A: 25 mM NaPO₄, pH 7.5, 5% glycerol, 500 mM NaCl, 5 mM imidazole
Buffer B (wash 1): Buffer A with 10 mM imidazole
Buffer C (wash 2): Buffer A with 20 mM imidazole
Buffer D (elution 1): Buffer A with 50 mM imidazole
Buffer E (elution 2): Buffer A with 500 mM imidazole

Extracts were prepared as follows
1) overnight cultures of host cells comprising a nucleic acid molecule encoding a fusion protein of the invention were diluted 1:50 into 25 mls LB-Ap100-Cm10 and grown to OD 0.6;
2) samples were induced with 1 mM IPTG and grown for 2 hours;
3) cells were harvested, frozen at −80, thawed, and resuspended in 0.5 mls buffer A; and
4) resuspended samples were sonicated 3×15 seconds with a microtip and centrifuged to produce clarified extracts.

Clarified extracts were applied to Qiagen NiNTA Spin Columns as follows:
1) A NiNTA spin column was equilibrated with 600 μl Buffer A and centrifuged 2 min at 700 g
2) 400 μl extract was loaded onto the column, and the flowthrough was reloaded a second time;
3) 600 μl Buffer A was used to wash the column twice;
4) 600 μl Buffer C was used to wash the column once;
5) 400 μl Buffer D was used to wash the column once; and
6) 200 μl Buffer E was used to elute the column twice.

Extracts may also be analyzed using Pierce SwellGel Beads as follows:
1) Place a Swellgel bead into a mini-spin column (Biorad)—leave end cap sealed;
2) Add 400 μl extract to the column, incubate RT 5 minutes;
3) Vortex briefly, break off end cap, spin 2 min at 1,000 rpm (collect as FT);
4) Add 400 μl Buffer A, vortex briefly, spin 2 min at 1,000 rpm, repeat once;
5) Wash twice as above with 400 μl Buffer C (collect as W and W2);
6) Add 200 μl Buffer E and let sit for 5 minutes at RT;
7) Vortex briefly, spin 2 min at 1,000 rpm; and
8) Add 200 μl Buffer E, vortex briefly, spin 2 min into same tube (collect as E).

The binding characteristics of the fusion proteins were analyzed using SDS-PAGE. Samples were loaded onto Novex NuPAGE gels for analysis. Generally, 2 μl of loads and FT fractions, and 10 μl of wash and eluant fractions were loaded.

Fusion proteins comprising the following peptides of the invention were prepared and analyzed with the following results.
1) SlyDC1 (amino acids 149-196) (SEQ ID NO: 608)—C-terminal tag on CAT; all protein bound;
2) SlyDC2 (amino acids 149-165) (SEQ ID NO: 609)—C-terminal tag on CAT; all protein bound;
3) SlyDC3 (amino acids 151-160) (SEQ ID NO: 610)—C-terminal tag on CAT; all protein bound;
4) SlyDC4 (amino acids 151-160, H159G) (SEQ ID NO: 611)—C-terminal tag on CAT; all protein bound, some elution at 50 mM imidazole;
5) SlyDC5 (amino acids 151-157) (SEQ ID NO: 612)—C-terminal tag on CAT; >90% protein bound, 35% elution at 20 mM, 34% at 50 mM imidazole;
6) SlyDC6 (amino acids 156-159, H159G) (SEQ ID NO: 613)—C-terminal tag on CAT; >95% bound, 40% elution at 20 mM, 30% at 50 mM imidazole;
7) SlyDC7 (amino acids 153-159, H159G) (SEQ ID NO: 614)—C-terminal tag on CAT; >95% bound, 28% elution at 20 mM, 35% at 50 mM imidazole.

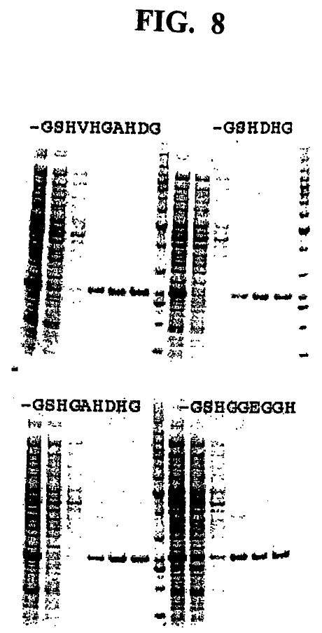

FIG. 8 provides a representative gel analysis of the binding characteristics of the indicated peptides illustrating elution of the fusion protein upon addition of imidazole, as shown in lanes 4-6 of each series.

### Example 5

### Peptide with Bifunctional Utility

The peptide FITC-EHGMGHNT (SEQ ID NO: 674) represents the conserved intein motif known as “Block G.” It was chemically synthesized and tested in a nickel binding assay as in Ex. 2. As shown in FIG. 7 (left column, second group, fourth peptide), this peptide exhibits favorable binding and elution characteristics. Thus, this peptide has potential utility as a bifunctional fusion tag as it can function as both a purification tag and as an intein site.

### Example 6

### Analysis of Fusion Proteins with Specific Peptide Sequences

This Example provides an analysis of binding of a number of histidine-rich peptides of the present invention, associated with a polypeptide as part of a fusion protein. The peptides were designed to bind to metal chelate affinity chromatography media when one or more metal ions are bound to these media. The peptides that were tested had sequences according to the following sequence patterns:
1. HxHxHxHxHxHx (SEQ ID NO: 675)
2. HxHxx HxHxx HxHxx (SEQ ID NO: 676)

The following DNA sequences were used as 5′-forward PCR primers to amplify the gene for Mja (LOCUS Q58559; 645 amino acids; Replication factor A (RP-A) (RF-A) (Replication factor-A protein 1) (Single-stranded DNA-binding protein) (mjaSSB). ACCESSION Q58559; GI:46577162) with the histidine-rich peptides added onto the amino terminus of the Mja protein. The first 7 bases are a header, the 2^{nd} 6 bases are Met-Gly followed by the respective histidine-rich sequences. The final 20 bases are homologous to the Mja sequence.

(SEQ ID NO: 677)

| | |
|---|---|
| 1 | aggttcc atggga cactcgcattcacacagccactctcacagcca |
| | ttcc ggagtaggagattatgaaag |

(SEQ ID NO: 678)

| | |
|---|---|
| 2 | aggttcc atggga cactcgcattcaagtcacagccactcttcaca |
| | cagccat ggagtaggagattatgaaag |

(SEQ ID NO: 679)

| | |
|---|---|
| 3 | aggttcc atggga cactcgcattcaagtcacagccactcttcgca |
| | cagccattccagtcacagccacggagtaggagattatgaaag |

(SEQ ID NO: 680)

| | |
|---|---|
| 4 | aggttcc atggga cacaaacataagcacaagcacaaacacaagca |
| | c ggagtaggagattatgaaag |

(SEQ ID NO: 681)

| | |
|---|---|
| 5 | aggttcc atggga cacaaacataagaagcacaagcacaagaaaca |
| | caagcat ggagtaggagattatgaaag |

(SEQ ID NO: 682)

| | |
|---|---|
| 6 | aggttcc atggga cactcacattcaagccactatcataagaaaca |
| | taagcac ggagtaggagattatgaaag |

(SEQ ID NO: 683)

| | |
|---|---|
| 7 | aggttcc atggga cactatcataagaaacataagcactcgagtca |
| | tagccac ggagtaggagattatgaaag |

(SEQ ID NO: 684)

| | |
|---|---|
| 8 | aggttcc atggga cactcacataagagccactatcataagaaaca |
| | taagcactacagtcatagccac ggagtaggagattatgaaag |

(SEQ ID NO: 685)

| | |
|---|---|
| 9 | aggttcc atggga cactcacataagagccactatcattcctcgca |
| | taagcac ggagtaggagattatgaaag |

(SEQ ID NO: 686)

| | |
|---|---|
| 10 | aggttcc atggga cactcacataagagccactatcataagtcgc |
| | attctcac ggagtaggagattatgaaag |

As a result of translation of the above-amplified sequences, the following peptide sequences were added to Mja single stranded DNA binding (SSB) protein in the encoded fusion protein (please note: a methionine residue and a glycine residue were also added to the front of the peptide sequence and a glycine residue was added between the peptide sequence and the start of the Mja sequence):

| | | |
|---|---|---|
| 1 | HSHSHSHSHSHS | (SEQ ID NO: 33) |
| 2 | HSHSSHSHSSHSH | (SEQ ID NO: 5) |
| 3 | HSHSSHSHSSHSHSSHSH | (SEQ ID NO: 3) |
| 4 | HKHKHKHKHKH | (SEQ ID NO: 31) |
| 5 | HKHKKHKHKKHKH | (SEQ ID NO:6) |
| 6 | HSHSSHYHKKHKH | (SEQ ID NO: 7) |
| 7 | HYHKKHKHSSHSH | (SEQ ID NO: 8) |
| 8 | HSHKSHYHKKHKHYSHSH | (SEQ ID NO: 4) |
| 9 | HSHKSHYHSSHKH | (SEQ ID NO: 9) |
| 10 | HSHKSHYHKSHSH | (SEQ ID NO: 10) |

The amino terminal histidine-rich peptides were cloned into Mja first and tested for their ability to bind to Ni²⁺ columns during purification of the recombinant fusion protein.

The following amino terminal histidine-rich peptides (also referred to herein as amino His tags) were tested for binding to 1 mL Ni²⁺ chelated sepharose columns for the FPLC (Amersham) purifier:

| Amino His Tag | mM Imidazole (required for elution) |
|---|---|
| 1 | 60 |
| 3 | 75 |
| 5 | 75 |
| 6 | 60 |
| 8 | 68 |
| 9 | 56 |

Cell paste was resuspended in loading buffer (50 mM Tris HCl pH 8.5, 10 mM immidazole, 5 mM B-mercaptoethanol)+PMSF at a ratio of 2 ml buffer per 1 gram cells. Cells were lysed by sonication, then heated to 80 degrees Celsius for 15 minutes, followed by centrifugation at 16K for 30 minutes in a SS34 rotor. Supernatant was loaded onto a Ni²⁺ chelating column.

All the amino terminal histidine tags bound to the Ni²⁺ column. The two strongest binding tags were of the format: HxHxxHxHxxHxHxx (SEQ ID NO: 1), where x is either a Serine or a Lysine. His-tagged Mja proteins 3, 5 and 8 where dialyzed and rebound to the 1 mL Ni²⁺ column and subjected to multiple stringent washes to eliminate any DNA bound to the SSB protein. These washes included:
1. Ni²⁺ chelating column loading buffer+4.0 M NaCl
2. Ni²⁺ chelating column loading buffer+2.5 M NaCl+40% ethylene glycol.

The resulting eluted protein had no contaminating DNA when examined by agarose gel electrophoresis. Although all of the 3 peptides tested under stringent washing conditions remained at least partially bound to the column, the peptide HSHKSHYHKKHKHYSHSH (SEQ ID NO: 4) overall had the best expression and bound tightest under the high salt and ethylene glycol washes.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1-55. (canceled)

56. An isolated peptide, wherein the peptide is

| | |
|---|---|
| HSHSSHSHSSHSHSSHSH, | (SEQ ID NO: 3) |
| HKHKKHKHKKHKH, | (SEQ ID NO: 6) |
| HSHSSHYHKKHKH, | (SEQ ID NO: 7) |
| HSHKSHYHKKHKHYSHSH, | (SEQ ID NO: 4) |
| HSHKSHYHSSHKH, | (SEQ ID NO: 9) |
| HKHKKHYH, | (SEQ ID NO: 18) |
| HKHKYHKH, | (SEQ ID NO: 687) |
| HKHYKHKH, | (SEQ ID NO: 20) |
| HYHKKHKH, | (SEQ ID NO: 24) |
| HKHKYHYH, | (SEQ ID NO: 19) |
| HKHYKHYH, | (SEQ ID NO: 21) |
| HYHKKHYH, | (SEQ ID NO: 25) |
| HKHYYHKH, | (SEQ ID NO: 22) |
| HYHKYHKH, | (SEQ ID NO: 26) |
| HYHYKHKH, | (SEQ ID NO: 28) |
| HKHYYHYH, | (SEQ ID NO: 23) |
| HYHKYHYH, | (SEQ ID NO: 27) |
| HYHYKHYH, | (SEQ ID NO: 29) |
| or | |
| HYHYYHKH. | (SEQ ID NO: 30) |

57. A fusion protein comprising one or more copies of the peptide of claim 95 and a protein of interest.

58. The fusion protein of claim 57, wherein the protein of interest is selected from a group consisting of enzymes, cytokines, intraceullar signaling peptides, receptors, antibodies, vaccine components, structural proteins, functional proteins, antigenic proteins, epitopes, pathologic proteins, and synthetic peptides.

59. The fusion protein of claim 57, wherein the protein of interest is selected from the group consisting of kinases, peptidases, proteinases, oxidoreductases, nucleases, recombinases, ligases, lyases, isomerases, DNA polymerases, RNA polymerases, reverse transcriptases, topoisomerases, gyrases, helicases, proteic ribosomal subunits, cell cycle check point proteins, furrinases, cytotoxins, prions, transferases, ATPases, and GTPases.

60. The fusion protein of claim 57, further comprising a protease cleavage site between at least one peptide and the protein of interest.

61. The fusion protein of claim 60, wherein the protease cleavage site is selected from a group consisting Factor Xa cleavage site, thrombin cleavage site, TEV NIa protease cleavage site, a caspase cleavage site, Staphylococcus aureus V8 protease cleavage site, enterokinase cleavage site, trypsin cleavage site, chymotrypsin cleavage site, Genenase I cleavage site, and furin cleavage site.

62. The fusion protein of claim 57, further comprising an intein splicing motif capable of facilitating cis or trans splicing.

63. A composition comprising the fusion protein of any one of claims 57-62 and an immobilized metal ion affinity chromatography matrix.

64. The composition of claim 63, wherein the immobilized metal ion is a nickel ion.

65. A nucleic acid molecule encoding the fusion protein according to any one of claims 57-62.

66. A host cell comprising the nucleic acid molecule of claim 65.

67-86. (canceled)

87. A kit for separating a molecule from a mixture, comprising:
(i) a nucleic acid molecule encoding one or more peptides according to claim 95; and
(ii) a resin with immobilized metal ions.

88. The kit of claim 87, wherein said nucleic acid molecule is a vector.

89. The kit of claim 88, wherein said vector comprises one or more promoters.

90. The kit of claim 89, wherein the promoter is a promoter that functions in prokaryotic or eukaryotic cells.

91. The kit of claim 89, wherein the promoter is selected from the group consisting of an SP6 promoter, a CMV promoter, an SV40 promoter, a bacteriophage promoter, a bacteriophage T7 gene 10 promoter, and a host cell native promoter.

92. The kit of claim 87, further comprising one or more buffers.

93. The kit of claim 87, further comprising one or more recombination proteins.

94. The kit of claim 87, further comprising one or more topoisomerase enzymes.

95. A peptide consisting essentially of the formula HxHxxHxHxxHxHxx (SEQ ID NO: 1), wherein x is an amino acid.

96. The peptide of claim 95, wherein x is a naturally occurring amino acid.

97. The peptide of claim 96, wherein at least one x residue is lysine, serine or threonine.

98. The peptide of claim 97, wherein each x independently is lysine, serine, threonine, or tyrosine.

99. The peptide of claim 97, wherein the peptide is
HxHxxHxHxxHxHxxHxH. (SEQ ID NO: 2)

100. The peptide of claim 99, wherein the peptide is

| | |
|---|---|
| HSHSSHSHSSHSHSSHSH | (SEQ ID NO: 3) |
| or | |
| HSHKSHYHKKHKHYSHSH. | (SEQ ID NO: 4) |

101. The peptide of claim 100, wherein the peptide is
HSHKSHYHKKHKHYSHSH. (SEQ ID NO: 4)

102. The peptide of claim 97, wherein the peptide is

| | |
|---|---|
| HSHSSHSHSSHSH, | (SEQ ID NO: 5) |
| HKHKKHKHKKHKH, | (SEQ ID NO: 6) |
| HSHSSHYHKKHKH, | (SEQ ID NO: 7) |
| HYHKKHKHSSHSH, | (SEQ ID NO: 8) |
| HSHKSHYHSSHKH, | (SEQ ID NO: 9) |
| or | |
| HSHKSHYHKSHSH. | (SEQ ID NO: 10) |


SEQUENCE LISTING (687 sequences)

SEQ ID NO: 1 - protein, 15 aa, Artificial Sequence
  Peptide that binds to affinity chromatography media
HXHXXHXHXXHXHXX

SEQ ID NO: 2 - protein, 18 aa, Artificial Sequence
  Peptide that binds to affinity chromatography media
HXHXXHXHXXHXHXXHXH

SEQ ID NO: 3 - protein, 18 aa, Artificial Sequence
  Peptide that binds to affinity chromatography media
HSHSSHSHSSHSHSSHSH

SEQ ID NO: 4 - protein, 18 aa, Artificial Sequence
  Peptide that binds to affinity chromatography media
HSHKSHYHKKHKHYSHSH

SEQ ID NO: 5 - protein, 13 aa, Artificial Sequence
  Peptide that binds to affinity chromatography media
HSHSSHSHSSHSH

SEQ ID NO: 6 - protein, 13 aa, Artificial Sequence
  Peptide that binds to affinity chromatography media
HKHKKHKHKKHKH

SEQ ID NO: 7 - protein, 13 aa, Artificial Sequence
  Peptide that binds to affinity chromatography media
HSHSSHYHKKHKH

SEQ ID NO: 8 - protein, 13 aa, Artificial Sequence
  Peptide that binds to affinity chromatography media
HYHKKHKHSSHSH

SEQ ID NO: 9 - protein, 13 aa, Artificial Sequence
  Peptide that binds to affinity chromatography media
HSHKSHYHSSHKH

SEQ ID NO: 10 - protein, 13 aa, Artificial Sequence
  Peptide that binds to affinity chromatography media
HSHKSHYHKSHSH

SEQ ID NO: 11 - protein, 43 aa, Artificial Sequence
  Synthetic
XXXXXXXXXXXXXXXXXXXXXXXXXXXXXXXXXXXXXXXXXXX

SEQ ID NO: 12 - protein, 43 aa, Artificial Sequence; identical to SEQ ID NO: 11
  Synthetic

SEQ ID NO: 13 - protein, 43 aa, Artificial Sequence
  Synthetic
HXXXXXXHXXXXXXHXXXXXXHXXXXXXHXXXXXXHXXXXXXH

SEQ ID NO: 14 - protein, 5 aa, Artificial Sequence
  Synthetic
XHXHX

SEQ ID NO: 15 - protein, 113 aa, Artificial Sequence
  Synthetic
XHXXXXXXXXXXHXXXXXXXXXXHXXXXXXXXXXHXXXXXXXXXXHXXXXXXXXXXHXXXXXXXXXXHXXXXXXXXXXHXXXXXXXXXXHXXXXXXXXXXHXXXXXXXXXXHX

SEQ ID NO: 16 - protein, 8 aa, Artificial Sequence
  Synthetic
HXHXXHXH

SEQ ID NO: 17 - protein, 12 aa, Artificial Sequence
  Synthetic
HKHKKHKHKKHK

SEQ ID NO: 18 - protein, 8 aa, Artificial Sequence
  Synthetic
HKHKKHYH

SEQ ID NO: 19 - protein, 8 aa, Artificial Sequence
  Synthetic
HKHKYHYH

SEQ ID NO: 20 - protein, 8 aa, Artificial Sequence
  Synthetic
HKHYKHKH

SEQ ID NO: 21 - protein, 8 aa, Artificial Sequence
  Synthetic
HKHYKHYH

SEQ ID NO: 22 - protein, 8 aa, Artificial Sequence
  Synthetic
HKHYYHKH

SEQ ID NO: 23 - protein, 8 aa, Artificial Sequence
  Synthetic
HKHYYHYH

SEQ ID NO: 24 - protein, 8 aa, Artificial Sequence
  Synthetic
HYHKKHKH

SEQ ID NO: 25 - protein, 8 aa, Artificial Sequence
  Synthetic
HYHKKHYH

SEQ ID NO: 26 - protein, 8 aa, Artificial Sequence
  Synthetic
HYHKYHKH

SEQ ID NO: 27 - protein, 8 aa, Artificial Sequence
  Synthetic
HYHKYHYH

SEQ ID NO: 28 - protein, 8 aa, Artificial Sequence
  Synthetic
HYHYKHKH

SEQ ID NO: 29 - protein, 8 aa, Artificial Sequence
  Synthetic
HYHYKHYH

SEQ ID NO: 30 - protein, 8 aa, Artificial Sequence
  Synthetic
HYHYYHKH

SEQ ID NO: 31 - protein, 11 aa, Artificial Sequence
  Synthetic
HKHKHKHKHKH

SEQ ID NO: 32 - protein, 12 aa, Artificial Sequence
  Synthetic
HSHSHSHSHSHG

SEQ ID NO: 33 - protein, 12 aa, Artificial Sequence
  Synthetic
HSHSHSHSHSHS

SEQ ID NO: 34 - protein, 43 aa, Artificial Sequence
  Synthetic
HXXXXXXXXXXXXXXXXXXXXXXXXXXXXXXXXXXXXXXXXXH

SEQ ID NO: 35 - protein, 43 aa, Artificial Sequence
  Synthetic
HXXXXXXXXXXXXXXXXXXXXXXXXXXXXXXXXXXXXXXXXXK

SEQ ID NO: 36 - protein, 43 aa, Artificial Sequence
  Synthetic
HXXXXXXXXXXXXXXXXXXXXXXXXXXXXXXXXXXXXXXXXXR

SEQ ID NO: 37 - protein, 43 aa, Artificial Sequence
  Synthetic
KXXXXXXXXXXXXXXXXXXXXXXXXXXXXXXXXXXXXXXXXXK

SEQ ID NO: 38 - protein, 43 aa, Artificial Sequence
  Synthetic
KXXXXXXXXXXXXXXXXXXXXXXXXXXXXXXXXXXXXXXXXXH

SEQ ID NO: 39 - protein, 43 aa, Artificial Sequence
  Synthetic
KXXXXXXXXXXXXXXXXXXXXXXXXXXXXXXXXXXXXXXXXXR

SEQ ID NO: 40 - protein, 43 aa, Artificial Sequence
  Synthetic
RXXXXXXXXXXXXXXXXXXXXXXXXXXXXXXXXXXXXXXXXXR

SEQ ID NO: 41 - protein, 43 aa, Artificial Sequence
  Synthetic
RXXXXXXXXXXXXXXXXXXXXXXXXXXXXXXXXXXXXXXXXXH

SEQ ID NO: 42 - protein, 43 aa, Artificial Sequence
  Synthetic
RXXXXXXXXXXXXXXXXXXXXXXXXXXXXXXXXXXXXXXXXXK

SEQ ID NO: 43 - protein, 5 aa, Artificial Sequence
  Synthetic
HGDGH

SEQ ID NO: 44 - protein, 5 aa, Artificial Sequence
  Synthetic
HGDGK

SEQ ID NO: 45 - protein, 5 aa, Synthetic
  Synthetic
HGDGR

SEQ ID NO: 46 - protein, 5 aa, Artificial Sequence
  Synthetic
KGDGK

SEQ ID NO: 47 - protein, 5 aa, Artificial Sequence
  Synthetic
KGDGH

SEQ ID NO: 48 - protein, 5 aa, Artificial Sequence
  Synthetic
KGDGR

SEQ ID NO: 49 - protein, 5 aa, Artificial Sequence
  Synthetic
RGDGR

SEQ ID NO: 50 - protein, 5 aa, Artificial Sequence
  Synthetic
RGDGH

SEQ ID NO: 51 - protein, 5 aa, Artificial Sequence
  Synthetic
RGDGK

SEQ ID NO: 52 - protein, 5 aa, Artificial Sequence
  Synthetic
HGEGH

SEQ ID NO: 53 - protein, 5 aa, Artificial Sequence
  Synthetic
HGEGK

SEQ ID NO: 54 - protein, 5 aa, Artificial Sequence
  Synthetic
HGEGR

SEQ ID NO: 55 - protein, 5 aa, Artificial Sequence
  Synthetic
KGEGK

SEQ ID NO: 56 - protein, 5 aa, Artificial Sequence
  Synthetic
KGEGH

SEQ ID NO: 57 - protein, 5 aa, Artificial Sequence
  Synthetic
KGEGR

SEQ ID NO: 58 - protein, 5 aa, Artificial Sequence
  Synthetic
RGEGR

SEQ ID NO: 59 - protein, 5 aa, Artificial Sequence
  Synthetic
RGEGH

SEQ ID NO: 60 - protein, 5 aa, Artificial Sequence
  Synthetic
RGEGK

SEQ ID NO: 61 - protein, 5 aa, Artificial Sequence
  Synthetic
HGCGH

SEQ ID NO: 62 - protein, 5 aa, Artificial Sequence
  Synthetic
HGCGK

SEQ ID NO: 63 - protein, 5 aa, Artificial Sequence
  Synthetic
HGCGR

SEQ ID NO: 64 - protein, 5 aa, Artificial Sequence
  Synthetic
KGCGK

SEQ ID NO: 65 - protein, 5 aa, Artificial Sequence
  Synthetic
KGCGH

SEQ ID NO: 66 - protein, 5 aa, Artificial Sequence
  Synthetic
KGCGR

SEQ ID NO: 67 - protein, 5 aa, Artificial Sequence
  Synthetic
RGCGR

SEQ ID NO: 68 - protein, 5 aa, Artificial Sequence
  Synthetic
RGCGH

SEQ ID NO: 69 - protein, 5 aa, Artificial Sequence
  Synthetic
RGCGK

SEQ ID NO: 70 - protein, 5 aa, Artificial Sequence
  Synthetic
HGMGH

SEQ ID NO: 71 - protein, 5 aa, Artificial Sequence
  Synthetic
HGMGK

SEQ ID NO: 72 - protein, 5 aa, Artificial Sequence
  Synthetic
HGMGR

SEQ ID NO: 73 - protein, 5 aa, Artificial Sequence
  Synthetic
KGMGK

SEQ ID NO: 74 - protein, 5 aa, Artificial Sequence
  Synthetic
KGMGH

SEQ ID NO: 75 - protein, 5 aa, Artificial Sequence
  Synthetic
KGMGR

SEQ ID NO: 76 - protein, 5 aa, Artificial Sequence
  Synthetic
RGMGR

SEQ ID NO: 77 - protein, 5 aa, Artificial Sequence
  Synthetic
RGMGH

SEQ ID NO: 78 - protein, 5 aa, Artificial Sequence
  Synthetic
RGMGK

SEQ ID NO: 79 - protein, 43 aa, Artificial Sequence; identical to SEQ ID NO: 34
  Synthetic

SEQ ID NO: 80 - protein, 43 aa, Artificial Sequence; identical to SEQ ID NO: 35
  Synthetic

SEQ ID NO: 81 - protein, 43 aa, Artificial Sequence; identical to SEQ ID NO: 36
  Synthetic

SEQ ID NO: 82 - protein, 43 aa, Artificial Sequence; identical to SEQ ID NO: 37
  Synthetic

SEQ ID NO: 83 - protein, 43 aa, Artificial Sequence; identical to SEQ ID NO: 38
  Synthetic

SEQ ID NO: 84 - protein, 43 aa, Artificial Sequence; identical to SEQ ID NO: 39
  Synthetic

SEQ ID NO: 85 - protein, 43 aa, Artificial Sequence; identical to SEQ ID NO: 40
  Synthetic

SEQ ID NO: 86 - protein, 43 aa, Artificial Sequence; identical to SEQ ID NO: 41
  Synthetic

SEQ ID NO: 87 - protein, 43 aa, Artificial Sequence; identical to SEQ ID NO: 42
  Synthetic

SEQ ID NO: 88 - protein, 7 aa, Artificial Sequence
  Synthetic
HGGDGGH

SEQ ID NO: 89 - protein, 7 aa, Artificial Sequence
  Synthetic
HGGDGGK

SEQ ID NO: 90 - protein, 7 aa, Artificial Sequence
  Synthetic
HGGDGGR

SEQ ID NO: 91 - protein, 7 aa, Artificial Sequence
  Synthetic
KGGDGGK

SEQ ID NO: 92 - protein, 7 aa, Artificial Sequence
  Synthetic
KGGDGGH

SEQ ID NO: 93 - protein, 7 aa, Artificial Sequence
  Synthetic
KGGDGGR

SEQ ID NO: 94 - protein, 7 aa, Artificial Sequence
  Synthetic
RGGDGGR

SEQ ID NO: 95 - protein, 7 aa, Artificial Sequence
  Synthetic
RGGDGGH

SEQ ID NO: 96 - protein, 7 aa, Artificial Sequence
  Synthetic
RGGDGGK

SEQ ID NO: 97 - protein, 7 aa, Artificial Sequence
  Synthetic
HGGEGGH

SEQ ID NO: 98 - protein, 7 aa, Artificial Sequence
  Synthetic
HGGEGGK

SEQ ID NO: 99 - protein, 7 aa, Artificial Sequence
  Synthetic
HGGEGGR

SEQ ID NO: 100 - protein, 7 aa, Artificial Sequence
  Synthetic
KGGEGGK

SEQ ID NO: 101 - protein, 7 aa, Artificial Sequence
  Synthetic
KGGEGGH

SEQ ID NO: 102 - protein, 7 aa, Artificial Sequence
  Synthetic
KGGEGGR

SEQ ID NO: 103 - protein, 7 aa, Artificial Sequence
  Synthetic
RGGEGGR

SEQ ID NO: 104 - protein, 7 aa, Artificial Sequence
  Synthetic
RGGEGGH

SEQ ID NO: 105 - protein, 7 aa, Artificial Sequence
  Synthetic
RGGEGGK

SEQ ID NO: 106 - protein, 7 aa, Artificial Sequence
  Synthetic
HGGCGGH

SEQ ID NO: 107 - protein, 7 aa, Artificial Sequence
  Synthetic
HGGCGGK

SEQ ID NO: 108 - protein, 7 aa, Artificial Sequence
  Synthetic
HGGCGGR

SEQ ID NO: 109 - protein, 7 aa, Artificial Sequence
  Synthetic
KGGCGGK

SEQ ID NO: 110 - protein, 7 aa, Artificial Sequence
  Synthetic
KGGCGGH

SEQ ID NO: 111 - protein, 7 aa, Artificial Sequence
  Synthetic
KGGCGGR

SEQ ID NO: 112 - protein, 7 aa, Artificial Sequence
  Synthetic
RGGCGGR

SEQ ID NO: 113 - protein, 7 aa, Artificial Sequence
  Synthetic
RGGCGGH

SEQ ID NO: 114 - protein, 7 aa, Artificial Sequence
  Synthetic
RGGCGGK

SEQ ID NO: 115 - protein, 7 aa, Artificial Sequence
  Synthetic
HGSDGSH

SEQ ID NO: 116 - protein, 7 aa, Artificial Sequence
  Synthetic
HGSDGSK

SEQ ID NO: 117 - protein, 7 aa, Artificial Sequence
  Synthetic
HGSDGSR

SEQ ID NO: 118 - protein, 7 aa, Artificial Sequence
  Synthetic
KGSDGSK

SEQ ID NO: 119 - protein, 7 aa, Artificial Sequence
  Synthetic
KGSDGSH

SEQ ID NO: 120 - protein, 7 aa, Artificial Sequence
  Synthetic
KGSDGSR

SEQ ID NO: 121 - protein, 7 aa, Artificial Sequence
  Synthetic
RGSDGSR

SEQ ID NO: 122 - protein, 7 aa, Artificial Sequence
  Synthetic
RGSDGSH

SEQ ID NO: 123 - protein, 7 aa, Artificial Sequence
  Synthetic
RGSDGSK

SEQ ID NO: 124 - protein, 7 aa, Artificial Sequence
  Synthetic
HGSEGSH

SEQ ID NO: 125 - protein, 7 aa, Artificial Sequence
  Synthetic
HGSEGSK

SEQ ID NO: 126 - protein, 7 aa, Artificial Sequence
  Synthetic
HGSEGSR

SEQ ID NO: 127 - protein, 7 aa, Artificial Sequence
  Synthetic
KGSEGSK

SEQ ID NO: 128 - protein, 7 aa, Artificial Sequence
  Synthetic
KGSEGSH

SEQ ID NO: 129 - protein, 7 aa, Artificial Sequence
  Synthetic
KGSEGSR

SEQ ID NO: 130 - protein, 7 aa, Artificial Sequence
  Synthetic
RGSEGSR

SEQ ID NO: 131 - protein, 7 aa, Artificial Sequence
  Synthetic
RGSEGSH

SEQ ID NO: 132 - protein, 7 aa, Artificial Sequence
  Synthetic
RGSEGSK

SEQ ID NO: 133 - protein, 7 aa, Artificial Sequence
  Synthetic
HGSCGSH

SEQ ID NO: 134 - protein, 7 aa, Artificial Sequence
  Synthetic
HGSCGSK

SEQ ID NO: 135 - protein, 7 aa, Artificial Sequence
  Synthetic
HGSCGSR

SEQ ID NO: 136 - protein, 7 aa, Artificial Sequence
  Synthetic
KGSCGSK

SEQ ID NO: 137 - protein, 7 aa, Artificial Sequence
  Synthetic
KGSCGSH

SEQ ID NO: 138 - protein, 7 aa, Artificial Sequence
  Synthetic
KGSCGSR

SEQ ID NO: 139 - protein, 7 aa, Artificial Sequence
  Synthetic
RGSCGSR

SEQ ID NO: 140 - protein, 7 aa, Artificial Sequence
  Synthetic
RGSCGSH

SEQ ID NO: 141 - protein, 7 aa, Artificial Sequence
  Synthetic
RGSCGSK

SEQ ID NO: 142 - protein, 7 aa, Artificial Sequence
  Synthetic
HSGDSGH

SEQ ID NO: 143 - protein, 7 aa, Artificial Sequence
  Synthetic
HSGDSGK

SEQ ID NO: 144 - protein, 7 aa, Artificial Sequence
  Synthetic
HSGDSGR

SEQ ID NO: 145 - protein, 7 aa, Artificial Sequence
  Synthetic
KSGDSGK

SEQ ID NO: 146 - protein, 7 aa, Artificial Sequence
  Synthetic
KSGDSGH

SEQ ID NO: 147 - protein, 7 aa, Artificial Sequence
  Synthetic
KSGDSGR

SEQ ID NO: 148 - protein, 7 aa, Artificial Sequence
  Synthetic
RSGDSGR

SEQ ID NO: 149 - protein, 7 aa, Artificial Sequence
  Synthetic
RSGDSGH

SEQ ID NO: 150 - protein, 7 aa, Artificial Sequence
  Synthetic
RSGDSGK

SEQ ID NO: 151 - protein, 7 aa, Artificial Sequence
  Synthetic
HSGESGH

SEQ ID NO: 152 - protein, 7 aa, Artificial Sequence
  Synthetic
HSGESGK

SEQ ID NO: 153 - protein, 7 aa, Artificial Sequence
  Synthetic
HSGESGR

SEQ ID NO: 154 - protein, 7 aa, Artificial Sequence
  Synthetic
KSGESGK

SEQ ID NO: 155 - protein, 7 aa, Artificial Sequence
  Synthetic
KSGESGH

SEQ ID NO: 156 - protein, 7 aa, Artificial Sequence
  Synthetic
KSGESGR

SEQ ID NO: 157 - protein, 7 aa, Artificial Sequence
  Synthetic
RSGESGR

SEQ ID NO: 158 - protein, 7 aa, Artificial Sequence
  Synthetic
RSGESGH

SEQ ID NO: 159 - protein, 7 aa, Artificial Sequence
  Synthetic
RSGESGK

SEQ ID NO: 160 - protein, 7 aa, Artificial Sequence
  Synthetic
HSGCSGH

SEQ ID NO: 161 - protein, 7 aa, Artificial Sequence
  Synthetic
HSGCSGK

SEQ ID NO: 162 - protein, 7 aa, Artificial Sequence
  Synthetic
HSGCSGR

SEQ ID NO: 163 - protein, 7 aa, Artificial Sequence
  Synthetic
KSGCSGK

SEQ ID NO: 164 - protein, 7 aa, Artificial Sequence
  Synthetic
KSGCSGH

SEQ ID NO: 165 - protein, 7 aa, Artificial Sequence
  Synthetic
KSGCSGR

SEQ ID NO: 166 - protein, 7 aa, Artificial Sequence
  Synthetic
RSGCSGR

SEQ ID NO: 167 - protein, 7 aa, Artificial Sequence
  Synthetic
RSGCSGH

SEQ ID NO: 168 - protein, 7 aa, Artificial Sequence
  Synthetic
RSGCSGK

SEQ ID NO: 169 - protein, 5 aa, Artificial Sequence
  Synthetic
HGDSH

SEQ ID NO: 170 - protein, 5 aa, Artificial Sequence
  Synthetic
HGDSK

SEQ ID NO: 171 - protein, 5 aa, Artificial Sequence
  Synthetic
HGDSR

SEQ ID NO: 172 - protein, 5 aa, Artificial Sequence
  Synthetic
KGDSK

SEQ ID NO: 173 - protein, 5 aa, Artificial Sequence
  Synthetic
KGDSH

SEQ ID NO: 174 - protein, 5 aa, Artificial Sequence
  Synthetic
KGDSR

SEQ ID NO: 175 - protein, 5 aa, Artificial Sequence
  Synthetic
RGDSR

SEQ ID NO: 176 - protein, 5 aa, Artificial Sequence
  Synthetic
RGDSH

SEQ ID NO: 177 - protein, 5 aa, Artificial Sequence
  Synthetic
RGDSK

SEQ ID NO: 178 - protein, 5 aa, Artificial Sequence
  Synthetic
HGESH

SEQ ID NO: 179 - protein, 5 aa, Artificial Sequence
  Synthetic
HGESK

SEQ ID NO: 180 - protein, 5 aa, Artificial Sequence
  Synthetic
HGESR

SEQ ID NO: 181 - protein, 5 aa, Artificial Sequence
  Synthetic
KGESK

SEQ ID NO: 182 - protein, 5 aa, Artificial Sequence
  Synthetic
KGESH

SEQ ID NO: 183 - protein, 5 aa, Artificial Sequence
  Synthetic
KGESR

SEQ ID NO: 184 - protein, 5 aa, Artificial Sequence
  Synthetic
RGESR

SEQ ID NO: 185 - protein, 5 aa, Artificial Sequence
  Synthetic
RGESH

SEQ ID NO: 186 - protein, 5 aa, Artificial Sequence
  Synthetic
RGESK

SEQ ID NO: 187 - protein, 5 aa, Artificial Sequence
  Synthetic
HGCSH

SEQ ID NO: 188 - protein, 5 aa, Artificial Sequence
  Synthetic
HGCSK

SEQ ID NO: 189 - protein, 5 aa, Artificial Sequence
  Synthetic
HGCSR

SEQ ID NO: 190 - protein, 5 aa, Artificial Sequence
  Synthetic
KGCSK

SEQ ID NO: 191 - protein, 5 aa, Artificial Sequence
  Synthetic
KGCSH

SEQ ID NO: 192 - protein, 5 aa, Artificial Sequence
  Synthetic
KGCSR

SEQ ID NO: 193 - protein, 5 aa, Artificial Sequence
  Synthetic
RGCSR

SEQ ID NO: 194 - protein, 5 aa, Artificial Sequence
  Synthetic
RGCSH

SEQ ID NO: 195 - protein, 5 aa, Artificial Sequence
  Synthetic
RGCSK

SEQ ID NO: 196 - protein, 5 aa, Artificial Sequence
  Synthetic
HSDGH

SEQ ID NO: 197 - protein, 5 aa, Artificial Sequence
  Synthetic
HSDGK

SEQ ID NO: 198 - protein, 5 aa, Artificial Sequence
  Synthetic
HSDGR

SEQ ID NO: 199 - protein, 5 aa, Artificial Sequence
  Synthetic
KSDGK

SEQ ID NO: 200 - protein, 5 aa, Artificial Sequence
  Synthetic
KSDGH

SEQ ID NO: 201 - protein, 5 aa, Artificial Sequence
  Synthetic
KSDGR

SEQ ID NO: 202 - protein, 5 aa, Artificial Sequence
  Synthetic
RSDGR

SEQ ID NO: 203 - protein, 5 aa, Artificial Sequence
  Synthetic
RSDGH

SEQ ID NO: 204 - protein, 5 aa, Artificial Sequence
  Synthetic
RSDGK

SEQ ID NO: 205 - protein, 5 aa, Artificial Sequence
  Synthetic
HSEGH

SEQ ID NO: 206 - protein, 5 aa, Artificial Sequence
  Synthetic
HSEGK

SEQ ID NO: 207 - protein, 5 aa, Artificial Sequence
  Synthetic
HSEGR

SEQ ID NO: 208 - protein, 5 aa, Artificial Sequence
  Synthetic
KSEGK

SEQ ID NO: 209 - protein, 5 aa, Artificial Sequence
  Synthetic
KSEGH

SEQ ID NO: 210 - protein, 5 aa, Artificial Sequence
  Synthetic
KSEGR

SEQ ID NO: 211 - protein, 5 aa, Artificial Sequence
  Synthetic
RSEGR

SEQ ID NO: 212 - protein, 5 aa, Artificial Sequence
  Synthetic
RSEGH

SEQ ID NO: 213 - protein, 5 aa, Artificial Sequence
  Synthetic
RSEGK

SEQ ID NO: 214 - protein, 5 aa, Artificial Sequence
  Synthetic
HSCGH

SEQ ID NO: 215 - protein, 5 aa, Artificial Sequence
  Synthetic
HSCGK

SEQ ID NO: 216 - protein, 5 aa, Artificial Sequence
  Synthetic
HSCGR

SEQ ID NO: 217 - protein, 5 aa, Artificial Sequence
  Synthetic
KSCGK

SEQ ID NO: 218 - protein, 5 aa, Artificial Sequence
  Synthetic
KSCGH

SEQ ID NO: 219 - protein, 5 aa, Artificial Sequence
  Synthetic
KSCGR

SEQ ID NO: 220 - protein, 5 aa, Artificial Sequence
  Synthetic
RSCGR

SEQ ID NO: 221 - protein, 5 aa, Artificial Sequence
  Synthetic
RSCGH

SEQ ID NO: 222 - protein, 5 aa, Artificial Sequence
  Synthetic
RSCGK

SEQ ID NO: 223 - protein, 43 aa, Artificial Sequence
  Synthetic
DXXXXXXXXXXXXXXXXXXXXXXXXXXXXXXXXXXXXXXXXXD

SEQ ID NO: 224 - protein, 43 aa, Artificial Sequence
  Synthetic
DXXXXXXXXXXXXXXXXXXXXXXXXXXXXXXXXXXXXXXXXXE

SEQ ID NO: 225 - protein, 43 aa, Artificial Sequence
  Synthetic
DXXXXXXXXXXXXXXXXXXXXXXXXXXXXXXXXXXXXXXXXXC

SEQ ID NO: 226 - protein, 43 aa, Artificial Sequence
  Synthetic
EXXXXXXXXXXXXXXXXXXXXXXXXXXXXXXXXXXXXXXXXXD

SEQ ID NO: 227 - protein, 43 aa, Artificial Sequence
  Synthetic
EXXXXXXXXXXXXXXXXXXXXXXXXXXXXXXXXXXXXXXXXXE

SEQ ID NO: 228 - protein, 43 aa, Artificial Sequence
  Synthetic
EXXXXXXXXXXXXXXXXXXXXXXXXXXXXXXXXXXXXXXXXXC

SEQ ID NO: 229 - protein, 43 aa, Artificial Sequence
  Synthetic
CXXXXXXXXXXXXXXXXXXXXXXXXXXXXXXXXXXXXXXXXXD

SEQ ID NO: 230 - protein, 43 aa, Artificial Sequence
  Synthetic
CXXXXXXXXXXXXXXXXXXXXXXXXXXXXXXXXXXXXXXXXXE

SEQ ID NO: 231 - protein, 43 aa, Artificial Sequence
  Synthetic
CXXXXXXXXXXXXXXXXXXXXXXXXXXXXXXXXXXXXXXXXXC

SEQ ID NO: 232 - protein, 5 aa, Artificial Sequence
  Synthetic
DGHGD

SEQ ID NO: 233 - protein, 5 aa, Artificial Sequence
  Synthetic
DGHGE

SEQ ID NO: 234 - protein, 5 aa, Artificial Sequence
  Synthetic
DGHGC

SEQ ID NO: 235 - protein, 5 aa, Artificial Sequence
  Synthetic
EGHGD

SEQ ID NO: 236 - protein, 5 aa, Artificial Sequence
  Synthetic
EGHGE

SEQ ID NO: 237 - protein, 5 aa, Artificial Sequence
  Synthetic
EGHGC

SEQ ID NO: 238 - protein, 5 aa, Artificial Sequence
  Synthetic
CGHGD

SEQ ID NO: 239 - protein, 5 aa, Artificial Sequence
  Synthetic
CGHGE

SEQ ID NO: 240 - protein, 5 aa, Artificial Sequence
  Synthetic
CGHGC

SEQ ID NO: 241 - protein, 5 aa, Artificial Sequence
  Synthetic
DGKGD

SEQ ID NO: 242 - protein, 5 aa, Artificial Sequence
  Synthetic
DGKGE

SEQ ID NO: 243 - protein, 5 aa, Artificial Sequence
  Synthetic
DGKGC

SEQ ID NO: 244 - protein, 5 aa, Artificial Sequence
  Synthetic
EGKGD

SEQ ID NO: 245 - protein, 5 aa, Artificial Sequence
  Synthetic
EGKGE

SEQ ID NO: 246 - protein, 5 aa, Artificial Sequence
  Synthetic
EGKGC

SEQ ID NO: 247 - protein, 5 aa, Artificial Sequence
  Synthetic
CGKGD

SEQ ID NO: 248 - protein, 5 aa, Artificial Sequence
  Synthetic
CGKGE

SEQ ID NO: 249 - protein, 5 aa, Artificial Sequence
  Synthetic
CGKGC

SEQ ID NO: 250 - protein, 5 aa, Artificial Sequence
  Synthetic
DGRGD

SEQ ID NO: 251 - protein, 5 aa, Artificial Sequence
  Synthetic
DGRGE

SEQ ID NO: 252 - protein, 5 aa, Artificial Sequence
  Synthetic
DGRGC

SEQ ID NO: 253 - protein, 5 aa, Artificial Sequence
  Synthetic
EGRGD

SEQ ID NO: 254 - protein, 5 aa, Artificial Sequence
  Synthetic
EGRGE

SEQ ID NO: 255 - protein, 5 aa, Artificial Sequence
  Synthetic
EGRGC

SEQ ID NO: 256 - protein, 5 aa, Artificial Sequence
  Synthetic
CGRGD

SEQ ID NO: 257 - protein, 5 aa, Artificial Sequence
  Synthetic
CGRGE

SEQ ID NO: 258 - protein, 5 aa, Artificial Sequence
  Synthetic
CGRGC

SEQ ID NO: 259 - protein, 43 aa, Artificial Sequence; identical to SEQ ID NO: 223
  Synthetic

SEQ ID NO: 260 - protein, 43 aa, Artificial Sequence; identical to SEQ ID NO: 224
  Synthetic

SEQ ID NO: 261 - protein, 43 aa, Artificial Sequence; identical to SEQ ID NO: 225
  Synthetic

SEQ ID NO: 262 - protein, 43 aa, Artificial Sequence; identical to SEQ ID NO: 226
  Synthetic

SEQ ID NO: 263 - protein, 43 aa, Artificial Sequence; identical to SEQ ID NO: 227
  Synthetic

SEQ ID NO: 264 - protein, 43 aa, Artificial Sequence; identical to SEQ ID NO: 228
  Synthetic

SEQ ID NO: 265 - protein, 43 aa, Artificial Sequence; identical to SEQ ID NO: 229
  Synthetic

SEQ ID NO: 266 - protein, 43 aa, Artificial Sequence; identical to SEQ ID NO: 230
  Synthetic

SEQ ID NO: 267 - protein, 43 aa, Artificial Sequence; identical to SEQ ID NO: 231
  Synthetic

SEQ ID NO: 268 - protein, 7 aa, Artificial Sequence
  Synthetic
DGGHGGD

SEQ ID NO: 269 - protein, 7 aa, Artificial Sequence
  Synthetic
DGGHGGE

SEQ ID NO: 270 - protein, 7 aa, Artificial Sequence
  Synthetic
DGGHGGC

SEQ ID NO: 271 - protein, 7 aa, Artificial Sequence
  Synthetic
EGGHGGD

SEQ ID NO: 272 - protein, 7 aa, Artificial Sequence
  Synthetic
EGGHGGE

SEQ ID NO: 273 - protein, 7 aa, Artificial Sequence
  Synthetic
EGGHGGC

SEQ ID NO: 274 - protein, 7 aa, Artificial Sequence
  Synthetic
CGGHGGD

SEQ ID NO: 275 - protein, 7 aa, Artificial Sequence
  Synthetic
CGGHGGE

SEQ ID NO: 276 - protein, 7 aa, Artificial Sequence
  Synthetic
CGGHGGC

SEQ ID NO: 277 - protein, 7 aa, Artificial Sequence
  Synthetic
DGGKGGD

SEQ ID NO: 278 - protein, 7 aa, Artificial Sequence
  Synthetic
DGGKGGE

SEQ ID NO: 279 - protein, 7 aa, Artificial Sequence
  Synthetic
DGGKGGC

SEQ ID NO: 280 - protein, 7 aa, Artificial Sequence
  Synthetic
EGGKGGD

SEQ ID NO: 281 - protein, 7 aa, Artificial Sequence
  Synthetic
EGGKGGE

SEQ ID NO: 282 - protein, 7 aa, Artificial Sequence
  Synthetic
EGGKGGC

SEQ ID NO: 283 - protein, 7 aa, Artificial Sequence
  Synthetic
CGGKGGD

SEQ ID NO: 284 - protein, 7 aa, Artificial Sequence
  Synthetic
CGGKGGE

SEQ ID NO: 285 - protein, 7 aa, Artificial Sequence
  Synthetic
CGGKGGC

SEQ ID NO: 286 - protein, 7 aa, Artificial Sequence
  Synthetic
DGGRGGD

SEQ ID NO: 287 - protein, 7 aa, Artificial Sequence
  Synthetic
DGGRGGE

SEQ ID NO: 288 - protein, 7 aa, Artificial Sequence
  Synthetic
DGGRGGC

SEQ ID NO: 289 - protein, 7 aa, Artificial Sequence
  Synthetic
EGGRGGD

SEQ ID NO: 290 - protein, 7 aa, Artificial Sequence
  Synthetic
EGGRGGE

SEQ ID NO: 291 - protein, 7 aa, Artificial Sequence
  Synthetic
EGGRGGC

SEQ ID NO: 292 - protein, 7 aa, Artificial Sequence
  Synthetic
CGGRGGD

SEQ ID NO: 293 - protein, 7 aa, Artificial Sequence
  Synthetic
CGGRGGE

SEQ ID NO: 294 - protein, 7 aa, Artificial Sequence
  Synthetic
CGGRGGC

SEQ ID NO: 295 - protein, 5 aa, Artificial Sequence
  Synthetic
DGHSD

SEQ ID NO: 296 - protein, 5 aa, Artificial Sequence
  Synthetic
DGHSE

SEQ ID NO: 297 - protein, 5 aa, Artificial Sequence
  Synthetic
DGHSC

SEQ ID NO: 298 - protein, 5 aa, Artificial Sequence
  Synthetic
EGHSD

SEQ ID NO: 299 - protein, 5 aa, Artificial Sequence
  Synthetic
EGHSE

SEQ ID NO: 300 - protein, 5 aa, Artificial Sequence
  Synthetic
EGHGE

SEQ ID NO: 301 - protein, 5 aa, Artificial Sequence
  Synthetic
EGHSC

SEQ ID NO: 302 - protein, 5 aa, Artificial Sequence
  Synthetic
CGHSD

SEQ ID NO: 303 - protein, 5 aa, Artificial Sequence
  Synthetic
CGHSE

SEQ ID NO: 304 - protein, 5 aa, Artificial Sequence
  Synthetic
CGHSC

SEQ ID NO: 305 - protein, 5 aa, Artificial Sequence
  Synthetic
DGKSD

SEQ ID NO: 306 - protein, 5 aa, Artificial Sequence
  Synthetic
DGKSE

SEQ ID NO: 307 - protein, 5 aa, Artificial Sequence
  Synthetic
DGKSC

SEQ ID NO: 308 - protein, 5 aa, Artificial Sequence
  Synthetic
EGKSD

SEQ ID NO: 309 - protein, 5 aa, Artificial Sequence
  Synthetic
EGKSE

SEQ ID NO: 310 - protein, 5 aa, Artificial Sequence
  Synthetic
EGKSC

SEQ ID NO: 311 - protein, 5 aa, Artificial Sequence
  Synthetic
CGKSD

SEQ ID NO: 312 - protein, 5 aa, Artificial Sequence
  Synthetic
CGKSE

SEQ ID NO: 313 - protein, 5 aa, Artificial Sequence
  Synthetic
CGKSC

SEQ ID NO: 314 - protein, 5 aa, Artificial Sequence
  Synthetic
DGRSD

SEQ ID NO: 315 - protein, 5 aa, Artificial Sequence
  Synthetic
DGRSE

SEQ ID NO: 316 - protein, 5 aa, Artificial Sequence
  Synthetic
DGRSC

SEQ ID NO: 317 - protein, 5 aa, Artificial Sequence
  Synthetic
EGRSD

SEQ ID NO: 318 - protein, 5 aa, Artificial Sequence
  Synthetic
EGRSE

SEQ ID NO: 319 - protein, 5 aa, Artificial Sequence
  Synthetic
EGRSC

SEQ ID NO: 320 - protein, 5 aa, Artificial Sequence
  Synthetic
CGRSD

SEQ ID NO: 321 - protein, 5 aa, Artificial Sequence
  Synthetic
CGRSE

SEQ ID NO: 322 - protein, 5 aa, Artificial Sequence
  Synthetic
CGRGC

SEQ ID NO: 323 - protein, 7 aa, Artificial Sequence
  Synthetic
DGGHSSD

SEQ ID NO: 324 - protein, 7 aa, Artificial Sequence
  Synthetic
DGGHSSE

SEQ ID NO: 325 - protein, 7 aa, Artificial Sequence
  Synthetic
DGGHSSC

SEQ ID NO: 326 - protein, 7 aa, Artificial Sequence
  Synthetic
EGGHSSD

SEQ ID NO: 327 - protein, 7 aa, Artificial Sequence
  Synthetic
EGGHSSE

SEQ ID NO: 328 - protein, 7 aa, Artificial Sequence
  Synthetic
EGGHSSC

SEQ ID NO: 329 - protein, 7 aa, Artificial Sequence
  Synthetic
CGGHSSD

SEQ ID NO: 330 - protein, 7 aa, Artificial Sequence
  Synthetic
CGGHSSE

SEQ ID NO: 331 - protein, 7 aa, Artificial Sequence
  Synthetic
CGGHSSC

SEQ ID NO: 332 - protein, 7 aa, Artificial Sequence
  Synthetic
DGGKSSD

SEQ ID NO: 333 - protein, 7 aa, Artificial Sequence
  Synthetic
DGGKSSE

SEQ ID NO: 334 - protein, 7 aa, Artificial Sequence
  Synthetic
DGGKSSC

SEQ ID NO: 335 - protein, 7 aa, Artificial Sequence
  Synthetic
EGGKSSD

SEQ ID NO: 336 - protein, 7 aa, Artificial Sequence
  Synthetic
EGGKSSE

SEQ ID NO: 337 - protein, 7 aa, Artificial Sequence
  Synthetic
EGGKSSC

SEQ ID NO: 338 - protein, 7 aa, Artificial Sequence
  Synthetic
CGGKSSD

SEQ ID NO: 339 - protein, 7 aa, Artificial Sequence
  Synthetic
CGGKSSE

SEQ ID NO: 340 - protein, 7 aa, Artificial Sequence
  Synthetic
CGGKSSC

SEQ ID NO: 341 - protein, 7 aa, Artificial Sequence
  Synthetic
DGGRSSD

SEQ ID NO: 342 - protein, 7 aa, Artificial Sequence
  Synthetic
DGGRSSE

SEQ ID NO: 343 - protein, 7 aa, Artificial Sequence
  Synthetic
DGGRSSC

SEQ ID NO: 344 - protein, 7 aa, Artificial Sequence
  Synthetic
EGGRSSD

SEQ ID NO: 345 - protein, 7 aa, Artificial Sequence
  Synthetic
EGGRSSE

SEQ ID NO: 346 - protein, 7 aa, Artificial Sequence
  Synthetic
EGGRSSC

SEQ ID NO: 347 - protein, 7 aa, Artificial Sequence
  Synthetic
CGGRSSD

SEQ ID NO: 348 - protein, 7 aa, Artificial Sequence
  Synthetic
CGGRSSE

SEQ ID NO: 349 - protein, 7 aa, Artificial Sequence
  Synthetic
CGGRSSC

SEQ ID NO: 350 - protein, 5 aa, Artificial Sequence
  Synthetic
HDHDH

SEQ ID NO: 351 - protein, 5 aa, Artificial Sequence
  Synthetic
HEHEH

SEQ ID NO: 352 - protein, 5 aa, Artificial Sequence
  Synthetic
HSHSH

SEQ ID NO: 353 - protein, 5 aa, Artificial Sequence
  Synthetic
HTHTH

SEQ ID NO: 354 - protein, 5 aa, Artificial Sequence
  Synthetic
HNHNH

SEQ ID NO: 355 - protein, 5 aa, Artificial Sequence
  Synthetic
HQHQH

SEQ ID NO: 356 - protein, 5 aa, Artificial Sequence
  Synthetic
HPHPH

SEQ ID NO: 357 - protein, 5 aa, Artificial Sequence
  Synthetic
HGHGH

SEQ ID NO: 358 - protein, 5 aa, Artificial Sequence
  Synthetic
HAHAH

SEQ ID NO: 359 - protein, 5 aa, Artificial Sequence
  Synthetic
HKHKH

SEQ ID NO: 360 - protein, 5 aa, Artificial Sequence
  Synthetic
HRHRH

SEQ ID NO: 361 - protein, 5 aa, Artificial Sequence
  Synthetic
HYHYH

SEQ ID NO: 362 - protein, 5 aa, Artificial Sequence
  Synthetic
HMHMH

SEQ ID NO: 363 - protein, 6 aa, Artificial Sequence
  Synthetic
HGAHGH

SEQ ID NO: 364 - protein, 4 aa, Artificial Sequence
  Synthetic
HGAH

SEQ ID NO: 365 - protein, 4 aa, Artificial Sequence
  Synthetic
AHVH

SEQ ID NO: 366 - protein, 4 aa, Artificial Sequence
  Synthetic
HDDH

SEQ ID NO: 367 - protein, 6 aa, Artificial Sequence
  Synthetic
HDDHDH

SEQ ID NO: 368 - protein, 5 aa, Artificial Sequence
  Synthetic
AHAHA

SEQ ID NO: 369 - protein, 5 aa, Artificial Sequence
  Synthetic
VHVHV

SEQ ID NO: 370 - protein, 5 aa, Artificial Sequence
  Synthetic
LHLHL

SEQ ID NO: 371 - protein, 6 aa, Artificial Sequence
  Synthetic
GHGGHG

SEQ ID NO: 372 - protein, 6 aa, Artificial Sequence
  Synthetic
AHAAHA

SEQ ID NO: 373 - protein, 6 aa, Artificial Sequence
  Synthetic
VHVVHV

SEQ ID NO: 374 - protein, 6 aa, Artificial Sequence
  Synthetic
LHLLHL

SEQ ID NO: 375 - protein, 5 aa, Artificial Sequence
  Synthetic
IHIHI

SEQ ID NO: 376 - protein, 5 aa, Artificial Sequence
  Synthetic
SHSHS

SEQ ID NO: 377 - protein, 5 aa, Artificial Sequence
  Synthetic
THTHT

SEQ ID NO: 378 - protein, 5 aa, Artificial Sequence
  Synthetic
NHNHN

SEQ ID NO: 379 - protein, 5 aa, Artificial Sequence
  Synthetic
QHQHQ

SEQ ID NO: 380 - protein, 5 aa, Artificial Sequence
  Synthetic
KHKHK

SEQ ID NO: 381 - protein, 5 aa, Artificial Sequence
  Synthetic
RHRHR

SEQ ID NO: 382 - protein, 5 aa, Artificial Sequence
  Synthetic
DHDHD

SEQ ID NO: 383 - protein, 5 aa, Artificial Sequence
  Synthetic
EHEHE

SEQ ID NO: 384 - protein, 5 aa, Artificial Sequence
  Synthetic
CHCHC

SEQ ID NO: 385 - protein, 5 aa, Artificial Sequence
  Synthetic
MHMHM

SEQ ID NO: 386 - protein, 5 aa, Artificial Sequence
  Synthetic
FHFHF

SEQ ID NO: 387 - protein, 5 aa, Artificial Sequence
  Synthetic
WHWHW

SEQ ID NO: 388 - protein, 5 aa, Artificial Sequence
  Synthetic
YHYHY

SEQ ID NO: 389 - protein, 5 aa, Artificial Sequence
  Synthetic
PHPHP

SEQ ID NO: 390 - protein, 6 aa, Artificial Sequence
  Synthetic
IHIIHI

SEQ ID NO: 391 - protein, 6 aa, Artificial Sequence
  Synthetic
SHSSHS

SEQ ID NO: 392 - protein, 6 aa, Artificial Sequence
  Synthetic
THTTHT

SEQ ID NO: 393 - protein, 6 aa, Artificial Sequence
  Synthetic
NHNNHN

SEQ ID NO: 394 - protein, 6 aa, Artificial Sequence
  Synthetic
QHQQHQ

SEQ ID NO: 395 - protein, 6 aa, Artificial Sequence
  Synthetic
KHKKHK

SEQ ID NO: 396 - protein, 6 aa, Artificial Sequence
  Synthetic
RHRRHR

SEQ ID NO: 397 - protein, 6 aa, Artificial Sequence
  Synthetic
DHDDHD

SEQ ID NO: 398 - protein, 6 aa, Artificial Sequence
  Synthetic
EHEEHE

SEQ ID NO: 399 - protein, 6 aa, Artificial Sequence
  Synthetic
CHCCHC

SEQ ID NO: 400 - protein, 6 aa, Artificial Sequence
  Synthetic
MHMMHM

SEQ ID NO: 401 - protein, 6 aa, Artificial Sequence
  Synthetic
FHFFHF

SEQ ID NO: 402 - protein, 6 aa, Artificial Sequence
  Synthetic
WHWWHW

SEQ ID NO: 403 - protein, 6 aa, Artificial Sequence
  Synthetic
YHYYHY

SEQ ID NO: 404 - protein, 6 aa, Artificial Sequence
  Synthetic
PHPPHP

SEQ ID NO: 405 - protein, 5 aa, Artificial Sequence
  Synthetic
GHGHX

SEQ ID NO: 406 - protein, 5 aa, Artificial Sequence
  Synthetic
AHAHX

SEQ ID NO: 407 - protein, 5 aa, Artificial Sequence
  Synthetic
VHVHX

SEQ ID NO: 408 - protein, 5 aa, Artificial Sequence
  Synthetic
LHLHX

SEQ ID NO: 409 - protein, 6 aa, Artificial Sequence
  Synthetic
IHIHIX

SEQ ID NO: 410 - protein, 5 aa, Artificial Sequence
  Synthetic
SHSHX

SEQ ID NO: 411 - protein, 5 aa, Artificial Sequence
  Synthetic
THTHX

SEQ ID NO: 412 - protein, 5 aa, Artificial Sequence
  Synthetic
NHNHX

SEQ ID NO: 413 - protein, 5 aa, Artificial Sequence
  Synthetic
QHQHX

SEQ ID NO: 414 - protein, 5 aa, Artificial Sequence
  Synthetic
KHKHX

SEQ ID NO: 415 - protein, 5 aa, Artificial Sequence
  Synthetic
RHRHX

SEQ ID NO: 416 - protein, 5 aa, Artificial Sequence
  Synthetic
DHDHX

SEQ ID NO: 417 - protein, 5 aa, Artificial Sequence
  Synthetic
EHEHX

SEQ ID NO: 418 - protein, 5 aa, Artificial Sequence
  Synthetic
CHCHX

SEQ ID NO: 419 - protein, 5 aa, Artificial Sequence
  Synthetic
MHMHX

SEQ ID NO: 420 - protein, 5 aa, Artificial Sequence
  Synthetic
FHFHX

SEQ ID NO: 421 - protein, 5 aa, Artificial Sequence
  Synthetic
WHWHX

SEQ ID NO: 422 - protein, 5 aa, Artificial Sequence
  Synthetic
YHYHX

SEQ ID NO: 423 - protein, 5 aa, Artificial Sequence
  Synthetic
PHPHX

SEQ ID NO: 424 - protein, 6 aa, Artificial Sequence
  Synthetic
GHGXHG

SEQ ID NO: 425 - protein, 6 aa, Artificial Sequence
  Synthetic
AHAXHA

SEQ ID NO: 426 - protein, 6 aa, Artificial Sequence
  Synthetic
VHVXHV

SEQ ID NO: 427 - protein, 6 aa, Artificial Sequence
  Synthetic
LHLXHL

SEQ ID NO: 428 - protein, 6 aa, Artificial Sequence
  Synthetic
IHIXHI

SEQ ID NO: 429 - protein, 6 aa, Artificial Sequence
  Synthetic
SHSXHS

SEQ ID NO: 430 - protein, 6 aa, Artificial Sequence
  Synthetic
THTXHT

SEQ ID NO: 431 - protein, 6 aa, Artificial Sequence
  Synthetic
NHNXHN

SEQ ID NO: 432 - protein, 6 aa, Artificial Sequence
  Synthetic
QHQXHQ

SEQ ID NO: 433 - protein, 6 aa, Artificial Sequence
  Synthetic
KHKXHK

SEQ ID NO: 434 - protein, 6 aa, Artificial Sequence
  Synthetic
RHRXHR

SEQ ID NO: 435 - protein, 6 aa, Artificial Sequence
  Synthetic
DHDXHD

SEQ ID NO: 436 - protein, 6 aa, Artificial Sequence
  Synthetic
EHEXHE

SEQ ID NO: 437 - protein, 6 aa, Artificial Sequence
  Synthetic
CHCXHC

SEQ ID NO: 438 - protein, 6 aa, Artificial Sequence
  Synthetic
MHMXHM

SEQ ID NO: 439 - protein, 6 aa, Artificial Sequence
  Synthetic
FHFXHF

SEQ ID NO: 440 - protein, 6 aa, Artificial Sequence
  Synthetic
WHWXHW

SEQ ID NO: 441 - protein, 6 aa, Artificial Sequence
  Synthetic
YHYXHY

SEQ ID NO: 442 - protein, 6 aa, Artificial Sequence
  Synthetic
PHPXHP

SEQ ID NO: 443 - protein, 6 aa, Artificial Sequence
  Synthetic
MHDDHD

SEQ ID NO: 444 - protein, 6 aa, Artificial Sequence
  Synthetic
MHDEHD

SEQ ID NO: 445 - protein, 6 aa, Artificial Sequence
  Synthetic
MHDSHD

SEQ ID NO: 446 - protein, 6 aa, Artificial Sequence
  Synthetic
MHDTHD

SEQ ID NO: 447 - protein, 6 aa, Artificial Sequence
  Synthetic
MHDNHD

SEQ ID NO: 448 - protein, 6 aa, Artificial Sequence
  Synthetic
MHDQHD

SEQ ID NO: 449 - protein, 6 aa, Artificial Sequence
  Synthetic
MHDPHD

SEQ ID NO: 450 - protein, 6 aa, Artificial Sequence
  Synthetic
MHDGHD

SEQ ID NO: 451 - protein, 6 aa, Artificial Sequence
  Synthetic
MHDAHD

SEQ ID NO: 452 - protein, 6 aa, Artificial Sequence
  Synthetic
MHDKHD

SEQ ID NO: 453 - protein, 6 aa, Artificial Sequence
  Synthetic
MHDRHD

SEQ ID NO: 454 - protein, 6 aa, Artificial Sequence
  Synthetic
MHDYHD

SEQ ID NO: 455 - protein, 6 aa, Artificial Sequence
  Synthetic
MHEEHE

SEQ ID NO: 456 - protein, 6 aa, Artificial Sequence
  Synthetic
MHEDHE

SEQ ID NO: 457 - protein, 6 aa, Artificial Sequence
  Synthetic
MHESHE

SEQ ID NO: 458 - protein, 6 aa, Artificial Sequence
  Synthetic
MHETHE

SEQ ID NO: 459 - protein, 6 aa, Artificial Sequence
  Synthetic
MHENHE

SEQ ID NO: 460 - protein, 6 aa, Artificial Sequence
  Synthetic
MHEQHE

SEQ ID NO: 461 - protein, 6 aa, Artificial Sequence
  Synthetic
MHEPHE

SEQ ID NO: 462 - protein, 6 aa, Artificial Sequence
  Synthetic
MHEGHE

SEQ ID NO: 463 - protein, 6 aa, Artificial Sequence
  Synthetic
MHEAHE

SEQ ID NO: 464 - protein, 6 aa, Artificial Sequence
  Synthetic
MHEKHE

SEQ ID NO: 465 - protein, 6 aa, Artificial Sequence
  Synthetic
MHERHE

SEQ ID NO: 466 - protein, 6 aa, Artificial Sequence
  Synthetic
MHEYHE

SEQ ID NO: 467 - protein, 6 aa, Artificial Sequence
  Synthetic
MHSSHS

SEQ ID NO: 468 - protein, 6 aa, Artificial Sequence
  Synthetic
MHSDHS

SEQ ID NO: 469 - protein, 6 aa, Artificial Sequence
  Synthetic
MHSEHS

SEQ ID NO: 470 - protein, 6 aa, Artificial Sequence
  Synthetic
MHSTHS

SEQ ID NO: 471 - protein, 6 aa, Artificial Sequence
  Synthetic
MHSNHS

SEQ ID NO: 472 - protein, 6 aa, Artificial Sequence
  Synthetic
MHSQHS

SEQ ID NO: 473 - protein, 6 aa, Artificial Sequence
  Synthetic
MHSPHS

SEQ ID NO: 474 - protein, 6 aa, Artificial Sequence
  Synthetic
MHSGHS

SEQ ID NO: 475 - protein, 6 aa, Artificial Sequence
  Synthetic
MHSAHS

SEQ ID NO: 476 - protein, 6 aa, Artificial Sequence
  Synthetic
MHSKHS

SEQ ID NO: 477 - protein, 6 aa, Artificial Sequence
  Synthetic
MHSRHS

SEQ ID NO: 478 - protein, 6 aa, Artificial Sequence
  Synthetic
MHSYHS

SEQ ID NO: 479 - protein, 6 aa, Artificial Sequence
  Synthetic
MHTTHT

SEQ ID NO: 480 - protein, 6 aa, Artificial Sequence
  Synthetic
MHTEHT

SEQ ID NO: 481 - protein, 6 aa, Artificial Sequence
  Synthetic
MHTSHT

SEQ ID NO: 482 - protein, 6 aa, Artificial Sequence
  Synthetic
MHTDHT

SEQ ID NO: 483 - protein, 6 aa, Artificial Sequence
  Synthetic
MHTNHT

SEQ ID NO: 484 - protein, 6 aa, Artificial Sequence
  Synthetic
MHTQHT

SEQ ID NO: 485 - protein, 6 aa, Artificial Sequence
  Synthetic
MHTPHT

SEQ ID NO: 486 - protein, 6 aa, Artificial Sequence
  Synthetic
MHTGHT

SEQ ID NO: 487 - protein, 6 aa, Artificial Sequence
  Synthetic
MHTAHT

SEQ ID NO: 488 - protein, 6 aa, Artificial Sequence
  Synthetic
MHTKHT

SEQ ID NO: 489 - protein, 6 aa, Artificial Sequence
  Synthetic
MHTRHT

SEQ ID NO: 490 - protein, 6 aa, Artificial Sequence
  Synthetic
MHTYHT

SEQ ID NO: 491 - protein, 6 aa, Artificial Sequence
  Synthetic
MHNNHN

SEQ ID NO: 492 - protein, 6 aa, Artificial Sequence
  Synthetic
MHNDHN

SEQ ID NO: 493 - protein, 6 aa, Artificial Sequence
  Synthetic
MHNEHN

SEQ ID NO: 494 - protein, 6 aa, Artificial Sequence
  Synthetic
MHNSHN

SEQ ID NO: 495 - protein, 6 aa, Artificial Sequence
  Synthetic
MHNTHN

SEQ ID NO: 496 - protein, 6 aa, Artificial Sequence
  Synthetic
MHNQHN

SEQ ID NO: 497 - protein, 6 aa, Artificial Sequence
  Synthetic
MHNPHN

SEQ ID NO: 498 - protein, 6 aa, Artificial Sequence
  Synthetic
MHNGHN

SEQ ID NO: 499 - protein, 6 aa, Artificial Sequence
  Synthetic
MHNAHN

SEQ ID NO: 500 - protein, 6 aa, Artificial Sequence
  Synthetic
MHNKHN

SEQ ID NO: 501 - protein, 6 aa, Artificial Sequence
  Synthetic
MHNRHN

SEQ ID NO: 502 - protein, 6 aa, Artificial Sequence
  Synthetic
MHNYHN

SEQ ID NO: 503 - protein, 6 aa, Artificial Sequence
  Synthetic
MHQQHQ

SEQ ID NO: 504 - protein, 6 aa, Artificial Sequence
  Synthetic
MHQDHQ

SEQ ID NO: 505 - protein, 6 aa, Artificial Sequence
  Synthetic
MHQEHQ

SEQ ID NO: 506 - protein, 6 aa, Artificial Sequence
  Synthetic
MHQSHQ

SEQ ID NO: 507 - protein, 6 aa, Artificial Sequence
  Synthetic
MHQTHQ

SEQ ID NO: 508 - protein, 6 aa, Artificial Sequence
  Synthetic
MHQNHQ

SEQ ID NO: 509 - protein, 6 aa, Artificial Sequence
  Synthetic
MHQPHQ

SEQ ID NO: 510 - protein, 6 aa, Artificial Sequence
  Synthetic
MHQGHQ

SEQ ID NO: 511 - protein, 6 aa, Artificial Sequence
  Synthetic
MHQAHQ

SEQ ID NO: 512 - protein, 6 aa, Artificial Sequence
  Synthetic
MHQKHQ

SEQ ID NO: 513 - protein, 6 aa, Artificial Sequence
  Synthetic
MHQRHQ

SEQ ID NO: 514 - protein, 6 aa, Artificial Sequence
  Synthetic
MHQHYQ

SEQ ID NO: 515 - protein, 6 aa, Artificial Sequence
  Synthetic
MHPPHP

SEQ ID NO: 516 - protein, 6 aa, Artificial Sequence
  Synthetic
MHPDHP

SEQ ID NO: 517 - protein, 6 aa, Artificial Sequence
  Synthetic
MHPEHP

SEQ ID NO: 518 - protein, 6 aa, Artificial Sequence
  Synthetic
MHPSHP

SEQ ID NO: 519 - protein, 6 aa, Artificial Sequence
  Synthetic
MHPTHP

SEQ ID NO: 520 - protein, 6 aa, Artificial Sequence
  Synthetic
MHPNHP

SEQ ID NO: 521 - protein, 6 aa, Artificial Sequence
  Synthetic
MHPQHP

SEQ ID NO: 522 - protein, 6 aa, Artificial Sequence
  Synthetic
MHPGHP

SEQ ID NO: 523 - protein, 6 aa, Artificial Sequence
  Synthetic
MHPAHP

SEQ ID NO: 524 - protein, 6 aa, Artificial Sequence
  Synthetic
MHPKHP

SEQ ID NO: 525 - protein, 6 aa, Artificial Sequence
  Synthetic
MHPRHP

SEQ ID NO: 526 - protein, 6 aa, Artificial Sequence
  Synthetic
MHPYHP

SEQ ID NO: 527 - protein, 6 aa, Artificial Sequence
  Synthetic
MHGGHG

SEQ ID NO: 528 - protein, 6 aa, Artificial Sequence
  Synthetic
MHGDHG

SEQ ID NO: 529 - protein, 6 aa, Artificial Sequence
  Synthetic
MHGEHG

SEQ ID NO: 530 - protein, 6 aa, Artificial Sequence
  Synthetic
MHGSHG

SEQ ID NO: 531 - protein, 6 aa, Artificial Sequence
  Synthetic
MHGTHG

SEQ ID NO: 532 - protein, 6 aa, Artificial Sequence
  Synthetic
MHGNHG

SEQ ID NO: 533 - protein, 6 aa, Artificial Sequence
  Synthetic
MHGQHG

SEQ ID NO: 534 - protein, 6 aa, Artificial Sequence
  Synthetic
MHGPHG

SEQ ID NO: 535 - protein, 6 aa, Artificial Sequence
  Synthetic
MHGAHG

SEQ ID NO: 536 - protein, 6 aa, Artificial Sequence
  Synthetic
MHGKHG

SEQ ID NO: 537 - protein, 6 aa, Artificial Sequence
  Synthetic
MHGRHG

SEQ ID NO: 538 - protein, 6 aa, Artificial Sequence
  Synthetic
MHGYHG

SEQ ID NO: 539 - protein, 6 aa, Artificial Sequence
  Synthetic
MHAAHA

SEQ ID NO: 540 - protein, 6 aa, Artificial Sequence
  Synthetic
MHADHA

SEQ ID NO: 541 - protein, 6 aa, Artificial Sequence
  Synthetic
MHAEHA

SEQ ID NO: 542 - protein, 6 aa, Artificial Sequence
  Synthetic
MHASHA

SEQ ID NO: 543 - protein, 6 aa, Artificial Sequence
  Synthetic
MHATHA

SEQ ID NO: 544 - protein, 6 aa, Artificial Sequence
  Synthetic
MHANHA

SEQ ID NO: 545 - protein, 6 aa, Artificial Sequence
  Synthetic
MHAQHA

SEQ ID NO: 546 - protein, 6 aa, Artificial Sequence
  Synthetic
MHAPHA

SEQ ID NO: 547 - protein, 6 aa, Artificial Sequence
  Synthetic
MHAGHA

SEQ ID NO: 548 - protein, 6 aa, Artificial Sequence
  Synthetic
MHAKHA

SEQ ID NO: 549 - protein, 6 aa, Artificial Sequence
  Synthetic
MHARHA

SEQ ID NO: 550 - protein, 6 aa, Artificial Sequence
  Synthetic
MHAYHA

SEQ ID NO: 551 - protein, 6 aa, Artificial Sequence
  Synthetic
MHKKHK

SEQ ID NO: 552 - protein, 6 aa, Artificial Sequence
  Synthetic
MHKDHK

SEQ ID NO: 553 - protein, 6 aa, Artificial Sequence
  Synthetic
MHKEHK

SEQ ID NO: 554 - protein, 6 aa, Artificial Sequence
  Synthetic
MHKSHK

SEQ ID NO: 555 - protein, 6 aa, Artificial Sequence
  Synthetic
MHKTHK

SEQ ID NO: 556 - protein, 6 aa, Artificial Sequence
  Synthetic
MHKNHK

SEQ ID NO: 557 - protein, 6 aa, Artificial Sequence
  Synthetic
MHKQHK

SEQ ID NO: 558 - protein, 6 aa, Artificial Sequence
  Synthetic
MHKPHK

SEQ ID NO: 559 - protein, 6 aa, Artificial Sequence
  Synthetic
MHKGHK

SEQ ID NO: 560 - protein, 6 aa, Artificial Sequence
  Synthetic
MHKAHK

SEQ ID NO: 561 - protein, 6 aa, Artificial Sequence
  Synthetic
MHKRHK

SEQ ID NO: 562 - protein, 6 aa, Artificial Sequence
  Synthetic
MHKYHK

SEQ ID NO: 563 - protein, 6 aa, Artificial Sequence
  Synthetic
MHRRHR

SEQ ID NO: 564 - protein, 6 aa, Artificial Sequence
  Synthetic
MHRDHR

SEQ ID NO: 565 - protein, 6 aa, Artificial Sequence
  Synthetic
MHREHR

SEQ ID NO: 566 - protein, 6 aa, Artificial Sequence
  Synthetic
MHRSHR

SEQ ID NO: 567 - protein, 6 aa, Artificial Sequence
  Synthetic
MHRTHR

SEQ ID NO: 568 - protein, 6 aa, Artificial Sequence
  Synthetic
MHRNHR

SEQ ID NO: 569 - protein, 6 aa, Artificial Sequence
  Synthetic
MHRQHR

SEQ ID NO: 570 - protein, 6 aa, Artificial Sequence
  Synthetic
MHRPHR

SEQ ID NO: 571 - protein, 6 aa, Artificial Sequence
  Synthetic
MHRGHR

SEQ ID NO: 572 - protein, 6 aa, Artificial Sequence
  Synthetic
MHRAHR

SEQ ID NO: 573 - protein, 6 aa, Artificial Sequence
  Synthetic
MHRKHR

SEQ ID NO: 574 - protein, 6 aa, Artificial Sequence
  Synthetic
MHRYHR

SEQ ID NO: 575 - protein, 6 aa, Artificial Sequence
  Synthetic
MHYYHY

SEQ ID NO: 576 - protein, 6 aa, Artificial Sequence
  Synthetic
MHYDHY

SEQ ID NO: 577 - protein, 6 aa, Artificial Sequence
  Synthetic
MHYEHY

SEQ ID NO: 578 - protein, 6 aa, Artificial Sequence
  Synthetic
MHYSHY

SEQ ID NO: 579 - protein, 6 aa, Artificial Sequence
  Synthetic
MHYTHY

SEQ ID NO: 580 - protein, 6 aa, Artificial Sequence
  Synthetic
MHYNHY

SEQ ID NO: 581 - protein, 6 aa, Artificial Sequence
  Synthetic
MHYQHY

SEQ ID NO: 582 - protein, 6 aa, Artificial Sequence
  Synthetic
MHYPHY

SEQ ID NO: 583 - protein, 6 aa, Artificial Sequence
  Synthetic
MHYGHY

SEQ ID NO: 584 - protein, 6 aa, Artificial Sequence
  Synthetic
MHYAHY

SEQ ID NO: 585 - protein, 6 aa, Artificial Sequence
  Synthetic
MHYKHY

SEQ ID NO: 586 - protein, 6 aa, Artificial Sequence
  Synthetic
MHYRHY

SEQ ID NO: 587 - protein, 4 aa, Artificial Sequence
  Synthetic
GAKG

SEQ ID NO: 588 - protein, 4 aa, Artificial Sequence
  Synthetic
GARG

SEQ ID NO: 589 - protein, 5 aa, Artificial Sequence
  Synthetic
MHYHY

SEQ ID NO: 590 - protein, 5 aa, Artificial Sequence
  Synthetic
MHQHQ

SEQ ID NO: 591 - protein, 5 aa, Artificial Sequence
  Synthetic
MHPHP

SEQ ID NO: 592 - protein, 5 aa, Artificial Sequence
  Synthetic
MHGHG

SEQ ID NO: 593 - protein, 5 aa, Artificial Sequence
  Synthetic
MHAHA

SEQ ID NO: 594 - protein, 5 aa, Artificial Sequence
  Synthetic
MHKHK

SEQ ID NO: 595 - protein, 5 aa, Artificial Sequence
  Synthetic
MHRHR

SEQ ID NO: 596 - protein, 5 aa, Artificial Sequence
  Synthetic
MHNHN

SEQ ID NO: 597 - protein, 5 aa, Artificial Sequence
  Synthetic
MHDHD

SEQ ID NO: 598 - protein, 5 aa, Artificial Sequence
  Synthetic
MHEHE

SEQ ID NO: 599 - protein, 5 aa, Artificial Sequence
  Synthetic
MHSHS

SEQ ID NO: 600 - protein, 5 aa, Artificial Sequence
  Synthetic
MHTHT

SEQ ID NO: 601 - protein, 6 aa, Artificial Sequence
  Synthetic
HGAKGH

SEQ ID NO: 602 - protein, 6 aa, Artificial Sequence
  Synthetic
HGARGH

SEQ ID NO: 603 - protein, 5 aa, Artificial Sequence
  Synthetic
MDHDH

SEQ ID NO: 604 - protein, 5 aa, Artificial Sequence
  Synthetic
GSHDH

SEQ ID NO: 605 - protein, 5 aa, Artificial Sequence
  Synthetic
GSHGH

SEQ ID NO: 606 - protein, 8 aa, Artificial Sequence
  Synthetic
EHGMGHNT

SEQ ID NO: 607 - protein, 196 aa, Artificial Sequence
  SlyD protein
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 608 - protein, 48 aa, Artificial Sequence
  SlyDC1 protein
HGHVHGAHDHHHDHDHDGCCGGHGHDHGHEHGGEGCCGGKGNGGCGCH

SEQ ID NO: 609 - protein, 17 aa, Artificial Sequence
  SlyDC2 protein
HGHVHGAHDHHHDHDHD

SEQ ID NO: 610 - protein, 10 aa, Artificial Sequence
  SlyDC3 protein
HVHGAHDHHH

SEQ ID NO: 611 - protein, 10 aa, Artificial Sequence
  SlyDC4 protein
HVHGAHDHHH

SEQ ID NO: 612 - protein, 7 aa, Artificial Sequence
  SlyDC5 protein
HVHGAHD

SEQ ID NO: 613 - protein, 4 aa, Artificial Sequence
  SlyDC6 protein
HDHH

SEQ ID NO: 614 - protein, 7 aa, Artificial Sequence
  SlyDC7 protein
HGAHDHH

SEQ ID NO: 615 - protein, 302 aa, Artificial Sequence
  Bradyrhizobium japonicum USDA 110 HypB protein
MCTVCGCSDGKASIEHAHDHHHDHGHDHDHGHDGHHHHHHGHDQDHHHHHDHAHGDAGLLDCGANPAGQKIAGMSSDRIIQVERDILGKNDRLAADNRARFRADEVLAFNLVSSPGAGKTSLLVRAVSELKDSFAIGVIEGDQQTSNDAERIRATGVPAIQVNTGKGCHLDAAMVGEAYDRLPWLNGGLLFIENVGNLVCPAAFDLGEACKIVVFSTTEGEDKPLKYPDMFAASSLMLINKIDLASVLDFDLARTIEYARRVNPKIEVLTLSARTGEGFAAFYAWIRKRMAATTPAAMTAAE

SEQ ID NO: 616 - protein, 4 aa, Artificial Sequence
  Protease cleavage site
IEGR

SEQ ID NO: 617 - protein, 4 aa, Artificial Sequence
  Protease cleavage site
LVPR

SEQ ID NO: 618 - protein, 7 aa, Artificial Sequence
  Protease cleavage site
EXXYXQX

SEQ ID NO: 619 - protein, 7 aa, Artificial Sequence
  Protease cleavage site
ENLYFQG

SEQ ID NO: 620 - protein, 4 aa, Artificial Sequence
  Caspase cleavage site
DEXD

SEQ ID NO: 621 - protein, 7 aa, Artificial Sequence
  Synthetic
HGGEGGH

SEQ ID NO: 622 - protein, 6 aa, Artificial Sequence
  Synthetic
HHHHHH

SEQ ID NO: 623 - protein, 5 aa, Artificial Sequence
  Synthetic
SHDHG

SEQ ID NO: 624 - protein, 5 aa, Artificial Sequence
  Synthetic
HDGHS

SEQ ID NO: 625 - protein, 6 aa, Artificial Sequence
  Synthetic
THDGTH

SEQ ID NO: 626 - protein, 4 aa, Artificial Sequence
  Synthetic
HDHG

SEQ ID NO: 627 - protein, 5 aa, Artificial Sequence
  Synthetic
SHDGH

SEQ ID NO: 628 - protein, 7 aa, Artificial Sequence
  Synthetic
HGAHDHG

SEQ ID NO: 629 - protein, 5 aa, Artificial Sequence
  Synthetic
THDGH

SEQ ID NO: 630 - protein, 6 aa, Artificial Sequence
  Synthetic
GSHGAH

SEQ ID NO: 631 - protein, 6 aa, Artificial Sequence
  Synthetic
GSHDHG

SEQ ID NO: 632 - protein, 5 aa, Artificial Sequence
  Synthetic
HDGHT

SEQ ID NO: 633 - protein, 6 aa, Artificial Sequence
  Synthetic
SHDGSH

SEQ ID NO: 634 - protein, 6 aa, Artificial Sequence
  Synthetic
MHEDHD

SEQ ID NO: 635 - protein, 6 aa, Artificial Sequence
  Synthetic
MHESHD

SEQ ID NO: 636 - protein, 6 aa, Artificial Sequence
  Synthetic
MHETHD

SEQ ID NO: 637 - protein, 6 aa, Artificial Sequence
  Synthetic
MHENHD

SEQ ID NO: 638 - protein, 6 aa, Artificial Sequence
  Synthetic
MHEQHD

SEQ ID NO: 639 - protein, 6 aa, Artificial Sequence
  Synthetic
MHEPHD

SEQ ID NO: 640 - protein, 6 aa, Artificial Sequence
  Synthetic
MHEGHD

SEQ ID NO: 641 - protein, 6 aa, Artificial Sequence
  Synthetic
MHEAHD

SEQ ID NO: 642 - protein, 6 aa, Artificial Sequence
  Synthetic
MHEKHD

SEQ ID NO: 643 - protein, 6 aa, Artificial Sequence
  Synthetic
MHERHD

SEQ ID NO: 644 - protein, 6 aa, Artificial Sequence
  Synthetic
MHEYHD

SEQ ID NO: 645 - protein, 6 aa, Artificial Sequence
  Synthetic
MHSDHD

SEQ ID NO: 646 - protein, 6 aa, Artificial Sequence
  Synthetic
MHSEHD

SEQ ID NO: 647 - protein, 6 aa, Artificial Sequence
  Synthetic
MHSTHD

SEQ ID NO: 648 - protein, 6 aa, Artificial Sequence
  Synthetic
MHSNHD

SEQ ID NO: 649 - protein, 6 aa, Artificial Sequence
  Synthetic
MHSQHD

SEQ ID NO: 650 - protein, 6 aa, Artificial Sequence
  Synthetic
MHSPHD

SEQ ID NO: 651 - protein, 6 aa, Artificial Sequence
  Synthetic
MHSGHD

SEQ ID NO: 652 - protein, 6 aa, Artificial Sequence
  Synthetic
MHSAHD

SEQ ID NO: 653 - protein, 6 aa, Artificial Sequence
  Synthetic
MHSKHD

SEQ ID NO: 654 - protein, 6 aa, Artificial Sequence
  Synthetic
MHSRHD

SEQ ID NO: 655 - protein, 6 aa, Artificial Sequence
  Synthetic
MHSYHD

SEQ ID NO: 656 - protein, 6 aa, Artificial Sequence
  Synthetic
MHTSHS

SEQ ID NO: 657 - protein, 6 aa, Artificial Sequence
  Synthetic
MHTDHD

SEQ ID NO: 658 - protein, 6 aa, Artificial Sequence
  Synthetic
MHTNHD

SEQ ID NO: 659 - protein, 6 aa, Artificial Sequence
  Synthetic
MHTQHD

SEQ ID NO: 660 - protein, 6 aa, Artificial Sequence
  Synthetic
MHTPHD

SEQ ID NO: 661 - protein, 6 aa, Artificial Sequence
  Synthetic
MHTGHD

SEQ ID NO: 662 - protein, 6 aa, Artificial Sequence
  Synthetic
MHTAHD

SEQ ID NO: 663 - protein, 6 aa, Artificial Sequence
  Synthetic
MHTKHD

SEQ ID NO: 664 - protein, 6 aa, Artificial Sequence
  Synthetic
MHTRHD

SEQ ID NO: 665 - protein, 6 aa, Artificial Sequence
  Synthetic
MHTYHD

SEQ ID NO: 666 - protein, 6 aa, Artificial Sequence
  Synthetic
HVHGAH

SEQ ID NO: 667 - protein, 6 aa, Artificial Sequence
  Synthetic
HDHDDH

SEQ ID NO: 668 - protein, 4 aa, Artificial Sequence
  Synthetic
HDRG

SEQ ID NO: 669 - protein, 4 aa, Artificial Sequence
  Synthetic
HDKG

SEQ ID NO: 670 - protein, 7 aa, Artificial Sequence
  Synthetic
MHKSHKI

SEQ ID NO: 671 - protein, 6 aa, Artificial Sequence
  Synthetic
MSHKHD

SEQ ID NO: 672 - protein, 6 aa, Artificial Sequence
  Synthetic
GGGGGG

SEQ ID NO: 673 - protein, 6 aa, Artificial Sequence
  Synthetic
GAGAGA

SEQ ID NO: 674 - protein, 12 aa, Artificial Sequence
  Synthetic
FITCEHGMGHNT

SEQ ID NO: 675 - protein, 12 aa, Artificial Sequence
  Synthetic
HXHXHXHXHXHX

SEQ ID NO: 676 - protein, 15 aa, Artificial Sequence
  Synthetic
HXHXXHXHXXHXHXX

SEQ ID NO: 677 - DNA, 69 bp, Artificial Sequence
  Synthetic
aggttccatgggacactcgcattcacacagccactctcacagccattccggagtaggagattatgaaag

SEQ ID NO: 678 - DNA, 72 bp, Artificial Sequence
  Synthetic
aggttccatgggacactcgcattcaagtcacagccactcttcacacagccatggagtaggagattatgaaag

SEQ ID NO: 679 - DNA, 87 bp, Artificial Sequence
  Synthetic
aggttccatgggacactcgcattcaagtcacagccactcttcgcacagccattccagtcacagccacggagtaggagattatgaaag

SEQ ID NO: 680 - DNA, 66 bp, Artificial Sequence
  Synthetic
aggttccatgggacacaaacataagcacaagcacaaacacaagcacggagtaggagattatgaaag

SEQ ID NO: 681 - DNA, 72 bp, Artificial Sequence
  Synthetic
aggttccatgggacacaaacataagaagcacaagcacaagaaacacaagcatggagtaggagattatgaaag

SEQ ID NO: 682 - DNA, 72 bp, Artificial Sequence
  Synthetic
aggttccatgggacactcacattcaagccactatcataagaaacataagcacggagtaggagattatgaaag

SEQ ID NO: 683 - DNA, 72 bp, Artificial Sequence
  Synthetic
aggttccatgggacactatcataagaaacataagcactcgagtcatagccacggagtaggagattatgaaag

SEQ ID NO: 684 - DNA, 87 bp, Artificial Sequence
  Synthetic
aggttccatgggacactcacataagagccactatcataagaaacataagcactacagtcatagccacggagtaggagattatgaaag

SEQ ID NO: 685 - DNA, 72 bp, Artificial Sequence
  Synthetic
aggttccatgggacactcacataagagccactatcattcctcgcataagcacggagtaggagattatgaaag

SEQ ID NO: 686 - DNA, 72 bp, Artificial Sequence
  Synthetic
aggttccatgggacactcacataagagccactatcataagtcgcattctcacggagtaggagattatgaaag

SEQ ID NO: 687 - protein, 5 aa, Artificial Sequence
  Synthetic
GHGHG